(19) United States
(12) Patent Application Publication
(10) Pub. No.: US 2026/0294915 A1
(43) Pub. Date: Oct. 1, 2026

(54) COMBINATION THERAPY COMPRISING A MAT2A INHIBITOR AND A TOPOISOMERASE INHIBITOR, A SPLICING INHIBITOR SULFONAMIDE, OR A KIF18 INHIBITOR

(71) Applicants: IDEAYA BIOSCIENCES, INC., South San Francisco, CA (US); GLAXOSMITHKLINE INTELLECTUAL PROPERTY (NO.4) LIMITED, Brentford, Middlesex (GB)

(72) Inventors: Marcus M. FISCHER, South San Francisco, CA (US); Claire L. NEILAN, South San Francisco, CA (US); Kimberline Yang GERRICK, South San Francisco, CA (US); Anthony MAZUREK, Collegeville, PA (US); Jenny Leigh LARAIO, Collegeville, PA (US)

(21) Appl. No.: 19/474,291

(22) PCT Filed: Apr. 12, 2024
(86) PCT No.: PCT/US2024/024418

§ 371 Date: Oct. 10, 2025

Related U.S. Application Data
(60) Provisional application No. 63/495,911, filed on Apr. 13, 2023.
(60) Provisional application No. 63/598,799, filed on Nov. 14, 2023.

(51) Int. Cl. A61K 31/519; A61K 31/4745; A61P 35/00

(52) U.S. Cl. CPC A61K 31/519; A61K 31/4745; A61P 35/00

(57) ABSTRACT

Provided herein is a combination of a methionine adenosyltransferase II alpha (MAT2A) inhibitor and a topoisomerase inhibitor, a splicing inhibitor sulfonamide, or a KIF18 inhibitor. Also provided are methods of using such combinations to treat diseases or disorders, for example, cancer.

### CROSS-REFERENCES TO RELATED APPLICATIONS

This application claims the benefit to U.S. Provisional Application Nos. 63/495,911 filed Apr. 13, 2023 and 63/598,799 filed Nov. 14, 2023, each of which is incorporated herein in its entirety and for all purposes.

### BACKGROUND

Cancer is a leading cause of death throughout the world. A limitation of prevailing therapeutic approaches, e.g., chemotherapy and immunotherapy, is that their cytotoxic effects are not restricted to cancer cells and adverse side effects can occur within normal tissues.

Methionine adenosyltransferase 2A (MAT2A) is an enzyme that utilizes methionine (Met) and adenosine triphosphate (ATP) to generate s-adenosyl methionine (SAM). SAM is a primary methyl donor in cells used to methylate several substrates including DNA, RNA, and proteins. One methylase that utilizes SAM as a methyl donor is protein arginine N-methyltransferase 5 (PRMT5). While SAM is required for PRMT5 activity, PRMT5 is competitively inhibited by 5′methylthioadenosine (MTA). Since MTA is part of the methionine salvage pathway, cellular MTA levels stay low in a process initiated by methylthioadenosine phosphorylase (MTAP).

MTAP is located at a locus on chromosome 9 that is often deleted in the cells of patients with cancers from several tissues of origin including central nervous system, pancreas, esophageal, bladder and lung (eBioPortal database). Loss of MTAP results in the accumulation of MTA making MTAP-deleted cells more dependent on SAM production, and thus MAT2A activity, compared to cells that express MTAP. In an shRNA cell-line screen across approximately 400 cancer cell lines, MAT2A knockdown resulted in the loss of viability in a larger percentage of MTAP-deleted cells compare to MTAP WT cells (see McDonald et. al. 2017 Cell 170, 577-592). Furthermore, inducible knockdown of MAT2A protein decreased tumor growth in vivo (see Marjon et. al., 2016 Cell Reports 15(3), 574-587). These results indicate that MAT2A inhibitors may provide a novel therapy for cancer patients including those with MTAP-deleted tumors.

DNA-topoisomerases are enzymes that are present in the nuclei of cells where they catalyze the breaking and rejoining of DNA strands, which control the topological state of DNA. Recent studies also suggest that topoisomerases are also involved in regulating template supercoiling during RNA transcription. There are two major classes of mammalian topoisomerase. DNA-topoisomerase-I catalyzes changes in the topological state of duplex DNA by performing transient single-strand breakage-union cycles. In contrast, mammalian topoisomerase II alters the topology of DNA by causing a transient enzyme bridged double-strand break, followed by strand passing and resealing. The antitumor activity associated with agents that are topoisomerase poisons is associated with their ability to stabilize the enzyme-DNA cleavable complex. This drug-induced stabilization of the enzyme-DNA cleavable complex effectively converts the enzyme into a cellular poison.

Indisulam is an aryl sulfonamide drug with selective anti-cancer activity. Its mechanism of action and the basis for its selectivity are unknown. Many cancer cell lines derived from hematopoietic and lymphoid lineages are sensitive to indisulam and their sensitivity correlates with DCAF15 expression levels. Two other clinically tested sulfonamides, tasisulam and CQS, share the same mechanism of action as indisulam. This class of drugs is generally referred to as SPLAMs (splicing inhibitor sulfonamides).

Kinesins are molecular motors that play important roles in cell division and intracellular vesicle and organelle transport. Mitotic kinesin plays roles in several aspects of spindle assembly, chromosome segregation, centrosome separation and dynamics (reviewed in O. Rath and F. Kozielski, Nature Review Cancer, 12:527-39, 2012). Human kinesins are categorized into 14 subfamilies based on sequence homology within the “motor domain.” This domain's ATPase activity drives unidirectional movement along microtubules (MTs). The non-motor domain of these proteins is responsible for cargo attachment. A “cargo” can include any one of a variety of different membranous organelles, signal transduction scaffolding systems, and chromosomes. Kinesins use the energy of ATP hydrolysis to move cargo along polarized microtubules. Thus, kinesins are often called “plus-end” or “minus-end” directed motors.

KIF18A gene belongs to Kinesin-8 subfamily and is a plus-end-directed motor. KIF18A is believed to influence dynamics at the plus end of kinetochore microtubules to control correct chromosome positioning and spindle tension. Depletion of human KIF18A leads to longer spindles, increased chromosome oscillation at metaphase, and activation of the mitotic spindle assembly checkpoint in HeLa cervical cancer cells (M. I. Mayr, et al., Current Biology 17, 488-98, 2007). KIF18A is overexpressed in various types of cancers, including but not limited to colon, breast, lung, pancreas, prostate, bladder, head, neck, cervix, and ovarian cancers. Further, genetic deletion or knockdown, or inhibition of KIF18A effects mitotic spindle apparatus in cancer cell lines. Particularly, inhibition of KIF18A has been found to induce mitotic cell arrest, a known vulnerability that can promote cell death in mitosis via apoptosis, mitotic catastrophe, or multipolarity driven lethality or death after mitotic slippage in interphase. Accordingly, there has been a strong interest in finding inhibitors of KIF18A proteins.

Despite many recent advances in cancer therapies, there remains a need for more effective and/or enhanced treatment for those individuals suffering the effects of cancer.

### SUMMARY

Provided herein is a combination product comprising a methionine adenosyltransferase II alpha (MAT2A) inhibitor and a topoisomerase inhibitor, a splicing inhibitor sulfonamide (SPLAM), or a KIF18 inhibitor. The combination product is useful for the treatment of a variety of cancers, including solid tumors. The combination product is also useful for the treatment of a variety of diseases or disorders treatable by inhibiting MAT2A. The combination product is also useful for treating MTAP-deficient tumors.

In an embodiment, provided herein is a combination of a MAT2A inhibitor and a topoisomerase inhibitor, a splicing inhibitor sulfonamide (SPLAM), or a KIF18 inhibitor.

In an embodiment, provided herein is a pharmaceutical composition comprising a therapeutically effective amount of a methionine adenosyltransferase II alpha (MAT2A) inhibitor and a second pharmaceutical composition comprising a therapeutically effective amount of a topoisomerase inhibitor, a splicing inhibitor sulfonamide (SPLAM), or a KIF18 inhibitor. In some embodiments, the second pharmaceutical composition comprises a topoisomerase inhibitor. In some embodiments, the second pharmaceutical composition comprises a SPLAM. In some embodiments, the second pharmaceutical composition comprises a KIF18 inhibitor.

In an embodiment, provided herein are methods of treating cancer in a subject in need thereof, the methods comprising administering to the subject a combination comprising a MAT2A inhibitor and a topoisomerase inhibitor, a splicing inhibitor sulfonamide (SPLAM), or a KIF18 inhibitor, thereby treating the cancer in the subject.

In an embodiment, provided herein are methods of treating cancer in a subject in need thereof, the methods comprising administering to the subject a combination comprising a MAT2A inhibitor and a topoisomerase inhibitor, a splicing inhibitor sulfonamide (SPLAM), or a KIF18 inhibitor, together with at least a pharmaceutically acceptable carrier, thereby treating the cancer in the subject.

In still another embodiment, the cancer is characterized by a reduction or absence of methylthioadenosine phosphorylase (MTAP) gene expression, an absence of MTAP gene, a reduced function of MTAP protein, a reduced level or absence of MTAP protein, a MTA accumulation, or a combination thereof.

In an embodiment, provided herein are methods of treating cancer in a subject in need thereof, the methods comprising administering to the subject a therapeutically effective amount of a pharmaceutical composition comprising a MAT2A inhibitor and a therapeutically effective amount of a pharmaceutical composition comprising a topoisomerase inhibitor, a splicing inhibitor sulfonamide (SPLAM), or a KIF18 inhibitor, thereby treating the cancer in the subject.

In an embodiment, provided herein are methods of treating a disease or disorder treatable by inhibiting MAT2A in a subject in need thereof, the methods comprising administering to the subject a combination comprising a methionine adenosyltransferase II alpha (MAT2A) inhibitor and a topoisomerase inhibitor, a splicing inhibitor sulfonamide (SPLAM), or a KIF18 inhibitor, thereby treating the disease or disorder in the subject. In an embodiment, the disease or disorder is cancer.

In an embodiment, provided herein are methods of treating a disease or disorder treatable by inhibiting MAT2A in a subject in need thereof, the methods comprising administering to the subject a combination comprising a MAT2A inhibitor and a topoisomerase inhibitor, a splicing inhibitor sulfonamide (SPLAM), or a KIF18 inhibitor, together with at least a pharmaceutically acceptable carrier, thereby treating the disease or disorder in the subject. In an embodiment, the disease or disorder is cancer.

In an embodiment, the MAT2A inhibitor is a compound of Formula (I):

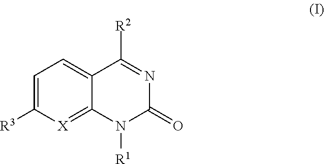

or a pharmaceutically acceptable salt thereof, wherein the variables of Formula (I) are defined below.

In another embodiment, the MAT2A inhibitor is Compound A having the following structural formula:

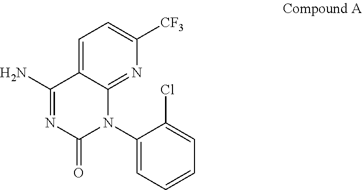 [SMILES: Nc1nc(=O)n(-c2ccccc2Cl)c2nc(C(F)(F)F)ccc12]

or a pharmaceutically acceptable salt thereof.

In another embodiment, the MAT2A inhibitor is Compound A1 having the following structural formula:

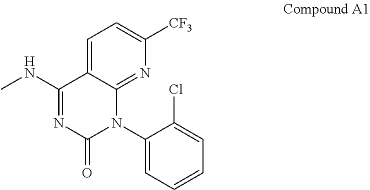 [SMILES: CNc1nc(=O)n(-c2ccccc2Cl)c2nc(C(F)(F)F)ccc12]

or a pharmaceutically acceptable salt thereof.

MAT2A inhibitors for use in the combination therapy described herein are described in WO 2020/123395 (PCT/US19/65260). The generic and specific compounds described in the patent application are incorporated herein by reference and can be used to treat cancer as described herein.

In an embodiment, the KIF18 inhibitor is Compound B:

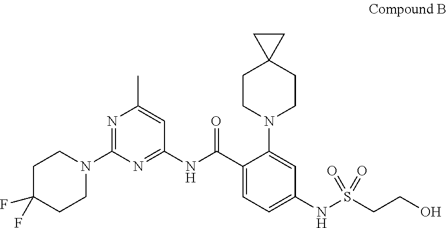 [SMILES: Cc1cc(NC(=O)c2ccc(NS(=O)(=O)CCO)cc2N2CCC3(CC2)CC3)nc(N2CCC(F)(F)CC2)n1]

or a pharmaceutically acceptable salt thereof. The chemical name for Compound B is N-(2-(4,4-difluoropiperidin-1-yl)-6-methylpyrimidin-4-yl)-4-((2-hydroxyethyl)sulfonamido)-2-(6-azaspiro[2.5]octan-6-yl)benzamide. Compound B is also referred to as “sovilnesib” or “AMG650.” Compound B also includes pharmaceutically acceptable salts thereof.

In another embodiment, the SPLAM is indisulam:

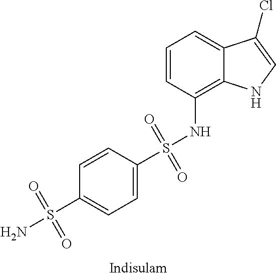 [SMILES: NS(=O)(=O)c1ccc(S(=O)(=O)Nc2cccc3c(Cl)c[nH]c23)cc1]

or a pharmaceutically acceptable salt thereof. The chemical name for indisulam is N-(3-chloro-1H-indol-7-yl)benzene-1,4-disulfonamide. Indisulam also includes.
pharmaceutically acceptable salts thereof.

In yet another embodiment, the SPLAM is E7820:

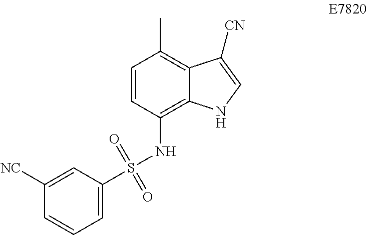 [SMILES: Cc1ccc(NS(=O)(=O)c2cccc(C#N)c2)c2[nH]cc(C#N)c12]

or a pharmaceutically acceptable salt thereof. The chemical name for E7820 is 3-cyano-N-(3-cyano-4-methyl-1H-indol-7-yl)benzenesulfonamide. E7820 also includes pharmaceutically acceptable salts thereof.

In still another embodiment, the SPLAM is chloroquinoxaline sulfonamide:

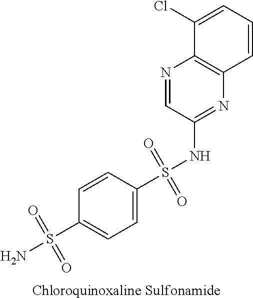 [SMILES: NS(=O)(=O)c1ccc(S(=O)(=O)Nc2cnc3c(Cl)cccc3n2)cc1 ; "Chloroquinoxaline Sulfonamide"]

or a pharmaceutically acceptable salt thereof. The chemical name for chloroquinoxaline sulfonamide is 4-amino-N-(5-chloroquinoxalin-2-yl)benzenesulfonamide, also referred to as “CQS.” Chloroquinoxaline sulfonamide also includes pharmaceutically acceptable salts thereof.

In another embodiment, the SPLAM is tasisulam:

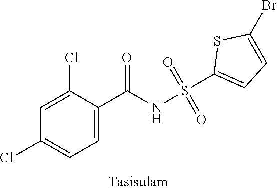 [SMILES: O=C(NS(=O)(=O)c1ccc(Br)s1)c1ccc(Cl)cc1Cl]

or a pharmaceutically acceptable salt thereof. The chemical name for tasisulam is N-((5-bromothiophen-2-yl)sulfonyl)-2,4-dichlorobenzamide. Tasisulam also includes pharmaceutically acceptable salts thereof.

In an embodiment, the topoisomerase inhibitor is a type I topoisomerase inhibitor.

In another embodiment, the type I topoisomerase inhibitor is 10-hydroxycamptothecin:

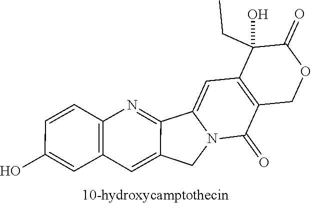 [SMILES: CC[C@@]1(O)C(=O)OCc2c1cc1n(c2=O)Cc2cc3cc(O)ccc3nc2-1]

or a pharmaceutically acceptable salt thereof. The chemical name for 10-hydroxycamptothecin is (S)-4-ethyl-4,9-dihydroxy-1,12-dihydro-14H-pyrano[3′,4′:6,7]indolizino[1,2-b]quinoline-3,14(4H)-dione. 10-hydroxycamptothecin also includes pharmaceutically acceptable salts thereof.

In yet another embodiment, the type I topoisomerase inhibitor is irinotecan:

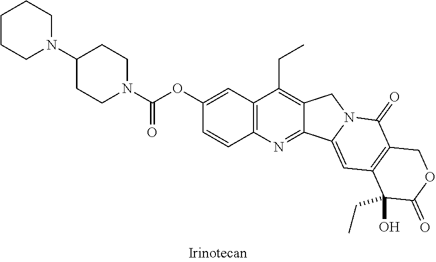 [SMILES: CCc1c2c(nc3ccc(OC(=O)N4CCC(N5CCCCC5)CC4)cc13)-c1cc3c(c(=O)n1C2)COC(=O)[C@]3(O)CC]

or a pharmaceutically acceptable salt thereof. The chemical name for irinotecan is (S)-4,11-diethyl-4-hydroxy-3,14-dioxo-3,4,12,14-tetrahydro-1H-pyrano[3′,4′:6,7]indolizino[1,2-b]quinolin-9-yl [1,4′-bipiperidine]-1′-carboxylate. Irinotecan also includes pharmaceutically acceptable salts thereof.

In still another embodiment, the type I topoisomerase inhibitor is topotecan:

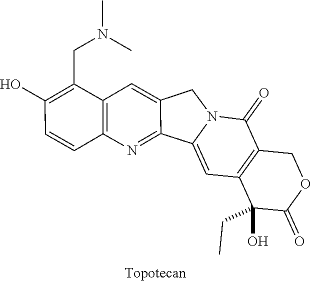 [SMILES: CC[C@@]1(O)C(=O)OCc2c1cc1n(c2=O)Cc2cc3c(CN(C)C)c(O)ccc3nc2-1]

or a pharmaceutically acceptable salt thereof. The chemical name for topotecan is (S)-10-((dimethylamino)methyl)-4-ethyl-4,9-dihydroxy-1,12-dihydro-14H-pyrano[3′,4′:6,7]indolizino[1,2-b]quinoline-3,14(4H)-dione. Topotecan also includes pharmaceutically acceptable salts thereof.

In an embodiment, the topoisomerase inhibitor is a type II topoisomerase inhibitor.

In another embodiment, the type II topoisomerase inhibitor is daunorubicin:

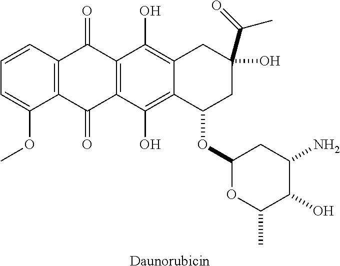

or a pharmaceutically acceptable salt thereof. The chemical name for daunorubicin is (8S,10S)-8-acetyl-10-(((2R,4S,5S,6S)-4-amino-5-hydroxy-6-methyltetrahydro-2H-pyran-2-yl)oxy)-6,8,11-trihydroxy-1-methoxy-7,8,9,10-tetrahydrotetracene-5,12-dione. Daunorubicin also includes pharmaceutically acceptable salts thereof.

In yet another embodiment, the type II topoisomerase inhibitor is doxorubicin:

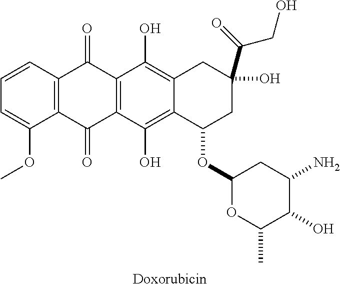

or a pharmaceutically acceptable salt thereof. The chemical name for doxorubicin is (8S,10S)-10-(((2R,4S,5S,6S)-4-amino-5-hydroxy-6-methyltetrahydro-2H-pyran-2-yl)oxy)-6,8,11-trihydroxy-8-(2-hydroxyacetyl)-1-methoxy-7,8,9,10-tetrahydrotetracene-5,12-dione. Doxorubicin also includes pharmaceutically acceptable salts thereof.

In still another embodiment, the type II topoisomerase inhibitor is etoposide:

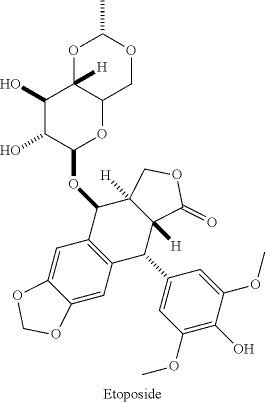

or a pharmaceutically acceptable salt thereof. The chemical name for etoposide is (5R,5aR,8aR,9S)-9-(((2R,6R,7R,8R,8aS)-7,8-dihydroxy-2-methylhexahydropyrano[3,2-d][1,3]dioxin-6-yl)oxy)-5-(4-hydroxy-3,5-dimethoxyphenyl)-5,8,8a,9-tetrahydrofuro[3′,4′:6,7]naphtho[2,3-d][1,3]dioxol-6(5aH)-one. Etoposide also includes pharmaceutically acceptable salts thereof.

### BRIEF DESCRIPTION OF THE DRAWINGS

FIG. 1A-1 to FIG. 1A-5 and FIG. 1B-1 to FIG. 1B-2 show growth inhibition of Compound B (sovilnesib) as a single agentin five MTAP-deficient cell lines (FIG. 1A-1 to FIG. 1A-5) and two wild type cell lines (FIGS. 1B-1 and 1B-2).

FIG. 2A-1 to FIG. 2G show growth inhibition of Compound A and 10-Hydroxycamptothecin in thirteen (13) MTAP-deficient cell lines.

FIG. 3A to FIG. 3G-2 show Loewe Synergy of Compound A and 10-Hydroxycamptothecin in thirteen (13) MTAP-deficient cell lines.

FIG. 4A-1 to FIG. 4G show growth inhibition of Compound A and Irinotecan in thirteen (13) MTAP-deficient cell lines.

FIG. 5A to FIG. 5G-2 show Loewe Synergy of Compound A and Irinotecan in thirteen (13) MTAP-deficient cell lines.

FIG. 6A-1 to FIG. 6G show growth inhibition of Compound A and Topotecan in thirteen (13) MTAP-deficient cell lines.

FIG. 7A to FIG. 7G-2 show Loewe Synergy of Compound A and Topotecan in thirteen (13) MTAP-deficient cell lines.

FIG. 8A-1 to FIG. 8G show growth inhibition of Compound A and Daunorubicin in thirteen (13) MTAP-deficient cell lines.

FIG. 9A to FIG. 9G-2 show Loewe Synergy of Compound A and Daunorubicin in thirteen (13) MTAP-deficient cell lines.

FIG. 10A-1 to FIG. 10G show growth inhibition of Compound A and Doxorubicin in thirteen (13) MTAP-deficient cell lines.

FIG. 11A to FIG. 11G-2 show Loewe Synergy of Compound A and Doxorubicin in thirteen (13) MTAP-deficient cell lines.

FIG. 12A-1 to FIG. 12G show growth inhibition of Compound A and Etoposide in thirteen (13) MTAP-deficient cell lines.

FIG. 13A to FIG. 13G-2 show Loewe Synergy of Compound A and Etoposide in thirteen (13) MTAP-deficient cell lines.

FIG. 14 shows anti-tumor activity of Compound A and irinotecan, alone or in combination, in RT-112/84 bladder CDX model.

FIG. 15 shows change in individual tumor volumes following administration of irinotecan alone or a combination of Compound A and irinotecan in the RT-112/84 CDX model.

FIG. 16 shows Anti-tumor activity of Compound A and irinotecan in Gastric CDX Model MKN45

FIG. 17 shows Anti-tumor activity of Compound A and irinotecan in Gastric CDX Model LMSU

### DETAILED DESCRIPTION

Provided herein is a combination therapy comprising a methionine adenosyltransferase II alpha (MAT2A) inhibitor, or a pharmaceutically acceptable salt thereof, and a topoisomerase inhibitor, a splicing inhibitor sulfonamide (SPLAM), or a KIF18 inhibitor, or pharmaceutically acceptable salts thereof. The combination therapy is useful for the treatment of a variety of cancers. In another aspect, the combination therapy is useful for the treatment of any one of MAT2A-associated diseases. In another aspect, the combination therapy is useful for the treatment of a disease or disorder treatable by inhibiting MAT2A.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±10%, and ±0.1% from the specified value, as such variations are appropriate to perform the disclosed methods.

As used in the specification and in the claims, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. In addition, the phrase “about ‘x’ to ‘y’ includes “about ‘x’ to about ‘y’”.

The terms “combination,” “therapeutic combination,” “pharmaceutical combination,” or “combination product” as used herein refer to either a fixed combination in one dosage unit form, or non-fixed combination in separate dosage forms, or a kit of parts for the combined administration where two or more therapeutic agents may be administered independently, at the same time or separately within time intervals.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (e.g., capsules and/or intravenous formulations) for each active ingredient. In addition, such administration also encompasses use of each type of therapeutic agent in a sequential or separate manner, either at approximately the same time or at different times. Regardless of whether the active ingredients are administered as a single formulation or in separate formulations, the drugs are administered to the same patient as part of the same course of therapy. In any case, the treatment regimen will provide beneficial, e.g., synergistic, effects in treating the conditions or disorders described herein.

As used herein, the term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease.

As used herein, the term “prevent” or “prevention” means no disorder or disease development if none had occurred, or no further disorder or disease development if there had already been development of the disorder or disease. Also considered is the ability of the combination therapy provided herein to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system.

An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein a parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts described herein include the conventional non-toxic salts of the parent compound formed, for example, from non-toxic inorganic or organic acids. The pharmaceutically acceptable salts discussed herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound with a pharmaceutically acceptable carrier. The pharmaceutical composition facilitates administration of the composition to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound disclosed herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of a compound disclosed herein, and are physiologically acceptable to the patient. Supplementary active compounds may also be incorporated into the compositions. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

The term “single formulation” as used herein refers to a single carrier or vehicle formulated to deliver therapeutically effective amounts of both therapeutic agents to a patient. The single vehicle is designed to deliver a therapeutically effective amount of each of the agents, along with any pharmaceutically acceptable carriers or excipients. In some embodiments, the vehicle is a tablet, capsule, pill, or a patch. In other embodiments, the vehicle is a solution or a suspension.

As used herein “methionine adenosyltransferase II alpha inhibitor” or “MAT2A inhibitor” means an agent that modulates the activity of MAT2A or inhibits the production of S-adenosylmethionine (SAM) by methionine adenosyltransferase 2A (MAT2A).

The combination of agents described herein may display a synergistic effect. The term “synergistic effect” or “synergy” as used herein, refers to action of two agents such as, for example, a MAT2A inhibitor and a topoisomerase inhibitor, a splicing inhibitor sulfonamide (SPLAM), or a KIF 18 inhibitor, producing an effect, for example, slowing the symptomatic progression of cancer or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

In an embodiment, provided herein is a combination therapy comprising a therapeutically effective amount of a MAT2A inhibitor; and a topoisomerase inhibitor, a splicing inhibitor sulfonamide (SPLAM), or a KIF18 inhibitor. A “therapeutically effective amount” of a combination of agents (i.e., a MAT2A inhibitor in combination with a topoisomerase inhibitor, a splicing inhibitor sulfonamide (SPLAM), or a KIF18 inhibitor) is an amount sufficient to provide an observable improvement over the baseline clinically observable signs and symptoms of the disorders treated with the combination.

“Alkyl” means a linear saturated monovalent hydrocarbon radical of one to six carbon (i.e. C₁₋₆ means one to six carbons) atoms or a branched saturated monovalent hydrocarbon radical of three to six carbon atoms (i.e. C₃₋₆ means three to six carbons). Alkyl can include any number of carbons, such as C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. Example of alkyl groups include methyl, ethyl, propyl, 2-propyl, butyl, pentyl, and the like. It will be recognized by a person skilled in the art that the term “alkyl” may include “alkylene” groups.

“Amino” means a —NH₂.

“Cycloalkyl” means a monocyclic monovalent hydrocarbon radical of three to six carbon atoms (e.g., C₃₋₆ cycloalkyl) which may be saturated or contains one double bond. Cycloalkyl can include any number of carbons, such as C₃₋₆, C₄₋₆, and C₅₋₆. Partially unsaturated cycloalkyl groups have one or more double in the ring, but cycloalkyl groups are not aromatic. Saturated monocyclic cycloalkyl rings include, for example, cyclopropyl, cyclobutyl, cyclopentyl, and cyclohexyl.

“Halo” means fluoro, chloro, bromo, or iodo, preferably fluoro or chloro.

“Haloalkyl” means alkyl radical as defined above, which is substituted with one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to as fluoroalkyl. As for alkyl group, haloalkyl groups can have any suitable number of carbon atoms, such as C₁₋₆.

### Combination Product

Provided herein is a combination product comprising a MAT2A inhibitor, or a pharmaceutically acceptable salt thereof; and a topoisomerase inhibitor, a splicing inhibitor sulfonamide (SPLAM), or a KIF18 inhibitor, or pharmaceutically acceptable salts thereof. This combination product is also referred to herein as a combination therapy. The combination product is useful for the treatment of a variety of cancers, including solid tumors. In another aspect, the combination product is useful for the treatment of any one of MAT2A-associated diseases. In another aspect, the combination product is useful for the treatment of a disease or disorder treatable by inhibiting MAT2A. In another aspect, the combination product is useful for treating MTAP-deficient tumors.

In an embodiment, provided herein is a combination of a MAT2A inhibitor and a topoisomerase inhibitor, a splicing inhibitor sulfonamide (SPLAM), or a KIF18 inhibitor. In some embodiments, provided herein is a combination of a MAT2A inhibitor and a topoisomerase inhibitor. In some embodiments, provided herein is a combination of a MAT2A inhibitor and a SPLAM. In some embodiments, provided herein is a combination of a MAT2A inhibitor and a KIF18 inhibitor.

The disclosure provides MAT2A inhibitors. In an embodiment, the MAT2A inhibitor is a compound or a pharmaceutically acceptable salt thereof of Formula (I):

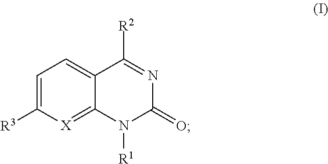

wherein
   X is CH or N;
   R³ is halo, C₁₋₆ haloalkyl or C₃₋₆ cycloalkyl;
   R² is —NR⁴R⁵;
   R⁴ is hydrogen or C₁₋₆ alkyl;
   R⁵ is hydrogen, C₁₋₆ alkyl or C₃₋₆ cycloalkyl; and
   R¹ is phenyl, wherein phenyl is substituted with 0-2 halo.

In an embodiment, X in Formula (I) and subembodiments thereof is CH. In an embodiment, X in Formula (I) and subembodiments thereof is N.

In still another embodiment, R³ in formula (I) and subembodiments thereof is halo or C₁₋₆ haloalkyl. In an embodiment, R³ in formula (I) and subembodiments thereof is halo. In an embodiment, R³ in formula (I) and subembodiments thereof is C₁₋₆ haloalkyl. In an embodiment, R³ in formula (I) and subembodiments thereof is C₃₋₆ cycloalkyl. In an embodiment, R³ in formula (I) and subembodiments thereof is chloro, fluoro, bromo, —CH₂Cl, —CF₃, —CHF₂, —CH₂CF₃, —CF₂CF₃, or —CF(CH₃)₂. In an embodiment, R³ in formula (I) and subembodiments thereof is chloro or —CF₃. In an embodiment, R³ in formula (I) and subembodiments thereof is chloro. In an embodiment, R³ in formula (I) and subembodiments thereof is —CF₃.

In still another embodiment, R⁴ in formula (I) and subembodiments thereof is H. In an embodiment, R⁴ in formula (I) and subembodiments thereof is C₁₋₃alkyl. In an embodiment, R⁴ in formula (I) and subembodiments thereof is methyl, ethyl, propyl, or isopropyl.

In still another embodiment, R⁵ in formula (I) and subembodiments thereof is H. In an embodiment, R⁵ in formula (I) and subembodiments thereof is C₁₋₃alkyl. In an embodiment, R⁵ in formula (I) and subembodiments thereof is methyl, ethyl, propyl, or isopropyl. In an embodiment, R⁵ in formula (I) and subembodiments thereof is C₃₋₆ cycloalkyl.

In some embodiments of formula (I) and subembodiments thereof, R⁴ and R⁵ are each H. In some embodiments of formula (I) and subembodiments thereof, one of R⁴ and R⁵ is H, and the other one is C₁₋₃alkyl. In some embodiments of formula (I) and subembodiments thereof, one of R⁴ and R⁵ is H, and the other one is methyl, ethyl, propyl, or isopropyl. In some embodiments of formula (I) and subembodiments thereof, R⁴ and R⁵ are independently C₁₋₃alkyl.

In still another embodiment, R² in formula (I) and subembodiments thereof is —NH₂, —NHC₁₋₃alkyl, or —N(C₁₋₃alkyl)₂. In an embodiment, R² in formula (I) and subembodiments thereof is NH₂, —NHMe, or —N(Me)₂. In an embodiment, R² in formula (I) and subembodiments thereof is NH₂. In an embodiment, R² in formula (I) and subembodiments thereof is —NHMe.

In an embodiment, R¹ in formula (I) and subembodiments thereof is phenyl, which is unsubstituted or substituted with 1-2 halo. In still another embodiment, R¹ in formula (I) and subembodiments thereof is unsubstituted phenyl. In an embodiment, R¹ in formula (I) and subembodiments thereof is phenyl substituted with 1 halo. In an embodiment, R¹ in formula (I) and subembodiments thereof is phenyl substituted 1 halo selected fluoro and chloro. In an embodiment, R¹ in formula (I) and subembodiments thereof is phenyl substituted chloro. In an embodiment, R¹ in formula (I) and subembodiments thereof is phenyl substituted 2 halo.

In yet another embodiment, the MAT2A inhibitor is selected from the group consisting of a compound from Table 1, or a pharmaceutically acceptable salt thereof.

TABLE 1

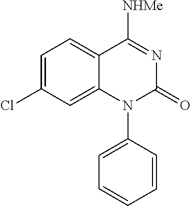 [SMILES: CNc1nc(=O)n(-c2ccccc2)c2cc(Cl)ccc12]

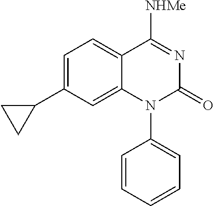 [SMILES: CNc1nc(=O)n(-c2ccccc2)c2cc(C3CC3)ccc12]

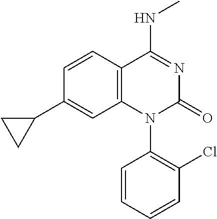 [SMILES: CNc1nc(=O)n(-c2ccccc2Cl)c2cc(C3CC3)ccc12]

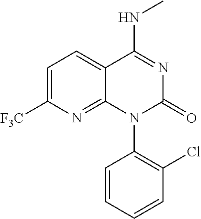 [SMILES: CNc1nc(=O)n(-c2ccccc2Cl)c2nc(C(F)(F)F)ccc12]

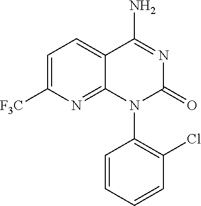 [SMILES: Nc1nc(=O)n(-c2ccccc2Cl)c2nc(C(F)(F)F)ccc12]

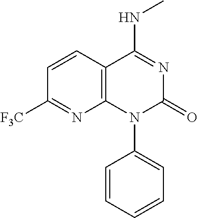 [SMILES: CNc1nc(=O)n(-c2ccccc2)c2nc(C(F)(F)F)ccc12]

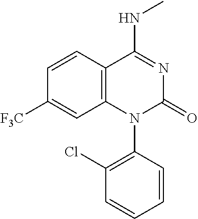 [SMILES: CNc1nc(=O)n(-c2ccccc2Cl)c2cc(C(F)(F)F)ccc12]

In another embodiment, the MAT2A inhibitor is Compound A:

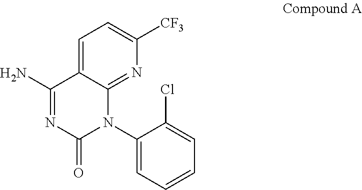 [SMILES: Nc1nc(=O)n(-c2ccccc2Cl)c2nc(C(F)(F)F)ccc12]

or a pharmaceutically acceptable salt thereof.

In another embodiment, the MAT2A inhibitor is Compound A1 having the following structural formula:

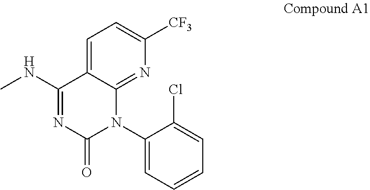 [SMILES: CNc1nc(=O)n(-c2ccccc2Cl)c2nc(C(F)(F)F)ccc12]

or a pharmaceutically acceptable salt thereof.

The preparation and activity of the MAT2A inhibitors provided herein are disclosed in PCT/US2019/065260 (WO 2020/123395), the entire content of which is hereby incorporated by reference in its entirety.

In an embodiment, the combination product comprises a MAT2A inhibitor and a KIF18 inhibitor. In another embodiment, the KIF 18 inhibitor is sovilnesib, or a pharmaceutically acceptable salt thereof. The preparation and activity of sovilnesib are disclosed in PCT/US2019/038169 (WO 2020/132648), the entire content of which is hereby incorporated by reference in its entirety.

In yet another embodiment, the combination product comprises a MAT2A inhibitor and a SPLAM.

In still another embodiment, the SPLAM is indisulam, or a pharmaceutically acceptable salt thereof. The preparation and activity of E7820 are disclosed in PCT/JP1994/001487 (WO 1995/007276) and U.S. Pat. No. 5,767,283, the entire content of which is hereby incorporated by reference in its entirety.

In an embodiment, the SPLAM is E7820, or a pharmaceutically acceptable salt thereof. The preparation and activity of E7820 are disclosed in PCT/JP2000/001071 (WO 2000/050395) and U.S. Pat. No. 6,469,043, the entire content of which is hereby incorporated by reference in its entirety.

In another embodiment, the SPLAM is chloroquinoxaline sulfonamide, or a pharmaceutically acceptable salt thereof. The preparation and activity of chloroquinoxaline sulfonamide are disclosed in PCT/US2000/000191 (WO 2000/040269), the entire content of which is hereby incorporated by reference in its entirety.

In yet another embodiment, the SPLAM is tasisulam, or a pharmaceutically acceptable salt thereof. In still another embodiment, the SPLAM is tasisulam sodium. The preparation and activity of tasisulam are disclosed in PCT/US2002/031568 (WO 2003/035629), the entire content of which is hereby incorporated by reference in its entirety.

In an embodiment, the combination product comprises a MAT2A inhibitor and a topoisomerase inhibitor.

In another embodiment, the topoisomerase inhibitor is a type I topoisomerase inhibitor.

In yet another embodiment, the type I topoisomerase inhibitor is 10-hydroxycamptothecin. The preparation and activity of 10-hydroxycamptothecin are disclosed in PCT/US1990/005986 (WO 1991/005556), the entire content of which is hereby incorporated by reference in its entirety.

In still another embodiment, the type I topoisomerase inhibitor is irinotecan. The preparation and activity of irinotecan are disclosed in PCT/US1992/009864 (WO 1993/09782), the entire content of which is hereby incorporated by reference in its entirety.

In an embodiment, the type I topoisomerase inhibitor is topotecan. The preparation and activity of topotecan are disclosed in PCT/US1992/001029 (WO 1992/014470), the entire content of which is hereby incorporated by reference in its entirety.

In an embodiment, the type I topoisomerase inhibitor is hexylresorcinol, exatecan, deruxtecan, and belotecan, or a pharmaceutically acceptable salt thereof.

In another embodiment, the topoisomerase inhibitor is a type II topoisomerase inhibitor.

In yet another embodiment, the type II topoisomerase inhibitor is daunorubicin. The preparation and activity of daunorubicin are disclosed in U.S. Pat. No. 4,138,480, the entire content of which is hereby incorporated by reference in its entirety.

In still another embodiment, the type II topoisomerase inhibitor is doxorubicin. The preparation and activity of doxorubicin are disclosed in U.S. Pat. No. 4,138,480, the entire content of which is hereby incorporated by reference in its entirety.

In an embodiment, the type II topoisomerase inhibitor is etoposide. The preparation and activity of etoposide are disclosed in PCT/GB1983/000257 (WO 1984/001506), the entire content of which is hereby incorporated by reference in its entirety.

### Methods of Treatment

In an embodiment, provided herein is a method of treating cancer in a subject in need thereof comprising administering to the subject a MAT2A inhibitor and a KIF18 inhibitor, wherein the MAT2A inhibitor is a compound of Formula (I) or a pharmaceutically acceptable salt thereof:

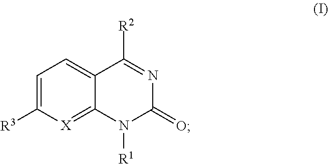

wherein
X is N or CH;
R³ is C₁₋₆ haloalkyl, halo, or C₃₋₆ cycloalkyl;
R² is —NR⁴R⁵;
R⁴ is hydrogen or C₁₋₆ alkyl;
R⁵ is hydrogen, C₁₋₆ alkyl, or C₃₋₆ cycloalkyl; and
R¹ is phenyl, wherein phenyl is substituted with 0-2 halo.

In another embodiment, provided herein is a method of treating cancer in a subject in need thereof comprising administering to the subject a therapeutically effective amount of a MAT2A inhibitor and a therapeutically effective amount of KIF18 inhibitor, wherein the MAT2A inhibitor is a compound of Formula (I) or a pharmaceutically acceptable salt thereof.

In an embodiment, the KIF18 inhibitor is a KIF18A inhibitor. In another embodiment, the KIF18A inhibitor is sovilnesib, or a pharmaceutically acceptable salt thereof. In yet another embodiment, the MAT2A inhibitor is administered simultaneously or sequentially with the KIF18 inhibitor.

In another embodiment, provided herein is a method of treating cancer in a subject in need thereof comprising administering to the subject a MAT2A inhibitor and a splicing inhibitor sulfonamide (SPLAM), wherein the MAT2A inhibitor is a compound of Formula (I) or a pharmaceutically acceptable salt thereof.

In another embodiment, provided herein is a method of treating cancer in a subject in need thereof comprising administering to the subject a therapeutically effective amount of a MAT2A inhibitor and a therapeutically effective amount of a splicing inhibitor sulfonamide (SPLAM), wherein the MAT2A inhibitor is a compound of Formula (I) or a pharmaceutically acceptable salt thereof.

In yet another embodiment, the SPLAM is indisulam, or a pharmaceutically acceptable salt thereof. In still another embodiment, the SPLAM is selected from the group consisting of E7820, chloroquinoxaline sulfonamide, and tasisulam, or a pharmaceutically acceptable salt thereof. In an embodiment, the MAT2A inhibitor is administered simultaneously or sequentially with the SPLAM.

In another embodiment, provided herein is a method of treating cancer in a subject in need thereof comprising administering to the subject a MAT2A inhibitor and a topoisomerase inhibitor, wherein the MAT2A inhibitor is a compound of Formula (I) or a pharmaceutically acceptable salt thereof.

In another embodiment, provided herein is a method of treating cancer in a subject in need thereof comprising administering to the subject a therapeutically effective amount of a MAT2A inhibitor and a therapeutically effective amount of a topoisomerase inhibitor, wherein the MAT2A inhibitor is a compound of Formula (I) or a pharmaceutically acceptable salt thereof.

In another embodiment, provided herein is a method of treating cancer in a subject in need thereof comprising administering to the subject a MAT2A inhibitor and an antibody drug conjugate (ADC) comprising a topoisomerase inhibitor, wherein the MAT2A inhibitor is a compound of Formula (I) or a pharmaceutically acceptable salt thereof.

In yet another embodiment, the topoisomerase inhibitor is a Type I topoisomerase inhibitor. In still another embodiment, the Type I topoisomerase inhibitor is selected from the group consisting of 10-hydroxycamptothecin, irinotecan, and topotecan, or a pharmaceutically acceptable salt thereof. In still another embodiment, the Type I topoisomerase inhibitor is selected from the group consisting of hexylresorcinol, exatecan, deruxtecan, and belotecan, or a pharmaceutically acceptable salt thereof.

In still another embodiment, the ADC comprising the topoisomerase inhibitor is a Type I topoisomerase inhibitor. In still another embodiment, the ADC comprising the topoisomerase inhibitor is fam-trastuzumab deruxtecan-nxki. In still another embodiment, the ADC comprising the topoisomerase inhibitor is AZD8205. In still another embodiment, the ADC comprising the topoisomerase inhibitor is DS-1062 (also known as datopotamab deruxtecan).

In an embodiment, the topoisomerase inhibitor is a Type II topoisomerase inhibitor. In another embodiment, the Type II topoisomerase inhibitor is selected from the group consisting of daunorubicin, doxorubicin, and etoposide, or a pharmaceutically acceptable salt thereof. In still another embodiment, the Type II topoisomerase inhibitor is Amsacrine (m-AMSA) or a pharmaceutically acceptable salt thereof. In yet another embodiment, the MAT2A inhibitor is administered simultaneously or sequentially with the topoisomerase inhibitor.

With reference to the MAT2A of Formula (I), in an embodiment, X is N. In another embodiment, X is CH.

With reference to the MAT2A of Formula (I), in yet another embodiment, R⁴ is hydrogen and R⁵ is hydrogen or C₁₋₃ alkyl. In still another embodiment, R⁵ is hydrogen or methyl.

With reference to the MAT2A of Formula (I), in an embodiment, R¹ is phenyl substituted with chloro.

With reference to the MAT2A of Formula (I), in another embodiment, R³ is C₁₋₃ haloalkyl or halo. In yet another embodiment, R³ is trifluoromethyl or chloro. In still another embodiment, R³ is cyclopropyl.

In an embodiment, the MAT2A inhibitor is selected from the group consisting of a compound from Table 1, or a pharmaceutically acceptable salt thereof. In another embodiment, the MAT2A inhibitor is Compound A, or a pharmaceutically acceptable salt thereof. In yet another embodiment, the MAT2A inhibitor is Compound A1, or a pharmaceutically acceptable salt thereof.

In an embodiment, provided herein are methods of treating cancer in a subject in need thereof, the methods comprising administering to the subject a combination comprising a MAT2A inhibitor and a topoisomerase inhibitor, a splicing inhibitor sulfonamide (SPLAM), or a KIF18 inhibitor, together with at least a pharmaceutically acceptable carrier, thereby treating the cancer in the subject. In some embodiments, provided herein are methods of treating cancer in a subject in need thereof, the methods comprising administering to the subject a combination comprising a MAT2A inhibitor and a topoisomerase inhibitor, together with at least a pharmaceutically acceptable carrier, thereby treating the cancer in the subject. In some embodiments, provided herein are methods of treating cancer in a subject in need thereof, the methods comprising administering to the subject a combination comprising a MAT2A inhibitor and a splicing inhibitor sulfonamide (SPLAM), together with at least a pharmaceutically acceptable carrier, thereby treating the cancer in the subject. In some embodiments, provided herein are methods of treating cancer in a subject in need thereof, the methods comprising administering to the subject a combination comprising a MAT2A inhibitor and a KIF18 inhibitor, together with at least a pharmaceutically acceptable carrier, thereby treating the cancer in the subject.

In still another embodiment, the cancer is characterized by a reduction or absence of methylthioadenosine phosphorylase (MTAP) gene expression, an absence of MTAP gene, a reduced function of MTAP protein, a reduced level or absence of MTAP protein, a MTA accumulation, or a combination thereof.

In an embodiment, provided herein are methods of treating cancer in a subject in need thereof, the methods comprising administering to the subject a therapeutically effective amount of a pharmaceutical composition comprising a MAT2A inhibitor and a therapeutically effective amount of a pharmaceutical composition comprising a topoisomerase inhibitor, a splicing inhibitor sulfonamide (SPLAM), or a KIF18 inhibitor, thereby treating the cancer in the subject. In some embodiments, provided herein are methods of treating cancer in a subject in need thereof, the methods comprising administering to the subject a therapeutically effective amount of a pharmaceutical composition comprising a MAT2A inhibitor and a therapeutically effective amount of a pharmaceutical composition comprising a topoisomerase inhibitor, thereby treating the cancer in the subject. In some embodiments, provided herein are methods of treating cancer in a subject in need thereof, the methods comprising administering to the subject a therapeutically effective amount of a pharmaceutical composition comprising a MAT2A inhibitor and a therapeutically effective amount of a pharmaceutical composition comprising a splicing inhibitor sulfonamide (SPLAM), thereby treating the cancer in the subject. In some embodiments, provided herein are methods of treating cancer in a subject in need thereof, the methods comprising administering to the subject a therapeutically effective amount of a pharmaceutical composition comprising a MAT2A inhibitor and a therapeutically effective amount of a pharmaceutical composition comprising a KIF18 inhibitor, thereby treating the cancer in the subject.

In an embodiment, use of a combination of a MAT2A inhibitor and a topoisomerase inhibitor, a splicing inhibitor sulfonamide (SPLAM), or a KIF18 inhibitor for the manufacture of a medicament is provided. In one embodiment, the MAT2A inhibitor is Compound A. In one embodiment, the MAT2A inhibitor is Compound A1. In an embodiment, the KIF18 inhibitor is sovilnesib. In another embodiment, the SPLAM is indisulam. In yet another embodiment, the SPLAM is selected from the group consisting of E7820, chloroquinoxaline sulfonamide, and tasisulam, or a pharmaceutically acceptable salt thereof. In another embodiment, the topoisomerase inhibitor is selected from the group consisting of 10-hydroxycamptothecin, irinotecan, and topotecan, or a pharmaceutically acceptable salt thereof. In another embodiment, the topoisomerase inhibitor is selected from the group consisting of daunorubicin, doxorubicin, and etoposide, or a pharmaceutically acceptable salt thereof.

In an embodiment of the methods, the MAT2A inhibitor is a compound or a pharmaceutically acceptable salt thereof of Formula (I):

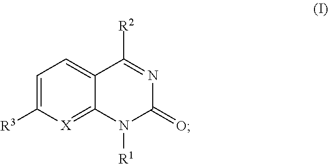

wherein the variables are defined supra.

In another embodiment of the methods, the MAT2A inhibitor is selected from the group consisting of a compound from Table 1, or a pharmaceutically acceptable salt thereof.

In yet another embodiment of the methods, the MAT2A inhibitor is Compound A or a pharmaceutically acceptable salt thereof.

In yet another embodiment of the methods, the MAT2A inhibitor is Compound A1 or a pharmaceutically acceptable salt thereof.

In an embodiment of the methods, the cancer is selected from the group consisting of leukemia, glioma, lung cancer, esophageal cancer, MTAP-deficient pancreatic ductal adenocarcinoma (PDAC), melanoma, pancreatic, non-small cell lung cancer, bladder cancer, astrocytoma, osteosarcoma, head and neck cancer, myxoid chondrosarcoma, ovarian cancer, endometrial cancer, breast cancer, anal cancer, stomach cancer, colon cancer, colorectal cancer, soft tissue sarcoma, non-Hodgkin lymphoma, gastric cancer, esophagogastric cancer, malignant peripheral nerve sheath tumor, mesothelioma, salivary gland tumors, urothelial cancers, gastrointestinal cancer, and sarcomas.

In another embodiment, provided is a product comprising a MAT2A inhibitor and a topoisomerase inhibitor, a splicing inhibitor sulfonamide (SPLAM), or a KIF18 inhibitor as a combined preparation for simultaneous, separate, or sequential use in medicine. In one embodiment, provided is a product comprising a MAT2A inhibitor and a topoisomerase inhibitor as a combined preparation for simultaneous, separate, or sequential use in medicine. In one embodiment, provided is a product comprising a MAT2A inhibitor and a splicing inhibitor sulfonamide (SPLAM) as a combined preparation for simultaneous, separate, or sequential use in medicine. In another embodiment, provided is a product comprising a MAT2A inhibitor and a KIF18 inhibitor as a combined preparation for simultaneous, separate, or sequential use in medicine. In one embodiment, the MAT2A inhibitor is a compound of Formula (I). In one embodiment, the MAT2A inhibitor is a compound in Table 1. In one embodiment, the MAT2A inhibitor is Compound A. In one embodiment, the MAT2A inhibitor is Compound A1. In an embodiment, the KIF18 inhibitor is sovilnesib. In another embodiment, the SPLAM is indisulam. In yet another embodiment, the SPLAM is selected from the group consisting of E7820, chloroquinoxaline sulfonamide, and tasisulam, or a pharmaceutically acceptable salt thereof. In another embodiment, the topoisomerase inhibitor is selected from the group consisting of 10-hydroxycamptothecin, irinotecan, and topotecan, or a pharmaceutically acceptable salt thereof. In another embodiment, the topoisomerase inhibitor is selected from the group consisting of daunorubicin, doxorubicin, and etoposide, or a pharmaceutically acceptable salt thereof.

In yet another embodiment, the cancer is selected from the group consisting of leukemia, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, anal cancer, stomach cancer, colon cancer, colorectal cancer, soft tissue sarcoma, non-Hodgkin lymphoma, gastric cancer, esophagogastric cancer, esophageal cancer, malignant peripheral nerve sheath tumor, and mesothelioma.

In an embodiment, the cancer is mesothelioma. In an embodiment, the cancer is non-small cell lung cancer. In another embodiment, the cancer is nonsquamous non-small cell lung cancer. In one embodiment, the cancer is cancer of the colon or rectum. In an embodiment, the cancer is adenocarcinoma of the colon or rectum. In an embodiment, the cancer is breast cancer. In an embodiment, the cancer is adenocarcinoma of the breast. In an embodiment, the cancer is gastric cancer. In an embodiment, the cancer is gastric adenocarcinoma. In an embodiment, the cancer is pancreatic cancer. In an embodiment, the cancer is pancreatic adenocarcinoma. In an embodiment, the cancer is bladder cancer.

In an embodiment, the cancer is characterized as being MTAP-null.

In an embodiment, the cancer is characterized as being MTAP-deficient.

In still another embodiment, the cancer is a solid tumor. In still another embodiment, the cancer is a MTAP-deleted solid tumor. In still another embodiment, the cancer is a metastatic MTAP-deleted solid tumor.

In still another embodiment, the cancer is metastatic.

In still another embodiment, the cancer is a solid malignant tumor.

In still another embodiment, the cancer is MTAP-deficient lung or MTAP-deficient pancreatic cancer, including MTAP-deficient NSCLC or MTAP-deficient pancreatic ductal adenocarcinoma (PDAC) or MTAP-deficient esophageal cancer.

In another embodiment, the cancer is a tumor having an MTAP gene deletion.

In any one of the embodiments herein, the cancer is a solid tumor or a haematological cancer. In one embodiment, the tumor is characterized by a deficiency in MTAP. In another embodiment, the tumor is characterized by a normal expression of MTAP.

In still another embodiment, the cancer is NSCLC, mesothelioma, squamous carcinoma of the head and neck, salivary gland tumors, urothelial cancers, sarcomas, or ovarian cancer. In still another embodiment, the cancer is NSCLC, esophagogastric and pancreatic cancers. In still another embodiment, the cancer is bladder cancer or gastrointestinal cancer.

In still another embodiment, the cancer is characterized by a reduction or absence of MTAP gene expression, an absence of MTAP gene, a reduced function of MTAP protein, a reduced level or absence of MTAP protein, a MTA accumulation, or a combination thereof.

In still another embodiment, the cancer is characterized by a reduction or absence of MTAP gene expression.

In still another embodiment, the cancer is characterized by a reduced function of MTAP protein.

In still another embodiment, the cancer is characterized by a reduced level or absence of MTAP protein.

In still another embodiment, the cancer is characterized by a MTA accumulation.

In still another embodiment, the cancer is a tumor having PRKC fusions.

In an embodiment, provided herein is a method of inhibiting tumor growth or slowing the rate of tumor growth in a subject having a MTAP-deficient cancer, the method comprising administration of a therapeutically effective amount of a MAT2A inhibitor and a therapeutically effective amount of a topoisomerase inhibitor.

In another embodiment, provided herein is a method of inhibiting tumor growth or slowing the rate of tumor growth in a subject having a MTAP-deficient cancer, the method comprising administration of a therapeutically effective amount of a MAT2A inhibitor and a therapeutically effective amount of a SPLAM.

In still another embodiment, provided herein is a method of inhibiting tumor growth or slowing the rate of tumor growth in a subject having a MTAP-deficient cancer, the method comprising administration of a therapeutically effective amount of a MAT2A inhibitor and a therapeutically effective amount of a KIF18 inhibitor.

Tumor growth is generally measured by a change in a tumor volume taken at a first time point to a second time point. In one embodiment, the tumor growth is measured by a change of from a tumor volume at a first time point to a tumor volume at a second time point. In some embodiments, the tumor volume at the second time point has no increase when compared to the first time point. In some embodiments, the tumor volume decreases from the first time point to the second time point.

In an embodiment, provided herein is a method of treating cancer in a subject in need thereof, the method comprising administering to the subject a therapeutically effective amount of a MAT2A inhibitor and a therapeutically effective amount of a topoisomerase inhibitor, wherein the subject is not previously treated with a MAT2A inhibitor. In some embodiments, the treatment decreases the rate of tumor growth when compared to a treatment with the MAT2A inhibitor alone for a similar time period.

In another embodiment, provided herein is a method of treating cancer in a subject in need thereof, the method comprising administering to the subject a therapeutically effective amount of a MAT2A inhibitor and a therapeutically effective amount of a SPLM, wherein the subject is not previously treated with a MAT2A inhibitor. In some embodiments, the treatment decreases the rate of tumor growth when compared to a treatment with the MAT2A inhibitor alone for a similar time period.

In still another embodiment, provided herein is a method of treating cancer in a subject in need thereof, the method comprising administering to the subject a therapeutically effective amount of a MAT2A inhibitor and a therapeutically effective amount of a KIF8, wherein the subject is not previously treated with a MAT2A inhibitor. In some embodiments, the treatment decreases the rate of tumor growth when compared to a treatment with the MAT2A inhibitor alone for a similar time period.

In an embodiment, the MAT2A inhibitor and the topoisomerase inhibitor, the splicing inhibitor sulfonamide (SPLAM), or the KIF18 inhibitor are in separate dosage forms. In one embodiment, the MAT2A inhibitor and the topoisomerase inhibitor are in separate dosage forms. Ine one embodiment, the MAT2A inhibitor and the splicing inhibitor sulfonamide (SPLAM) are in separate dosage forms. In another embodiment, the MAT2A inhibitor and the KIF18 inhibitor are in separate dosage forms.

In another embodiment, the MAT2A inhibitor and the topoisomerase inhibitor, the splicing inhibitor sulfonamide (SPLAM), or the KIF18 inhibitor are in the same dosage form. In one embodiment, the MAT2A inhibitor and the topoisomerase inhibitor are in the same dosage form. In one embodiment, the MAT2A inhibitor and the splicing inhibitor sulfonamide (SPLAM) are in the same dosage form. In another embodiment, the MAT2A inhibitor and the KIF18 inhibitor are in the same dosage form.

In another embodiment, the treatment comprises administering the MAT2A inhibitor, or a pharmaceutically acceptable salt thereof, and the topoisomerase inhibitor, the splicing inhibitor sulfonamide (SPLAM), or the KIF18 inhibitor, or a pharmaceutically acceptable salt thereof, at substantially the same time. In yet another embodiment, the treatment comprises administering the MAT2A inhibitor, or a pharmaceutically acceptable salt thereof, and the topoisomerase inhibitor, the splicing inhibitor sulfonamide (SPLAM), or the KIF18 inhibitor, or a pharmaceutically acceptable salt thereof, at different times.

In still another embodiment, the MAT2A inhibitor, or a pharmaceutically acceptable salt thereof, is administered to the subject, followed by administration of the topoisomerase inhibitor, the splicing inhibitor sulfonamide (SPLAM), or the KIF18 inhibitor, or a pharmaceutically acceptable salt thereof. In an embodiment, the topoisomerase inhibitor, the splicing inhibitor sulfonamide (SPLAM), or the KIF18 inhibitor, or a pharmaceutically acceptable salt thereof, is administered to the subject, followed by administration of MAT2A inhibitor, or a pharmaceutically acceptable salt thereof.

In some embodiments, the MAT2A inhibitor and topoisomerase inhibitor are administered concomitantly. In some embodiments, the MAT2A inhibitor and the topoisomerase inhibitor are administered sequentially. In some embodiments, the MAT2A inhibitor is administered prior to the administration of the topoisomerase inhibitor. In some embodiments, the MAT2A inhibitor is administered after the administration of the topoisomerase inhibitor.

In some embodiments, the MAT2A inhibitor and the SPLAM are administered concomitantly. In some embodiments, the MAT2A inhibitor and the SPLAM are administered sequentially. In some embodiments, the MAT2A inhibitor is administered prior to the administration of the SPLAM. In some embodiments, the MAT2A inhibitor is administered after the administration of the SPLAM.

In some embodiments, the MAT2A inhibitor and the KIF18 inhibitor are administered concomitantly. In some embodiments, the MAT2A inhibitor and the KIF18 inhibitor are administered sequentially. In some embodiments, the MAT2A inhibitor is administered prior to the administration of the KIF18 inhibitor. In some embodiments, the MAT2A inhibitor is administered after the administration of the KIF18 inhibitor.

In yet another embodiment, the method comprises administering to the subject in need thereof a MAT2A inhibitor.

In still another embodiment, the method comprises administering to the subject in need thereof a topoisomerase inhibitor, a splicing inhibitor sulfonamide (SPLAM), or a KIF18 inhibitor.

In an embodiment, the MAT2A inhibitor, or a pharmaceutically acceptable salt thereof, and the topoisomerase inhibitor, the splicing inhibitor sulfonamide (SPLAM), or the KIF18 inhibitor, or a pharmaceutically acceptable salt thereof, are administered orally. In one embodiment, the MAT2A inhibitor or a pharmaceutically acceptable salt thereof and the topoisomerase inhibitor or a pharmaceutically acceptable salt thereof are each administered orally. In one embodiment, the MAT2A inhibitor or a pharmaceutically acceptable salt thereof and the SPLAM or a pharmaceutically acceptable salt thereof are each administered orally. In one embodiment, the MAT2A inhibitor or a pharmaceutically acceptable salt thereof and the KIF18 inhibitor or a pharmaceutically acceptable salt thereof are each administered orally.

In another embodiment, the cancer to be treated is selected from the group consisting of leukemia, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, anal cancer, stomach cancer, colon cancer, colorectal cancer, soft tissue sarcoma, non-Hodgkin lymphoma, gastric cancer, esophagogastric cancer, esophageal cancer, malignant peripheral nerve sheath tumor, and mesothelioma.

In an aspect, provided herein is a MAT2A inhibitor, or a pharmaceutically acceptable salt thereof, and a topoisomerase inhibitor, a splicing inhibitor sulfonamide (SPLAM), or a KIF18 inhibitor, or a pharmaceutically acceptable salt thereof, for use in therapy.

In an embodiment, the MAT2A inhibitor, or a pharmaceutically acceptable salt thereof, and the topoisomerase inhibitor, the splicing inhibitor sulfonamide (SPLAM), or the KIF18 inhibitor, or a pharmaceutically acceptable salt thereof, are for use in the treatment of cancer in a subject in need thereof.

Exemplary lengths of time associated with the course of the treatment methods disclosed herein include: about one week; about two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years; and about five years.

In an embodiment of the methods, the method involves the administration of a therapeutically effective amount of a combination or composition comprising compounds provided herein, or pharmaceutically acceptable salts thereof, to a subject (including, but not limited to a human or animal) in need of treatment (including a subject identified as in need).

In another embodiment of the methods, the treatment includes co-administering the amount of the MAT2A inhibitor, or a pharmaceutically acceptable salt thereof, and the amount of the topoisomerase inhibitor, the splicing inhibitor sulfonamide (SPLAM), or the KIF18 inhibitor, or a pharmaceutically acceptable salt thereof. In an embodiment, the amount of the MAT2A inhibitor or a pharmaceutically acceptable salt thereof and the amount of the topoisomerase inhibitor, the splicing inhibitor sulfonamide (SPLAM), or the KIF18 inhibitor or a pharmaceutically acceptable salt thereof are in a single formulation or unit dosage form. In still other embodiments, the amount of MAT2A inhibitor or a pharmaceutically acceptable salt thereof and the amount of the topoisomerase inhibitor, the splicing inhibitor sulfonamide (SPLAM), or the KIF18 inhibitor or a pharmaceutically acceptable salt thereof are in a separate formulations or unit dosage forms.

In the foregoing methods, the treatment can include administering the amount of MAT2A inhibitor or a pharmaceutically acceptable salt thereof and the amount of the topoisomerase inhibitor, the splicing inhibitor sulfonamide (SPLAM), or the KIF18 inhibitor or a pharmaceutically acceptable salt thereof at substantially the same time or administering the amount of MAT2A inhibitor or a pharmaceutically acceptable salt thereof and the amount of the topoisomerase inhibitor, the splicing inhibitor sulfonamide (SPLAM), or the KIF18 inhibitor or a pharmaceutically acceptable salt thereof at different times. In some embodiments of the foregoing methods, the amount of MAT2A inhibitor or a pharmaceutically acceptable salt thereof and/or the amount of the topoisomerase inhibitor, the splicing inhibitor sulfonamide (SPLAM), or the KIF18 inhibitor or a pharmaceutically acceptable salt thereof is administered at dosages that would not be effective when one or both of MAT2A inhibitor or a pharmaceutically acceptable salt thereof and the topoisomerase inhibitor, the splicing inhibitor sulfonamide (SPLAM), or the KIF18 inhibitor or a pharmaceutically acceptable salt thereof is administered alone, but which amounts are effective in combination.

### Pharmaceutical Compositions

In an aspect, provided herein is a pharmaceutical composition comprising a MAT2A inhibitor, or a pharmaceutically acceptable salt thereof, a topoisomerase inhibitor, a splicing inhibitor sulfonamide (SPLAM), or a KIF18 inhibitor, or a pharmaceutically acceptable salt thereof, and at least one pharmaceutically acceptable carrier.

In an embodiment, a pharmaceutical composition comprising a therapeutically effective amount of a MAT2A inhibitor or a pharmaceutically acceptable salt thereof, and a second pharmaceutical composition comprising a therapeutically effective amount of a topoisomerase inhibitor, a splicing inhibitor sulfonamide (SPLAM), or a KIF18 inhibitor or a pharmaceutically acceptable salt thereof is provided.

In another aspect, provided herein is a combination product comprising a first pharmaceutical composition comprising a therapeutically effective amount of a MAT2A inhibitor or a pharmaceutically acceptable salt thereof, and a second pharmaceutical composition comprising a therapeutically effective amount of a topoisomerase inhibitor, a splicing inhibitor sulfonamide (SPLAM), or a KIF18 inhibitor or a pharmaceutically acceptable salt thereof.

In some embodiments, the second pharmaceutical composition comprises a topoisomerase inhibitor. In some embodiments, the second pharmaceutical composition comprises a SPLAM. In some embodiments, the second pharmaceutical composition comprises a KIF18 inhibitor.

In an embodiment, the MAT2A inhibitor is a compound or a pharmaceutically acceptable salt thereof of Formula (I):

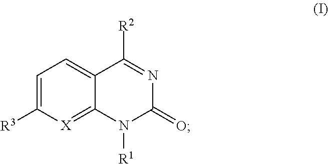

wherein the variables are defined supra.

In another embodiment, the MAT2A inhibitor is selected from the group consisting of a compound from Table 1, or a pharmaceutically acceptable salt thereof.

In another embodiment, the MAT2A inhibitor is Compound A:

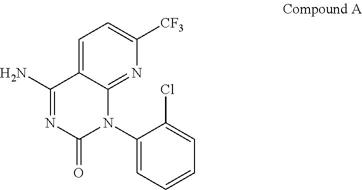 [SMILES: Nc1nc(=O)n(-c2ccccc2Cl)c2nc(C(F)(F)F)ccc12]

or a pharmaceutically acceptable salt thereof.

In another embodiment, the MAT2A inhibitor is Compound A1:

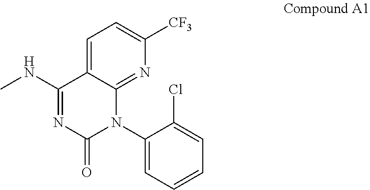 [SMILES: CNc1nc(=O)n(-c2ccccc2Cl)c2nc(C(F)(F)F)ccc12]

or a pharmaceutically acceptable salt thereof.

In an embodiment, the KIF18 inhibitor is sovilnesib. In another embodiment, the SPLAM is indisulam. In yet another embodiment, the SPLAM is selected from the group consisting of E7820, chloroquinoxaline sulfonamide, and tasisulam, or a pharmaceutically acceptable salt thereof. In another embodiment, the topoisomerase inhibitor is selected from the group consisting of 10-hydroxycamptothecin, irinotecan, and topotecan, or a pharmaceutically acceptable salt thereof. In another embodiment, the topoisomerase inhibitor is selected from the group consisting of daunorubicin, doxorubicin, and etoposide, or a pharmaceutically acceptable salt thereof.

In yet another aspect, provided herein is a combination product comprising a first pharmaceutical composition comprising a therapeutically effective amount of Compound A, or a pharmaceutically acceptable salt thereof; and a second pharmaceutical composition comprising a therapeutically effective amount of a topoisomerase inhibitor, a splicing inhibitor sulfonamide (SPLAM), or a KIF18 inhibitor or a pharmaceutically acceptable salt thereof. In some embodiments, the second pharmaceutical composition comprises a topoisomerase inhibitor. In some embodiments, the second pharmaceutical composition comprises a SPLAM. In some embodiments, the second pharmaceutical composition comprises a KIF18 inhibitor.

In an embodiment, the pharmaceutical composition is for use in the treatment of cancer in a patient. In an embodiment, the pharmaceutical composition is for use in the treatment of a solid tumor in a patient. In an embodiment, the pharmaceutical composition is for use in the treatment of a solid malignant tumor in a patient. In still another embodiment, the cancer is MTAP-deficient lung or MTAP-deficient pancreatic cancer, including MTAP-deficient NSCLC or MTAP-deficient PDAC or MTAP-deficient esophageal cancer.

In any one of the embodiments herein, the cancer is a solid tumor or a haematological cancer. In still another embodiment, the cancer is NSCLC, mesothelioma, squamous carcinoma of the head and neck, salivary gland tumors, urothelial cancers, sarcomas, or ovarian cancer. In still another embodiment, the cancer is NSCLC, esophagogastric and pancreatic cancers. In still another embodiment, the cancer is bladder cancer or gastrointestinal cancer. In still another embodiment, the cancer is bladder (urothelial) cancer or gastrointestinal cancer. In an embodiment, the cancer is bladder cancer. In another embodiment, the cancer is urothelial cancer. In yet another embodiment, the cancer is gastrointestinal cancer.

In an embodiment, the pharmaceutical composition is for use in the treatment of mesothelioma in a patient. In an embodiment, the pharmaceutical composition is for use in the treatment of non-small cell lung cancer in a patient. In an embodiment, the pharmaceutical composition is for use in the treatment of nonsquamous non-small cell lung cancer in a patient. In an embodiment, the pharmaceutical composition is for use in the treatment of colon cancer in a patient. In an embodiment, the pharmaceutical composition is for use in the treatment of rectal cancer in a patient. In an embodiment, the pharmaceutical composition is for use in the treatment of colon or rectal adenocarcinoma of the colon or rectum in a patient. In an embodiment, the pharmaceutical composition is for use in the treatment of breast cancer in a patient. In an embodiment, the pharmaceutical composition is for use in the treatment of breast adenocarcinoma in a patient. In an embodiment, the pharmaceutical composition is for use in the treatment of gastric cancer in a patient. In an embodiment, the pharmaceutical composition is for use in the treatment of gastric adenocarcinoma in a patient. In an embodiment, the pharmaceutical composition is for use in the treatment of pancreatic cancer in a patient. In an embodiment, the pharmaceutical composition is for use in the treatment of pancreatic adenocarcinoma in a patient. In an embodiment, the pharmaceutical composition is for use in the treatment of bladder cancer in a patient.

### Administration/Dosage/Formulations

Administration of the combination includes administration of the combination in a single formulation or unit dosage form, administration of the individual agents of the combination concurrently but separately, or administration of the individual agents of the combination sequentially by any suitable route. The dosage of the individual agents of the combination may require more frequent administration of one of the agent(s) as compared to the other agent(s) in the combination. Therefore, to permit appropriate dosing, packaged pharmaceutical products may contain one or more dosage forms that contain the combination of agents, and one or more dosage forms that contain one of the combination of agents, but not the other agent(s) of the combination.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the compounds provided herein are formulated using one or more pharmaceutically acceptable excipients or carriers. In one embodiment, the pharmaceutical compositions provided herein comprise a therapeutically effective amount of a disclosed compound and a pharmaceutically acceptable carrier.

The optimum ratios, individual and combined dosages, and concentrations of the drug compounds that yield efficacy without toxicity are based on the kinetics of the active ingredients' availability to target sites, and are determined using methods known to those of skill in the art.

Routes of administration of any of the compositions discussed herein include oral, nasal, rectal, intravaginal, parenteral, buccal, sublingual or topical. The compounds may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions are not limited to the particular formulations and compositions that are described herein.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

### Kits

In an aspect, the present disclosure provides a kit for treating cancer comprising a MAT2A inhibitor, or a pharmaceutically acceptable salt thereof, and a topoisomerase inhibitor, a splicing inhibitor sulfonamide (SPLAM), or a KIF18 inhibitor or a pharmaceutically acceptable salt thereof.

In certain embodiments, the kit comprises a pharmaceutical product comprising a pharmaceutical composition comprising a MAT2A inhibitor, or a pharmaceutically acceptable salt thereof, and a pharmaceutically acceptable carrier or diluent; and a pharmaceutical composition comprising a topoisomerase inhibitor, a splicing inhibitor sulfonamide (SPLAM), or a KIF18 inhibitor or a pharmaceutically acceptable salt thereof, and a pharmaceutically acceptable carrier or diluent.

In some embodiments, the kit comprises a pharmaceutical composition comprising a MAT2A inhibitor, or a pharmaceutically acceptable salt thereof; a topoisomerase inhibitor, a splicing inhibitor sulfonamide (SPLAM), or a KIF18 inhibitor or a pharmaceutically acceptable salt thereof; and a pharmaceutically acceptable carrier or diluent.

In additional embodiments, pharmaceutical kits are provided. The kit includes a sealed container approved for the storage of pharmaceutical compositions, the container containing one of the above-described pharmaceutical compositions. In some embodiments, the sealed container minimizes the contact of air with the ingredients, e.g. an airless bottle. In other embodiments, the sealed container is a sealed tube. An instruction for the use of the composition and the information about the composition are to be included in the kit.

In a particular embodiment, the compounds of the combination can be dosed on the same schedule, whether by administering a single formulation or unit dosage form containing all of the compounds of the combination, or by administering separate formulations or unit dosage forms of the compounds of the combination. However, some of the compounds used in the combination may be administered more frequently than once per day, or with different frequencies that other compounds in the combination. Therefore, in one embodiment, the kit contains a formulation or unit dosage form containing all of the compounds in the combination of compounds, and an additional formulation or unit dosage form that includes one of the compounds in the combination of agents, with no additional active compound, in a container, with instructions for administering the dosage forms on a fixed schedule.

The kits provided herein include prescribing information, for example, to a patient or health care provider, or as a label in a packaged pharmaceutical formulation. Prescribing information may include for example efficacy, dosage and administration, contraindication and adverse reaction information pertaining to the pharmaceutical formulation.

In all of the foregoing the combination of compounds of the invention can be administered alone, as mixtures, or with additional active agents.

A kit provided herein can be designed for conditions necessary to properly maintain the components housed therein (e.g., refrigeration or freezing). A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.).

Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

### NON-LIMITING EXEMPLARY EMBODIMENTS

### Section A—Non-Limiting Exemplary Embodiments 1 to 112

In further embodiments 1 to 112 below, the present disclosure includes:

Embodiment 1. In Embodiment 1, provided is a method of treating cancer in a subject in need thereof comprising administering to the subject a therapeutically effective amount of a MAT2A inhibitor and administering to the subject a therapeutically effective amount of a KIF18 inhibitor.

Embodiment 2. In Embodiment 2, provided is a method of treating cancer in a subject in need thereof comprising administering to the subject a therapeutically effective amount of a MAT2A inhibitor and, wherein the subject has received treatment with a KIF18 inhibitor.

Embodiment 3. In Embodiment 3, provided is a method of treating cancer in a subject in need thereof comprising administering to the subject a therapeutically effective amount of a MAT2A inhibitor and, wherein the subject is concurrently receiving a KIF18 inhibitor.

Embodiment 4. In Embodiment 4, provided is the method of any one of embodiments 1 to 3, wherein the MAT2A inhibitor is a compound of Formula (I) or a pharmaceutically acceptable salt thereof:

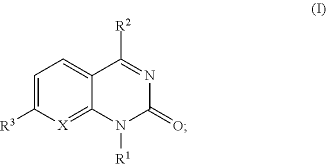

wherein
X is N or CH;
R³ is C₁₋₆ haloalkyl, halo, or C₃₋₆ cycloalkyl;
R² is —NR⁴R⁵;
R⁴ is hydrogen or C₁₋₆ alkyl;
R⁵ is hydrogen, C₁₋₆ alkyl, or C₃₋₆ cycloalkyl; and
R¹ is phenyl, wherein phenyl is substituted with 0-2 halo.

Embodiment 5. In Embodiment 5, provided is the method of any one of Embodiments 1 to 4, wherein the KIF18 inhibitor is a KIF18A inhibitor.

Embodiment 6. In Embodiment 6, provided is the method of any one of Embodiments 1 to 5 wherein the KIF18A inhibitor is sovilnesib (Compound B), or a pharmaceutically acceptable salt thereof.

Embodiment 7. In Embodiment 7, provided is the method of any one of embodiments 1 to 6, wherein the MAT2A inhibitor is administered simultaneously or sequentially with the KIF18 inhibitor.

Embodiment 8. In Embodiment 8, provided is a method of treating cancer in a subject in need thereof comprising administering to the subject a therapeutically effective amount of a MAT2A inhibitor and administering to the subject a therapeutically effective amount of a splicing inhibitor sulfonamide (SPLAM).

Embodiment 9. In Embodiment 9, provided is a method of treating cancer in a subject in need thereof comprising administering to the subject a therapeutically effective amount of a MAT2A inhibitor and, wherein the subject has received treatment with a splicing inhibitor sulfonamide (SPLAM).

Embodiment 10. In Embodiment 10, provided is a method of treating cancer in a subject in need thereof comprising administering to the subject a therapeutically effective amount of a MAT2A inhibitor and, wherein the subject is concurrently receiving a splicing inhibitor sulfonamide (SPLAM).

Embodiment 11. In Embodiment 11, provided is the method of any one of embodiments 8 to 10, wherein the MAT2A inhibitor is a compound of Formula (I) or a pharmaceutically acceptable salt thereof:

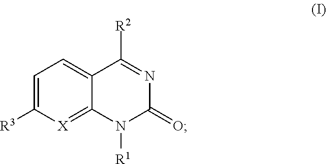

wherein
X is N or CH;
R³ is C₁₋₆ haloalkyl, halo, or C₃₋₆ cycloalkyl;
R² is —NR⁴R⁵;
R⁴ is hydrogen or C₁₋₆ alkyl;
R⁵ is hydrogen, C₁₋₆ alkyl, or C₃₋₆ cycloalkyl; and
R¹ is phenyl, wherein phenyl is substituted with 0-2 halo.

Embodiment 12. In Embodiment 12, provided is the method according to any one of embodiments 8 to 11, wherein the SPLAM is indisulam, or a pharmaceutically acceptable salt thereof.

Embodiment 13. In Embodiment 13, provided is the method according to any one of embodiments 8 to 11, wherein the SPLAM is selected from the group consisting of E7820, chloroquinoxaline sulfonamide, and tasisulam, or a pharmaceutically acceptable salt thereof.

Embodiment 14. In Embodiment 14, provided is the method according to any one of embodiments 8 to 13, wherein the MAT2A inhibitor is administered simultaneously or sequentially with the SPLAM.

Embodiment 15. In Embodiment 15, provided is a method of treating cancer in a subject in need thereof comprising administering to the subject a therapeutically effective amount of a MAT2A inhibitor and administering to the subject a therapeutically effective amount of a topoisomerase inhibitor.

Embodiment 16. In Embodiment 16, provided is a method of treating cancer in a subject in need thereof comprising administering to the subject a therapeutically effective amount of a MAT2A inhibitor and, wherein the subject has received treatment with a topoisomerase inhibitor.

Embodiment 17. In Embodiment 17, provided is a method of treating cancer in a subject in need thereof comprising administering to the subject a therapeutically effective amount of a MAT2A inhibitor and, wherein the subject is concurrently receiving a topoisomerase inhibitor.

Embodiment 18. In Embodiment 18, provided is the method according to any one of embodiments 15 to 17, wherein the MAT2A inhibitor is a compound of Formula (I) or a pharmaceutically acceptable salt thereof:

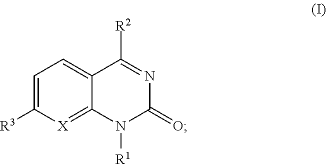

wherein
X is N or CH;
R³ is C₁₋₆ haloalkyl, halo, or C₃₋₆ cycloalkyl;
R² is —NR⁴R⁵;
R⁴ is hydrogen or C₁₋₆ alkyl;
R⁵ is hydrogen, C₁₋₆ alkyl, or C₃₋₆ cycloalkyl; and
R¹ is phenyl, wherein phenyl is substituted with 0-2 halo.

Embodiment 19. In Embodiment 19, provided is the method according to any one of embodiments 15 to 18, wherein the topoisomerase inhibitor is a Type I topoisomerase inhibitor.

Embodiment 20. In Embodiment 20, provided is the method according to embodiment 19, wherein the Type I topoisomerase inhibitor is selected from the group consisting of 10-hydroxycamptothecin, irinotecan, and topotecan, or a pharmaceutically acceptable salt thereof.

Embodiment 20A. In Embodiment 20A, provided is the method according to embodiment 19, wherein the Type I topoisomerase inhibitor is selected from the group consisting of hexylresorcinol, exatecan, deruxtecan, and belotecan, or a pharmaceutically acceptable salt thereof.

Embodiment 21. In Embodiment 21, provided is the method according to any one of embodiments 15 to 18, wherein the topoisomerase inhibitor is a Type II topoisomerase inhibitor.

Embodiment 22. In Embodiment 22, provided is the method according to embodiment 21, wherein the Type II topoisomerase inhibitor is selected from the group consisting of daunorubicin, doxorubicin, and etoposide, or a pharmaceutically acceptable salt thereof.

Embodiment 23. In Embodiment 23, provided is the method according to any one of embodiments 15 to 22, wherein the MAT2A inhibitor is administered simultaneously or sequentially with the topoisomerase inhibitor.

Embodiment 24. In Embodiment 24, provided is the method according to any one of embodiments 4 to 7, 11 to 14, and 18 to 23, wherein X is N.

Embodiment 25. In Embodiment 25, provided is the method according to any one of embodiments 4 to 7, 11 to 14, and 18 to 23, wherein X is CH.

Embodiment 26. In Embodiment 26, provided is the method according to any one of embodiments 4 to 7, 11 to 14, and 18 to 25, wherein R⁴ is hydrogen and R⁵ is hydrogen or C₁₋₃ alkyl.

Embodiment 27. In Embodiment 27, provided is the method according to any one of embodiments 4 to 7, 11 to 14, and 18 to 26, wherein R⁵ is hydrogen or methyl.

Embodiment 28. In Embodiment 28, provided is the method according to any one of embodiments 4 to 7, 11 to 14, and 18 to 27, wherein R¹ is phenyl substituted with chloro.

Embodiment 29. In Embodiment 29, provided is the method according to any one of embodiments 4 to 7, 11 to 14, and 18 to 28, wherein R³ is C₁₋₃ haloalkyl or halo.

Embodiment 30. In Embodiment 30, provided is the method according to any one of embodiments 4 to 7, 11 to 14, and 18 to 29, wherein R³ is trifluoromethyl or chloro.

Embodiment 31. In Embodiment 31, provided is the method according to any one of embodiments 4 to 7, 11 to 14, and 18 to 28, wherein R³ is cyclopropyl.

Embodiment 32. In Embodiment 32, provided is the method according to any one of embodiments 1 to 31, wherein the MAT2A inhibitor is selected from the group consisting of a compound from Table 1, or a pharmaceutically acceptable salt thereof.

Embodiment 33. In Embodiment 33, provided is the method according to any one of embodiments 1 to 32, wherein the MAT2A inhibitor is Compound A or a pharmaceutically acceptable salt thereof.

Embodiment 34. In Embodiment 34, provided is the method according to any one of embodiments 1 to 32, wherein the MAT2A inhibitor is Compound A1 or a pharmaceutically acceptable salt thereof.

Embodiment 35. In Embodiment 35, provided is the method according to any one of embodiments 1 to 34, wherein the cancer is selected from the group consisting of leukemia, glioma, lung cancer, esophageal cancer, MTAP-deficient pancreatic ductal adenocarcinoma (PDAC), melanoma, pancreatic, non-small cell lung cancer, bladder cancer, astrocytoma, osteosarcoma, head and neck cancer, myxoid chondrosarcoma, ovarian cancer, endometrial cancer, breast cancer, anal cancer, stomach cancer, colon cancer, colorectal cancer, soft tissue sarcoma, non-Hodgkin lymphoma, gastric cancer, esophagogastric cancer, oesophageal cancer, malignant peripheral nerve sheath tumor, mesothelioma, salivary gland tumors, urothelial cancers, gastrointestinal cancer, and sarcomas.

Embodiment 36. In Embodiment 36, provided is the method according to any one of embodiments 1 to 35, wherein the cancer is a solid tumor or a hematological cancer.

Embodiment 37. In Embodiment 37, provided is the method according to any one of embodiments 1 to 36, wherein the cancer is a solid tumor.

Embodiment 38. In Embodiment 38, provided is the method according to any one of embodiments 1 to 37, wherein the cancer is a solid malignant tumor.

Embodiment 38A. In Embodiment 38A, provided is the method according to any one of embodiments 1 to 37, wherein the cancer is characterized by a reduction or absence of MTAP gene expression, an absence of MTAP gene, a reduced function of MTAP protein, a reduced level or absence of MTAP protein, a MTA accumulation, or a combination thereof.

Embodiment 39. In Embodiment 39, provided is the method of any one of embodiments 1 to 38 and 38A, wherein the cancer is characterized by MTAP gene deletion.

Embodiment 40. In Embodiment 40, provided is the use of a MAT2A inhibitor in the manufacture of a medicament for treating cancer, wherein the MAT2A inhibitor is administered simultaneously or sequentially with a KIF18 inhibitor.

Embodiment 41. In Embodiment 41, the KIF18 inhibitor of embodiment 40, wherein the KIF18 inhibitor is a KIF18A inhibitor.

Embodiment 42. In Embodiment 42, the KIF18A inhibitor of embodiment 41, wherein the KIF18A inhibitor is sovilnesib (Compound B), or a pharmaceutically acceptable salt thereof.

Embodiment 43. In Embodiment 43, provided is the use of a MAT2A inhibitor in the manufacture of a medicament for treating cancer, wherein the MAT2A inhibitor is administered simultaneously or sequentially with a SPLAM.

Embodiment 44. In Embodiment 44, the SPLAM of embodiment 43, wherein the SPLAM is indisulam, or a pharmaceutically acceptable salt thereof.

Embodiment 45. In Embodiment 45, the SPLAM of embodiment 43, wherein the SPLAM is selected from the group consisting of E7820, chloroquinoxaline sulfonamide, and tasisulam, or a pharmaceutically acceptable salt thereof.

Embodiment 46. In Embodiment 46, provided s the use of a MAT2A inhibitor in the manufacture of a medicament for treating cancer, wherein the MAT2A inhibitor is to be administered simultaneously or sequentially with a topoisomerase inhibitor.

Embodiment 47. In Embodiment 47, the topoisomerase inhibitor of embodiment 46 wherein the topoisomerase inhibitor is a Type I topoisomerase inhibitor.

Embodiment 48. In Embodiment 48, the Type I topoisomerase inhibitor of embodiment 47, wherein the Type I topoisomerase inhibitor is selected from the group consisting of 10-hydroxycamptothecin, irinotecan, and topotecan, or a pharmaceutically acceptable salt thereof.

Embodiment 48A. In Embodiment 48A, provided is the use according to embodiment 47, wherein the Type I topoisomerase inhibitor is selected from the group consisting of hexylresorcinol, exatecan, deruxtecan, and belotecan, or a pharmaceutically acceptable salt thereof.

Embodiment 49. In Embodiment 49, the topoisomerase inhibitor of embodiment 48, wherein the topoisomerase inhibitor is a Type II topoisomerase inhibitor.

Embodiment 50. In Embodiment 50, the Type II topoisomerase inhibitor of embodiment 49, wherein the Type II topoisomerase inhibitor is selected from the group consisting of daunorubicin, doxorubicin, and etoposide, or a pharmaceutically acceptable salt thereof.

Embodiment 51. In Embodiment 51, provided is the use of any one of embodiments 40 to 50, wherein the MAT2A inhibitor is a compound of Formula (I), or a pharmaceutically acceptable salt thereof:

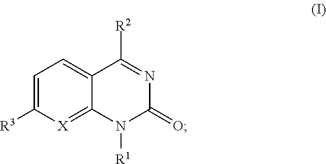

wherein
X is N or CH;
R³ is C₁₋₆ haloalkyl, halo, or C₃₋₆ cycloalkyl;
R² is —NR⁴R⁵;
R⁴ is hydrogen or C₁₋₆ alkyl;
R⁵ is hydrogen, C₁₋₆ alkyl, or C₃₋₆ cycloalkyl; and
R¹ is phenyl, wherein phenyl is substituted with 0-2 halo.

Embodiment 52. In Embodiment 52, provided is the use of MAT2A inhibitor of embodiment 51, wherein the MAT2A inhibitor is a compound of Formula (I), or a pharmaceutically acceptable salt thereof as defined in any one of embodiments 24 to 31.

Embodiment 53. In Embodiment 53, provided is the use of MAT2A inhibitor of any one of embodiments 40 to 52, wherein the MAT2A inhibitor is selected from the group consisting of a compound from Table 1, or a pharmaceutically acceptable salt thereof.

Embodiment 54. In Embodiment 54, provided is the use of MAT2A inhibitor of any one of embodiments 40 to 53, wherein the MAT2A inhibitor is Compound A or a pharmaceutically acceptable salt thereof.

Embodiment 55. In Embodiment 55, provided is the use of MAT2A inhibitor of any one of embodiments 40 to 53, wherein the MAT2A inhibitor is Compound A1, or a pharmaceutically acceptable salt thereof.

Embodiment 56. In Embodiment 56, provided is a MAT2A inhibitor for use in treating cancer, wherein the MAT2A inhibitor is to be administered simultaneously or sequentially with a KIF18 inhibitor.

Embodiment 57. In Embodiment 57, the KIF18 inhibitor of embodiment 57, wherein the KIF18 inhibitor is a KIF18A inhibitor.

Embodiment 58. In Embodiment 58, the KIF18A inhibitor of embodiment 57, wherein the KIF18A inhibitor is sovilnesib (Compound B), or a pharmaceutically acceptable salt thereof.

Embodiment 59. In Embodiment 59, provided is a MAT2A inhibitor for use in treating cancer, wherein the MAT2A inhibitor is to be administered simultaneously or sequentially with a SPLAM.

Embodiment 60. In Embodiment 60, the SPLAM of embodiment 59, wherein the SPLAM is indisulam, or a pharmaceutically acceptable salt thereof.

Embodiment 61. In Embodiment 61, the SPLAM of embodiment 59, wherein the SPLAM is selected from the group consisting of E7820, chloroquinoxaline sulfonamide, and tasisulam, or a pharmaceutically acceptable salt thereof.

Embodiment 62. In Embodiment 62, provided is a MAT2A inhibitor for use in treating cancer, wherein the MAT2A inhibitor is to be administered simultaneously or sequentially with a topoisomerase inhibitor.

Embodiment 63. In Embodiment 63, the topoisomerase inhibitor of embodiment 62, wherein the topoisomerase inhibitor is a Type I topoisomerase inhibitor.

Embodiment 64. In Embodiment 64, the Type I topoisomerase inhibitor of embodiment 63, wherein the Type I topoisomerase inhibitor is selected from the group consisting of 10-hydroxycamptothecin, irinotecan, and topotecan, or a pharmaceutically acceptable salt thereof.

Embodiment 64A. In Embodiment 64A, provided is the use according to embodiment 63, wherein the Type I topoisomerase inhibitor is selected from the group consisting of hexylresorcinol, exatecan, deruxtecan, and belotecan, or a pharmaceutically acceptable salt thereof.

Embodiment 65. In Embodiment 65, the topoisomerase inhibitor of embodiment 62, wherein the topoisomerase inhibitor is a Type II topoisomerase inhibitor.

Embodiment 66. In Embodiment 66, the use of Type II topoisomerase inhibitor of embodiment 65, wherein the Type II topoisomerase inhibitor is selected from the group consisting of daunorubicin, doxorubicin, and etoposide, or a pharmaceutically acceptable salt thereof.

Embodiment 67. In Embodiment 67, provided is the use of MAT2A inhibitor of any one of embodiments 56 to 66, wherein the MAT2A inhibitor is a compound of Formula (I), or a pharmaceutically acceptable salt thereof:

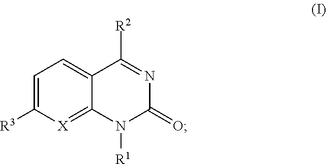

wherein
X is N or CH;
R³ is C₁₋₆ haloalkyl, halo, or C₃₋₆ cycloalkyl;
R² is —NR⁴R⁵;
R⁴ is hydrogen or C₁₋₆ alkyl;
R⁵ is hydrogen, C₁₋₆ alkyl, or C₃₋₆ cycloalkyl; and
R¹ is phenyl, wherein phenyl is substituted with 0-2 halo.

Embodiment 68. In Embodiment 68, provided is the use of MAT2A inhibitor of embodiment 67, wherein the MAT2A inhibitor is a compound of Formula (I), or a pharmaceutically acceptable salt thereof as defined in any one of embodiments 24 to 31.

Embodiment 69. In Embodiment 69, provided is the use of MAT2A inhibitor of any one of embodiments 56 to 68, wherein the MAT2A inhibitor is selected from the group consisting of a compound from Table 1, or a pharmaceutically acceptable salt thereof.

Embodiment 70. In Embodiment 70, provided is the use of MAT2A inhibitor of any one of embodiments 56 to 69, wherein the MAT2A inhibitor is Compound A or a pharmaceutically acceptable salt thereof.

Embodiment 71. In Embodiment 71, provided is the use of MAT2A inhibitor of any one of embodiments 56 to 69, wherein the MAT2A inhibitor is Compound A1, or a pharmaceutically acceptable salt thereof.

Embodiment 72. In Embodiment 72, provided is the use according to any one of embodiments 40 to 71, wherein the cancer is selected from the group consisting of leukemia, glioma, lung cancer, esophageal cancer, MTAP-deficient pancreatic ductal adenocarcinoma (PDAC), melanoma, pancreatic, non-small cell lung cancer, bladder cancer, astrocytoma, osteosarcoma, head and neck cancer, myxoid chondrosarcoma, ovarian cancer, endometrial cancer, breast cancer, anal cancer, stomach cancer, colon cancer, colorectal cancer, soft tissue sarcoma, non-Hodgkin lymphoma, gastric cancer, esophagogastric cancer, malignant peripheral nerve sheath tumor, mesothelioma, salivary gland tumors, urothelial cancers, gastrointestinal cancer, and sarcomas.

Embodiment 73. In Embodiment 73, provided is the use according to any one of embodiments 40 to 72, wherein the cancer is a solid tumor or a hematological cancer.

Embodiment 74. In Embodiment 74, provided is the use according to any one of embodiments 40 to 73, wherein the cancer is a solid tumor.

Embodiment 75. In Embodiment 75, provided is the use according to any one of embodiments 40 to 74, wherein the cancer is a solid malignant tumor.

Embodiment 76. In Embodiment 76, provided is the use according to any one of embodiments 40 to 75, wherein the cancer is characterized by a reduction or absence of MTAP gene expression, an absence of MTAP gene, a reduced function of MTAP protein, a reduced level or absence of MTAP protein, a MTA accumulation, or a combination thereof.

Embodiment 77. In Embodiment 77, provided is the method of any one of embodiments 40 to 76, wherein the cancer is characterized by MTAP gene deletion.

Embodiment 78. In Embodiment 78, provided is a method of inhibiting tumor growth or slowing the rate of tumor growth in a subject with an MTAP deleted cancer, the method comprising administration of a MAT2A inhibitor and a KIF18 inhibitor, a SPLAM, or a topoisomerase inhibitor.

Embodiment 79. In Embodiment 79, provided is the method according to embodiment 78, wherein tumor growth is measured by change in tumor volume from a first time point to a second time point.

Embodiment 80. In Embodiment 80, provided is the method according to embodiment 79, wherein the tumor volume at the second time point shows no increase when compared to the first time point.

Embodiment 81. In Embodiment 81, provided is the method according to embodiment 80, wherein tumor volume decreases between the first time point and the second time point.

Embodiment 82. In Embodiment 82, provided is a method of treating cancer in a subject in need thereof, the method comprising administering to the subject a MAT2A inhibitor and a KIF18 inhibitor, a SPLAM, or a topoisomerase inhibitor, wherein the subject has undergone a previous cancer treatment regimen without a MAT2A inhibitor.

Embodiment 83. In Embodiment 83, provided is the method according to embodiment 82, where treatment with the MAT2A inhibitor and a KIF18 inhibitor, a SPLAM, or a topoisomerase inhibitor decreases the rate of tumor growth when compared to the treatment with the MAT2A inhibitor only for a similar time period.

Embodiment 84. In Embodiment 84, provided is a combination product comprising a MAT2A inhibitor, and an inhibitor selected from the group consisting of KIF18 inhibitor, SPLAM, and a topoisomerase inhibitor.

Embodiment 85. In Embodiment 85, provided is a combination product comprising a first pharmaceutical composition comprising a therapeutically effective amount of a MAT2A inhibitor and a second pharmaceutical composition comprising a therapeutically effective amount of an inhibitor selected from the group consisting of KIF18 inhibitor, SPLAM, and topoisomerase inhibitor.

Embodiment 86. In Embodiment 86, the MAT2A inhibitor of embodiment 84 or 85 is a compound of Formula (I), or a pharmaceutically acceptable salt thereof:

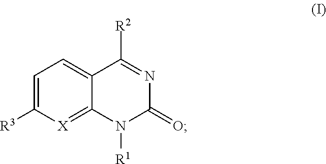

wherein
X is N or CH;
R³ is C₁₋₆ haloalkyl, halo, or C₃₋₆ cycloalkyl;
R² is —NR⁴R⁵;
R⁴ is hydrogen or C₁₋₆ alkyl;
R⁵ is hydrogen, C₁₋₆ alkyl, or C₃₋₆ cycloalkyl; and
R¹ is phenyl, wherein phenyl is substituted with 0-2 halo.

Embodiment 87. In Embodiment 87, the MAT2A inhibitor of embodiment 86 is a compound of Formula (I), or a pharmaceutically acceptable salt thereof as defined in any one of embodiments 24 to 31.

Embodiment 88. In Embodiment 88, the MAT2A inhibitor of any one of embodiments 84 to 87 is selected from the group consisting of the compounds in Table 1, or a pharmaceutically acceptable salt thereof.

Embodiment 89. In Embodiment 89, the MAT2A inhibitor of any one of embodiments 84 to 88 is Compound A or a pharmaceutically acceptable salt thereof.

Embodiment 90. In Embodiment 90, the MAT2A inhibitor of any one of embodiments 84 to 88 is Compound A1 or a pharmaceutically acceptable salt thereof.

Embodiment 91. In Embodiment 91, the KIF18 inhibitor of any one of embodiments 84 to 90 is a KIF18A inhibitor.

Embodiment 92. In Embodiment 92, the KIF18A inhibitor of embodiment 91, wherein the KIF18A inhibitor is sovilnesib (Compound B), or a pharmaceutically acceptable salt thereof.

Embodiment 93. In Embodiment 93, the SPLAM of any one of embodiments 84 to 90, wherein the SPLAM is indisulam, or a pharmaceutically acceptable salt thereof.

Embodiment 94. In Embodiment 94, the SPLAM of any one of embodiments 84 to 90, wherein the SPLAM is selected from the group consisting of E7820, chloroquinoxaline sulfonamide, and tasisulam, or a pharmaceutically acceptable salt thereof.

Embodiment 95. In Embodiment 95, the topoisomerase inhibitor of any one of embodiments 84 to 90, wherein the topoisomerase inhibitor is a Type I topoisomerase inhibitor.

Embodiment 96. In Embodiment 96, the Type I topoisomerase inhibitor of embodiment 95, wherein the Type I topoisomerase inhibitor is selected from the group consisting of 10-hydroxycamptothecin, irinotecan, and topotecan, or a pharmaceutically acceptable salt thereof.

Embodiment 96A. In Embodiment 96A, the Type I topoisomerase inhibitor of embodiment 95, wherein the Type I topoisomerase inhibitor is selected from the group consisting of hexylresorcinol, exatecan, deruxtecan, and belotecan, or a pharmaceutically acceptable salt thereof.

Embodiment 96B. In Embodiment 96B, the Type I topoisomerase inhibitor of embodiment 95, wherein the Type I topoisomerase inhibitor is irinotecan.

Embodiment 97. In Embodiment 97, the topoisomerase inhibitor of any one of embodiments 84 to 90, wherein the topoisomerase inhibitor is a Type II topoisomerase inhibitor.

Embodiment 98. In Embodiment 98, the use Type II topoisomerase inhibitor of embodiment 97, wherein the Type II topoisomerase inhibitor is selected from the group consisting of daunorubicin, doxorubicin, and etoposide, or a pharmaceutically acceptable salt thereof.

Embodiment 99. In Embodiment 99, provided is a method of treating cancer in a subject in need thereof comprising administering to the subject a therapeutically effective amount of a MAT2A inhibitor and administering to the subject a therapeutically effective amount of an antibody drug conjugate (ADC) comprising a topoisomerase inhibitor, wherein the MAT2A inhibitor is a compound of Formula (I) or a pharmaceutically acceptable salt thereof.

Embodiment 100. In Embodiment 100, the ADC comprising the topoisomerase inhibitor is a Type I topoisomerase inhibitor.

Embodiment 101. In Embodiment 101, the ADC comprising the topoisomerase inhibitor is fam-trastuzumab deruxtecan-nxki.

Embodiment 102. In Embodiment 102, the ADC comprising the topoisomerase inhibitor is AZD8205.

Embodiment 103. In Embodiment 103, the ADC comprising the topoisomerase inhibitor is DS-1062.

Embodiment 104. In Embodiment 104, the MAT2A inhibitor of any one of embodiments 99 to 103 is selected from the group consisting of the compounds in Table 1, or a pharmaceutically acceptable salt thereof.

Embodiment 105. In Embodiment 105, the MAT2A inhibitor of any one of embodiments 99 to 104 is Compound A or a pharmaceutically acceptable salt thereof.

Embodiment 106. In Embodiment 106, the MAT2A inhibitor of any one of embodiments 99 to 104 is Compound A1 or a pharmaceutically acceptable salt thereof.

Embodiment 107. In Embodiment 107, provided is the method according to any one of embodiments 99 to 106, wherein the cancer is selected from the group consisting of leukemia, glioma, lung cancer, esophageal cancer, MTAP-deficient pancreatic ductal adenocarcinoma (PDAC), melanoma, pancreatic, non-small cell lung cancer, bladder cancer, astrocytoma, osteosarcoma, head and neck cancer, myxoid chondrosarcoma, ovarian cancer, endometrial cancer, breast cancer, anal cancer, stomach cancer, colon cancer, colorectal cancer, soft tissue sarcoma, non-Hodgkin lymphoma, gastric cancer, esophagogastric cancer, malignant peripheral nerve sheath tumor, mesothelioma, salivary gland tumors, urothelial cancers, gastrointestinal cancer, and sarcomas.

Embodiment 108. In Embodiment 108, provided is the method according to any one of embodiments 99 to 107, wherein the cancer is a solid tumor or a hematological cancer.

Embodiment 109. In Embodiment 109, provided is the method according to any one of embodiments 99 to 108, wherein the cancer is a solid tumor.

Embodiment 110. In Embodiment 110, provided is the method according to any one of embodiments 99 to 109, wherein the cancer is a solid malignant tumor.

Embodiment 111. In Embodiment 111, provided is the method according to any one of embodiments 99 to 110, wherein the cancer is characterized by a reduction or absence of MTAP gene expression, an absence of MTAP gene, a reduced function of MTAP protein, a reduced level or absence of MTAP protein, a MTA accumulation, or a combination thereof.

Embodiment 112. In Embodiment 112, provided is the method of any one of embodiments 1 to 111, wherein the cancer is characterized by MTAP gene deletion.

For the embodiments above, when reference is made to previous embodiments, the reference will include those embodiments having lettered notations or combinations. For example, reference to embodiments 40 to 50 will include embodiments 40, 41, 42, 43, 44, 45, 46, 47, 48, 48A, 49, and 50. Section B—Additional non-limiting exemplary Embodiments 1 to 40

Embodiment 1. In embodiment 1, provided is a method of treating cancer in a subject in need thereof comprising administering to the subject a MAT2A inhibitor and administering to the subject a therapeutically effective amount of a topoisomerase inhibitor, wherein the MAT2A inhibitor is a compound of Formula (I) or a pharmaceutically acceptable salt thereof:

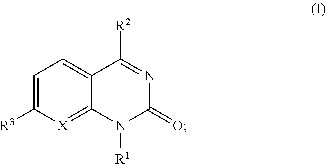

wherein
X is N or CH;
R³ is C₁₋₆ haloalkyl, halo, or C₃₋₆ cycloalkyl;
R² is —NR⁴R⁵;
R⁴ is hydrogen or C₁₋₆ alkyl;
R⁵ is hydrogen, C₁₋₆ alkyl, or C₃₋₆ cycloalkyl; and
R¹ is phenyl, wherein phenyl is substituted with 0-2 halo.

Embodiment 2. In embodiment 2, provided is the method according to embodiment 1, wherein the topoisomerase inhibitor is a Type I topoisomerase inhibitor.

Embodiment 3. In embodiment 3, provided is the method according to embodiment 2, wherein the Type I topoisomerase inhibitor is selected from the group consisting of 10-hydroxycamptothecin, irinotecan, and topotecan, or a pharmaceutically acceptable salt thereof.

Embodiment 4. In embodiment 4, provided is the method according to embodiment 1, wherein the topoisomerase inhibitor is a Type II topoisomerase inhibitor.

Embodiment 5. In embodiment 5, provided is the method according to embodiment 4, wherein the Type II topoisomerase inhibitor is selected from the group consisting of daunorubicin, doxorubicin, and etoposide, or a pharmaceutically acceptable salt thereof.

Embodiment 6. In embodiment 6, provided is the method of any one of embodiments 1 to 5, wherein the MAT2A inhibitor is administered simultaneously or sequentially with the topoisomerase inhibitor.

Embodiment 7. In embodiment 7, provided is a method of treating cancer in a subject in need thereof comprising administering to the subject a MAT2A inhibitor and administering to the subject a therapeutically effective amount of a splicing inhibitor sulfonamide (SPLAM), wherein the MAT2A inhibitor is a compound of Formula (I) or a pharmaceutically acceptable salt thereof:

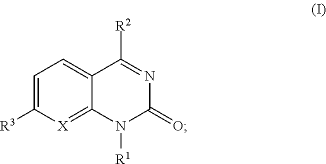

wherein
X is N or CH;
R³ is C₁₋₆ haloalkyl, halo, or C₃₋₆ cycloalkyl;
R² is —NR⁴R⁵;
R⁴ is hydrogen or C₁₋₆ alkyl;
R⁵ is hydrogen, C₁₋₆ alkyl, or C₃₋₆ cycloalkyl; and
R¹ is phenyl, wherein phenyl is substituted with 0-2 halo.

Embodiment 8. In embodiment 8, provided is the method according to embodiment 7, wherein the SPLAM is indisulam, or a pharmaceutically acceptable salt thereof.

Embodiment 9. In embodiment 9, provided is the method according to embodiment 7, wherein the SPLAM is selected from the group consisting of E7820, chloroquinoxaline sulfonamide, and tasisulam, or a pharmaceutically acceptable salt thereof.

Embodiment 10. In embodiment 10, provided is the method according to any one of embodiments 7 to 9, wherein the MAT2A inhibitor is administered simultaneously or sequentially with the SPLAM.

Embodiment 11. In embodiment 11, provided is a method of treating cancer in a subject in need thereof comprising administering to the subject a MAT2A inhibitor and administering to the subject a therapeutically effective amount of a KIF18 inhibitor, wherein the MAT2A inhibitor is a compound of Formula (I) or a pharmaceutically acceptable salt thereof:

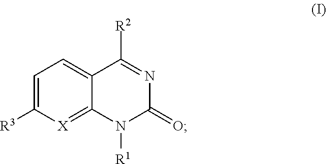

wherein
X is N or CH;
R³ is C₁₋₆ haloalkyl, halo, or C₃₋₆ cycloalkyl;
R² is —NR⁴R⁵;
R⁴ is hydrogen or C₁₋₆ alkyl;
R⁵ is hydrogen, C₁₋₆ alkyl, or C₃₋₆ cycloalkyl; and
R¹ is phenyl, wherein phenyl is substituted with 0-2 halo.

Embodiment 12. In embodiment 12, provided is the method according to embodiment 11, wherein the KIF18 inhibitor is a KIF18A inhibitor.

Embodiment 13. In embodiment 13, provided is the method according to embodiment 12, wherein the KIF18A inhibitor is sovilnesib, or a pharmaceutically acceptable salt thereof.

Embodiment 14. In embodiment 14, provided is the method of any one of embodiments 11B to 13B, wherein the MAT2A inhibitor is administered simultaneously or sequentially with the KIF18 inhibitor.

Embodiment 15. In embodiment 15, provided is The method according to any one of embodiments 1 to 14, wherein X is N.

Embodiment 16. In embodiment 16, provided is the method according to any one of embodiments 1 to 14, wherein X is CH.

Embodiment 17. In embodiment 17, provided is the method according to any one of embodiments 1 to 16, wherein R⁴ is hydrogen and R⁵ is hydrogen or C₁-3 alkyl.

Embodiment 18. In embodiment 18, provided is the method according to any one of embodiments 1 to 17, wherein R⁵ is hydrogen or methyl.

Embodiment 19. In embodiment 19, provided is the method according to any one of embodiments 1 to 18, wherein R¹ is phenyl substituted with chloro.

Embodiment 20. In embodiment 20, provided is the method according to any one of embodiments 1 to 19, wherein R³ is C₁₋₃ haloalkyl or halo.

Embodiment 21. In embodiment 21, provided is the method according to any one of embodiments 1 to 20, wherein R³ is trifluoromethyl or chloro.

Embodiment 22. In embodiment 22, provided is the method according to any one of embodiments 1 to 20, wherein R³ is cyclopropyl.

Embodiment 23. In embodiment 23, provided is the method according to any one of embodiments 1 to 22, wherein the MAT2A inhibitor is selected from the group consisting of a compound from Table 1, or a pharmaceutically acceptable salt thereof.

Embodiment 24. In embodiment 24, provided is the method according to any one of embodiments 1 to 23, wherein the MAT2A inhibitor is Compound A:

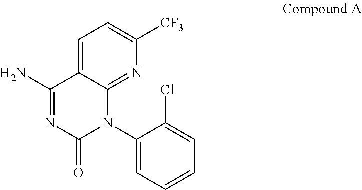 [SMILES: Nc1nc(=O)n(-c2ccccc2Cl)c2nc(C(F)(F)F)ccc12]

or a pharmaceutically acceptable salt thereof.

Embodiment 25. In embodiment 25, provided is the method according to any one of embodiments 1 to 23, wherein the MAT2A inhibitor is Compound A1:

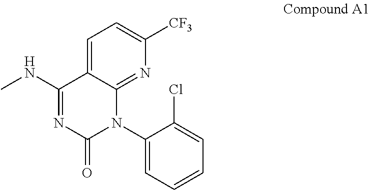 [SMILES: CNc1nc(=O)n(-c2ccccc2Cl)c2nc(C(F)(F)F)ccc12]

or a pharmaceutically acceptable salt thereof.

Embodiment 26. In embodiment 26, provided is the method according to any one of embodiments 1 to 25, wherein the cancer is selected from the group consisting of leukemia, glioma, lung cancer, esophageal cancer, MTAP-deficient pancreatic ductal adenocarcinoma (PDAC), melanoma, pancreatic, non-small cell lung cancer, bladder cancer, astrocytoma, osteosarcoma, head and neck cancer, myxoid chondrosarcoma, ovarian cancer, endometrial cancer, breast cancer, anal cancer, stomach cancer, colon cancer, colorectal cancer, soft tissue sarcoma, non-Hodgkin lymphoma, gastric cancer, esophagogastric cancer, oesophageal cancer, malignant peripheral nerve sheath tumor, mesothelioma, salivary gland tumors, urothelial cancers, gastrointestinal cancer, and sarcomas.

Embodiment 27. In embodiment 27, provided is the method according to any one of embodiments 1 to 26, wherein the cancer is a solid tumor or a hematological cancer.

Embodiment 28. In embodiment 28, provided is the method according to any one of embodiments 1 to 27, wherein the cancer is a solid, malignant tumor.

Embodiment 29. In embodiment 29, provided is the method according to claim any one of embodiments 1 to 28, wherein the cancer is characterized by a reduction or absence of MTAP gene expression, an absence of MTAP gene, a reduced function of MTAP protein, a reduced level or absence of MTAP protein, a MTA accumulation, or a combination thereof.

Embodiment 30. In embodiment 30, provided is use of a MAT2A inhibitor in the manufacture of a medicament for treating cancer, wherein the MAT2A inhibitor is administered with a topoisomerase inhibitor.

Embodiment 31. In embodiment 31, provided is use of a MAT2A inhibitor in the manufacture of a medicament for treating cancer, wherein the MAT2A inhibitor is administered with a SPLAM.

Embodiment 32. In embodiment 32, provided is use of a MAT2A inhibitor in the manufacture of a medicament for treating cancer, wherein the MAT2A inhibitor is administered with a KIF18 inhibitor.

Embodiment 33. In embodiment 33, provided is the use of any one of embodiments 30 to 32, wherein the MAT2A inhibitor is a compound of Formula (I), or a pharmaceutically acceptable salt thereof.

Embodiment 34. In embodiment 34, provided is the use of embodiment 33B, wherein the MAT2A inhibitor is Compound A or a pharmaceutically acceptable salt thereof, or Compound A1, or a pharmaceutically acceptable salt thereof.

Embodiment 35. In embodiment 35, provided is a method of inhibiting tumor growth or slowing the rate of tumor growth in a subject with an MTAP deleted cancer, the method comprising administration of a MAT2A inhibitor and a topoisomerase inhibitor, a SPLAM, or a KIF18 inhibitor.

Embodiment 36. In embodiment 36, provided is The method according to embodiment 35, wherein tumor growth is measured by change in tumor volume from a first time point to a second time point.

Embodiment 37. In embodiment 37, provided is the method according to embodiment 36, wherein the tumor volume at the second time point shows no increase when compared to the first time point.

Embodiment 38. In embodiment 38, provided is the method according to embodiment 37, wherein tumor volume decreases between the first time point and the second time point.

Embodiment 39. In embodiment 39, provided is a method of treating cancer in a subject in need thereof, the method comprising administering to the subject a MAT2A inhibitor and a topoisomerase inhibitor, a SPLAM, or a KIF18 inhibitor, wherein the subject has undergone a previous cancer treatment regimen without a MAT2A inhibitor.

Embodiment 40. In embodiment 40, provided is the method according to embodiment 39, where treatment with the MAT2A inhibitor and a topoisomerase inhibitor, a SPLAM, or a KIF18 inhibitor decreases the rate of tumor growth when compared to the treatment with the MAT2A inhibitor only for a similar time period.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure.

However, they are in no way a limitation of the teachings of the present disclosure as set forth.

### EXAMPLES

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Example 1: In Vitro Synergistic Growth Inhibition with Equimolar Combinations of a MAT2A Inhibitor and a KIF18A Inhibitor

### Materials and Methods

A 10-day proliferation assay was performed in a panel of 5 MTAP-deficient and 2 wild type non-small cell lung and bladder cancer cell lines. Optimal cell seeding for all cell lines was determined by assessing the growth over a range of seeding densities in a 384-well format to identify conditions that permitted proliferation for 10 days. Cells were then plated at the optimal seeding density and treated with 20-point, two-fold dilution series of Compound A, sovilnesib (“Compound B”), or an equimolar combination of Compound A and Compound B. Concentrations tested for Compound A and Compound B alone or in combination ranged from 0.038 nM to 20,000 nM. A plate of cells was harvested at the time of compound addition to quantify the number of cells at the start of the combination (T₀). For cell quantification, the harvested cells were lysed with Promega CellTiter-Glo (CTG) reagent according to the manufacturer's protocol and the chemiluminescent signal was detected on a Synergy Neo plate reader. CTG estimates cell number through detection of cellular ATP levels. Cells were incubated with the drug combinations at 37° C. with 5% CO₂ for 10 days. Cells were then lysed with CTG and the chemiluminescent signal was measured.

### Analysis

CTG values obtained after the 10-day treatment were background subtracted and expressed as a percent of the T₀ value. Data were plotted against compound concentration and fit with a four-parameter equation to generate dose response curves for each single agent and the equimolar combination. Growth ICx (gICx) values ranging from gIC30 to gIC100 were interpolated from the fitted curves. Synergistic growth inhibition was assessed by determination of Combination Index (CI) values at these various points across the titration using the mutually non-exclusive equation [Chou, 1983; Chou, 1981] shown below where A equals the gIC value of Compound A, B equals the gICx value of Compound B, and A+B or B+A equals the gICx value of the equimolar combination. A Combination Index value less than 1.0 was considered synergistic.

$$CI = \frac{{gIC}_{x} of ( A + B )}{{gIC}_{x} of A} + \frac{{gIC}_{x} of ( B + A )}{{gIC}_{x} of B} + \frac{( {gIC}_{x} of ( A + B ) ) ( {gIC}_{x} of ( B + A ) )}{( {gIC}_{x} of A ) ( {gIC}_{x} of B )}$$

### Results

Synergistic growth inhibition was observed with Compound A in combination with Compound B at multiple gICx points along the combination titration in 5/5 MTAP-deficicient cell lines (Tables 3A-3E). Combination synergy was also observed in 1/2 wild type cell lines (Tables 4A-4B). While considered synergistic, the Combination Index values in the wild type TCCSUP cell line were ≥0.7. In comparison to the wild type cell lines, the MTAP-deficient cell lines exhibited greater sensitivity to the combination where Combination Index values were as low as 0.01.

Tables 3A-3E: Synergistic Growth Inhibition with Equimolar Combinations of Compound A and Compound B Across Five MTAP-deficient Cell Lines

Synergy was measured for equimolar combinations of Compound A and Compound B utilizing the mutually non-exclusive Combination Index equation across gICx values ranging from gIC30-gIC100. Combination Index values are shown for: NCI-H838 (3A), SW900 (3B), RT112/84 (3C), UMUC5 (3D), and UMUC11 (3E).

TABLE 3A
NCI-H838

| | Cmpd A | Cmpd B | 1:1 Combo | |
|---|---|---|---|---|
| | (nM) | (nM) | (nM) | Combination Index |
| gIC30 | 52 | 735 | 36 | 0.9 |
| gIC40 | 90 | 1476 | 47 | 0.7 |
| gIC50 | 208 | 12472 | 61 | 0.3 |
| gIC60 | 20000 | 14279 | 81 | 0.01 |
| gIC70 | 20000 | 16962 | 113 | 0.01 |
| gIC80 | 20000 | 20000 | 182 | 0.02 |
| gIC90 | 20000 | 20000 | 518 | 0.1 |
| gIC100 | 20000 | 20000 | 20000 | 3.0 |

TABLE 3B
SW900

| | Cmpd A | Cmpd B | 1:1 Combo | |
|---|---|---|---|---|
| | (nM) | (nM) | (nM) | Combination Index |
| gIC30 | 20000 | 5477 | 795 | 0.2 |
| gIC40 | 20000 | 8546 | 1330 | 0.2 |
| gIC50 | 20000 | 12252 | 2207 | 0.3 |
| gIC60 | 20000 | 16589 | 3846 | 0.5 |
| gIC70 | 20000 | 19918 | 7974 | 0.96 |
| gIC80 | 20000 | 20000 | 17854 | 2.6 |
| gIC90 | 20000 | 20000 | 20000 | 3.0 |
| gIC100 | 20000 | 20000 | 20000 | 3.0 |

TABLE 3C
RT112/84

| | Cmpd A | Cmpd B | 1:1 Combo | |
|---|---|---|---|---|
| | (nM) | (nM) | (nM) | Combination Index |
| gIC30 | 277 | 150 | 84 | 1.0 |
| gIC40 | 854 | 170 | 110 | 0.9 |
| gIC50 | 2997 | 192 | 141 | 0.8 |
| gIC60 | 16002 | 222 | 183 | 0.9 |
| gIC70 | 20000 | 273 | 242 | 0.96 |
| gIC80 | 20000 | 10330 | 341 | 0.3 |
| gIC90 | 20000 | 20000 | 569 | 0.1 |
| gIC100 | 20000 | 20000 | 11394 | 1.6 |

TABLE 3D
UMUC5

| | Cmpd A | Cmpd B | 1:1 Combo | |
|---|---|---|---|---|
| | (nM) | (nM) | (nM) | Combination Index |
| gIC30 | 357 | 170 | 71 | 0.7 |
| gIC40 | 1349 | 194 | 83 | 0.5 |
| gIC50 | 5163 | 221 | 97 | 0.5 |
| gIC60 | 19400 | 253 | 112 | 0.5 |
| gIC70 | 20000 | 296 | 133 | 0.5 |
| gIC80 | 20000 | 370 | 162 | 0.5 |
| gIC90 | 20000 | 662 | 220 | 0.3 |
| gIC100 | 20000 | 20000 | 20000 | 3.0 |

TABLE 3E
UMUC11

| | Cmpd A | Cmpd B | 1:1 Combo | |
|---|---|---|---|---|
| | (nM) | (nM) | (nM) | Combination Index |
| gIC30 | 20000 | 2199 | 174 | 0.1 |
| gIC40 | 20000 | 3485 | 287 | 0.1 |
| gIC50 | 20000 | 5166 | 474 | 0.1 |
| gIC60 | 20000 | 7300 | 810 | 0.2 |
| gIC70 | 20000 | 9950 | 1522 | 0.2 |
| gIC80 | 20000 | 13185 | 3639 | 0.5 |
| gIC90 | 20000 | 17077 | 18037 | 2.9 |
| gIC100 | 20000 | 20000 | 20000 | 3.0 |

Tables 4A-4B: Synergistic Growth Inhibition with Equimolar Combinations of Compound A and Compound B Across Two Wild Type Cell Lines

Synergy was measured for equimolar combinations of Compound A and Compound B utilizing the mutually non-exclusive Combination Index equation across gICx values ranging from gIC30-gIC100. Combination Index values are shown for: NCI-H520 (4A) and TCCSUP (41B).

TABLE 4A
NCI-H520

| | Cmpd A | Cmpd B | 1:1 Combo | |
|---|---|---|---|---|
| | (nM) | (nM) | (nM) | Combination Index |
| gIC30 | 20000 | 2814 | 2877 | 1.3 |
| gIC40 | 20000 | 4163 | 3939 | 1.3 |
| gIC50 | 20000 | 5798 | 5210 | 1.4 |
| gIC60 | 20000 | 7731 | 6742 | 1.5 |
| gIC70 | 20000 | 9976 | 8602 | 1.7 |
| gIC80 | 20000 | 12547 | 10889 | 1.9 |
| gIC90 | 20000 | 15458 | 13745 | 2.2 |
| gIC100 | 20000 | 18724 | 17385 | 2.6 |

TABLE 4B
TCCSUP

| | Cmpd A | Cmpd B | 1:1 Combo | |
|---|---|---|---|---|
| | (nM) | (nM) | (nM) | Combination Index |
| gIC30 | 20000 | 91 | 69 | 0.8 |
| gIC40 | 20000 | 107 | 82 | 0.8 |
| gIC50 | 20000 | 126 | 96 | 0.8 |
| gIC60 | 20000 | 150 | 114 | 0.8 |
| gIC70 | 20000 | 183 | 139 | 0.8 |
| gIC80 | 20000 | 240 | 179 | 0.8 |
| gIC90 | 20000 | 405 | 274 | 0.7 |
| gIC100 | 20000 | 20000 | 20000 | 3.0 |

### Example 2: In Vitro Synergistic Growth Inhibition with a KIF18A Inhibitor in Combination with a Fixed Concentration of a MAT2A Inhibitor

### Materials and Methods

A 10-day proliferation assay was performed in a panel of 5 MTAP-deficient and 2 wild type non-small cell lung and bladder cancer cell lines. Optimal cell seeding for all cell lines was determined by assessing the growth over a range of seeding densities in a 384-well format to identify conditions that permitted proliferation for 10 days. Cells were then plated at the optimal seeding density and treated with 20-point, two-fold dilution series of Compound B in combination with a 1000 nM fixed concentration of Compound A. Single agent titrations for Compound A and Compound B were included for comparison. Concentrations tested for Compound A and Compound B alone or in combination ranged from 0.038 nM to 20,000 nM. A plate of cells was harvested at the time of compound addition to quantify the number of cells at the start of the combination (T₀). For cell quantification, the harvested cells were lysed with Promega CellTiter-Glo (CTG) reagent according to the manufacturer's protocol and the chemiluminescent signal was detected as described in Example 1 above. Cells were incubated with the drug combinations at 37° C. with 5% CO₂ for 10 days. Cells were then lysed with CTG and the chemiluminescent signal was measured.

### Analysis

CTG values obtained after the 10-day treatment were background subtracted and expressed as a percent of the T₀ value set at 100%. Data were plotted against compound concentration and fit with a four-parameter equation to generate single agent dose response curves. Percent inhibition was determined for each combination and single agent concentration compared to a DMSO control. In addition, the overall net growth or death of the cells was calculated and reported as a growth death index (GDI) value on a scale of −100 to 100 with the midpoint (0) being the number of cells at the beginning of the combination (T₀).

Synergistic growth inhibition was assessed using the Bliss Independence model for each point of the Compound B titration in combination with 1000 nM Compound A. The observed growth inhibition at each point was compared to the predicted inhibition based on the additive activity of each single agent. Predicted inhibition was calculated using (Ea+Eb)−(Ea*Eb) where E equals the effect (inhibition) of each single agent (a and b). A difference between the observed inhibition percentage and the predicted inhibition percentage greater than 10% was considered synergistic. A Bliss score was only calculated for combinations eliciting >20% growth inhibition (GDI value <80). Since Bliss is calculated based on growth inhibition, scores within cytotoxic dose ranges may not accurately reflect synergistic cytotoxicity.

### Results

Synergistic growth inhibition was observed with Compound B in combination with 1000 nM Compound A across numerous points of the titration as assessed by Bliss Independence in 5/5 MTAP-deficient cell lines (Tables 5A-5E). Due to the sharp dose response curves observed with single agent Compound B, the window available for observation of combination synergy within this assay design was narrow for some cell lines.

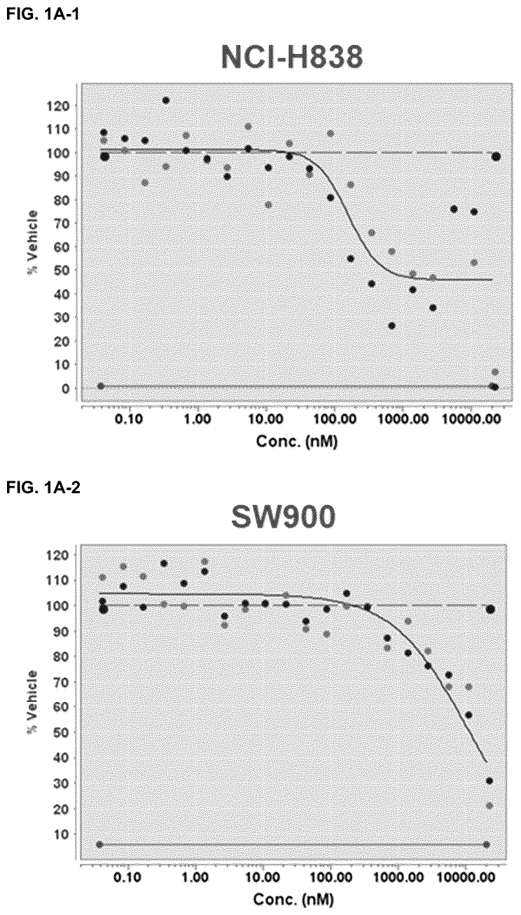

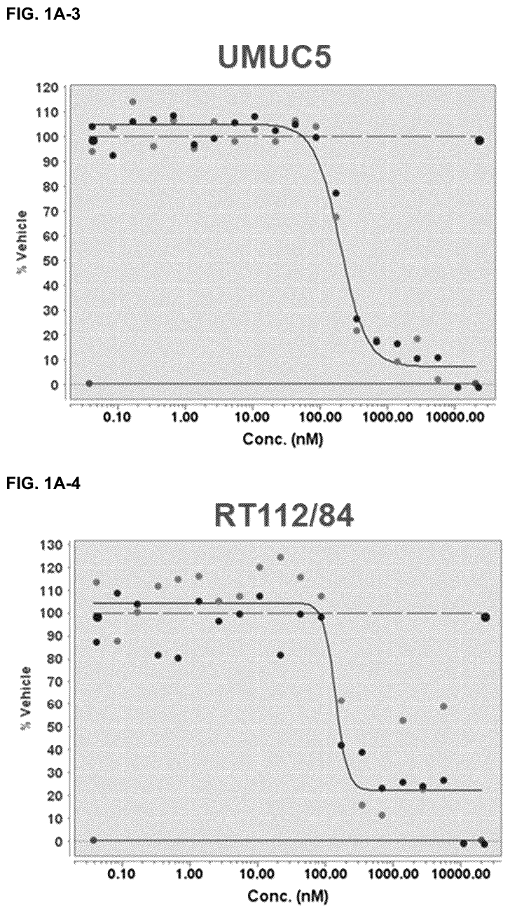

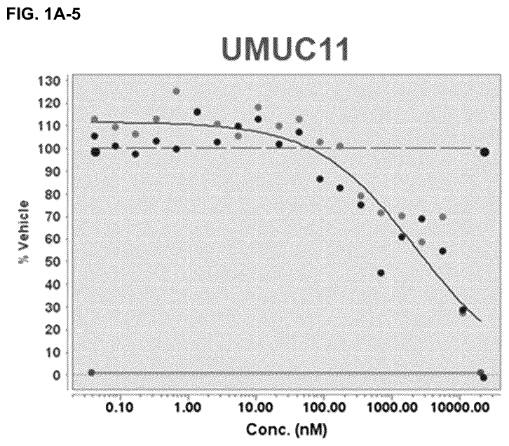

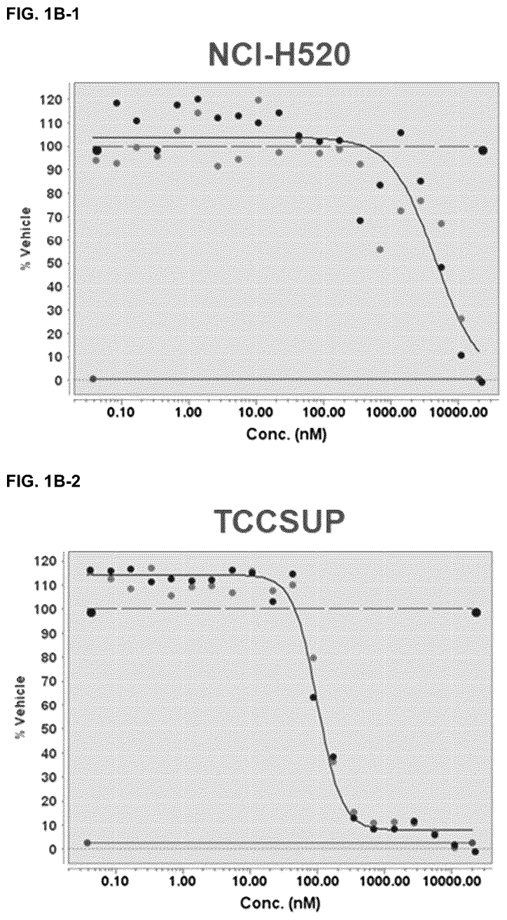

For example, 5000 nM single agent Compound B is sufficient to maximally inhibit cell growth in the UMUC5 cell line (FIG. 1A-3). Therefore, the dose range in which combination synergy can be observed in UMUC5 is below the 5000 nM Compound B dose. Despite this limited window, combination synergy was observed at doses below that needed for maximal single agent inhibition in 5/5 MTAP-deficient cell lines. In these cell lines, synergistic growth inhibition was observed across a broad dose range of Compound B in combination with 1000 nM Compound A.

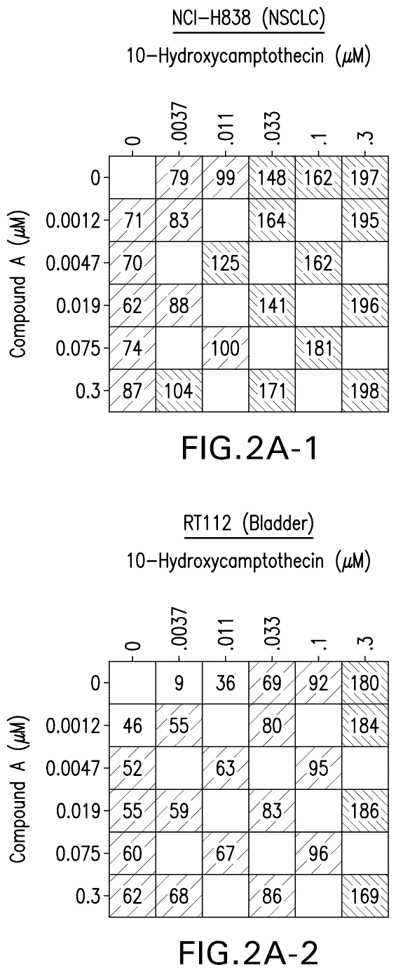

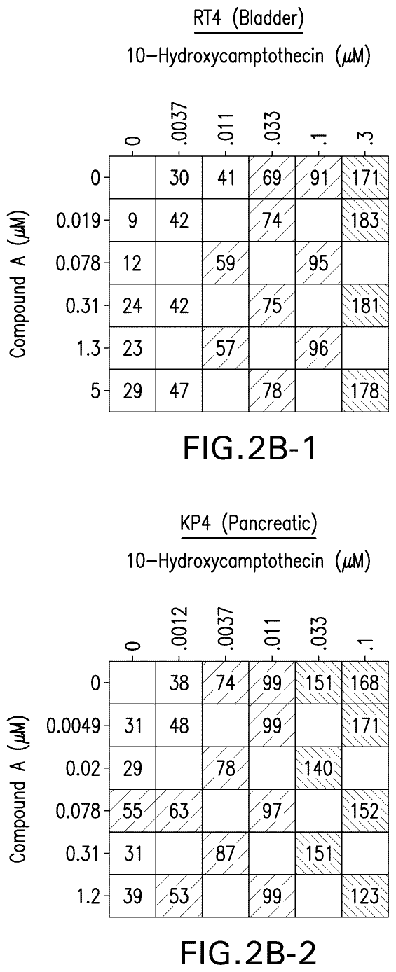

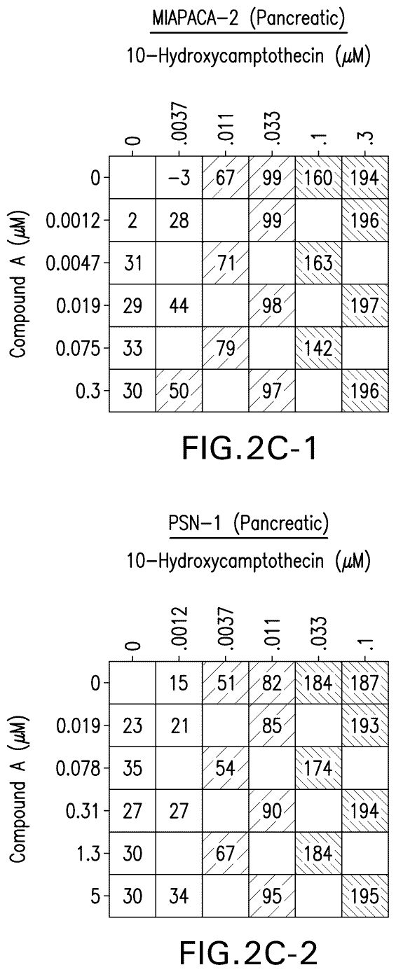

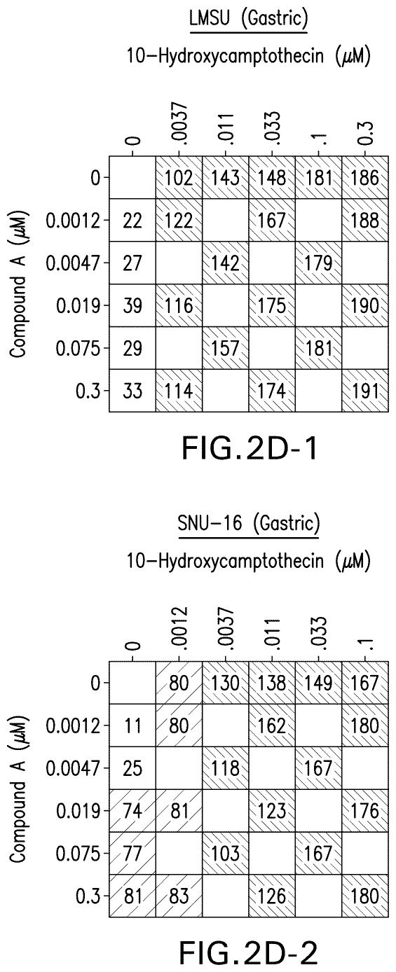

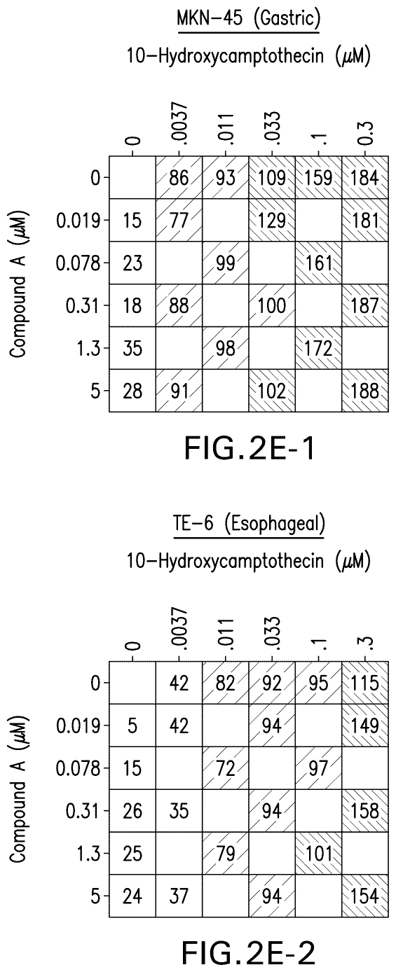

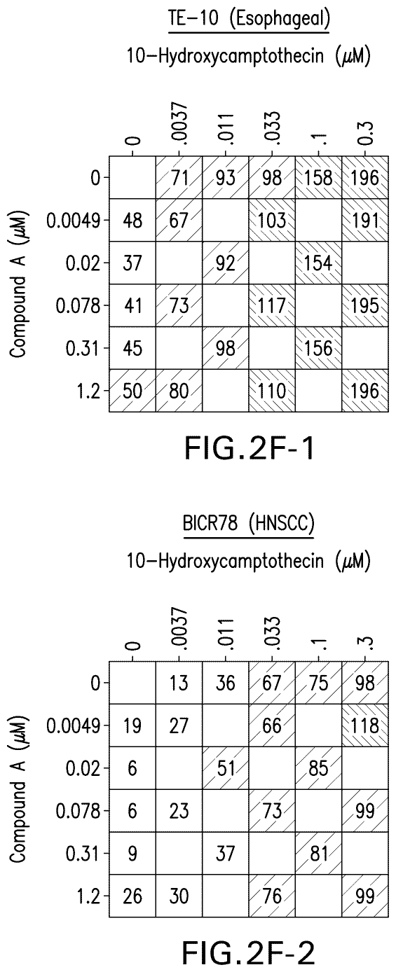

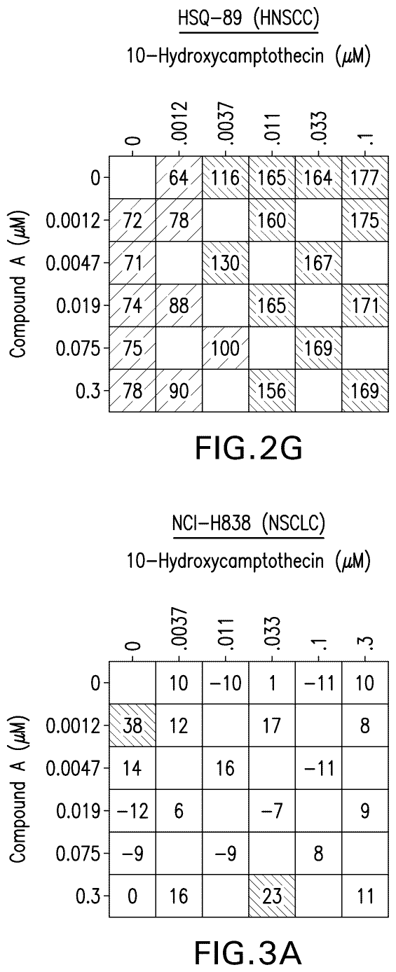

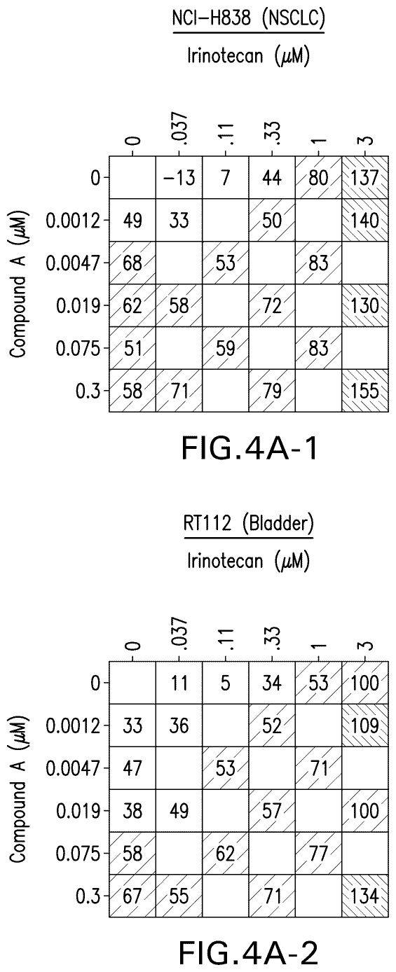

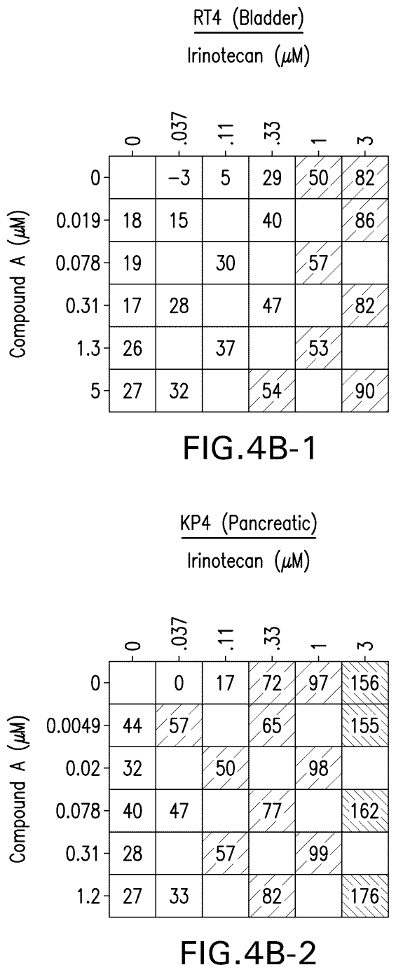

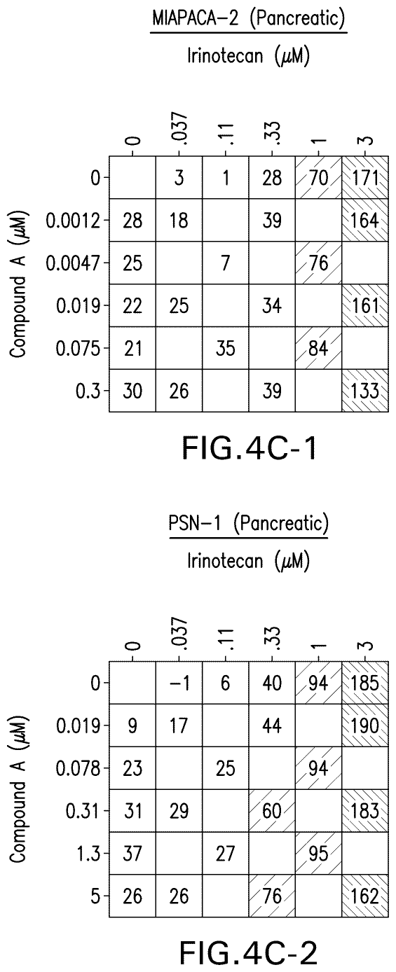

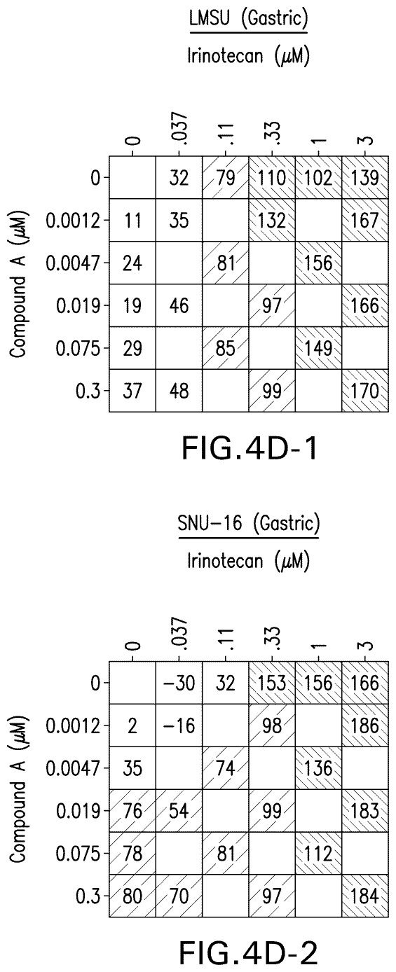

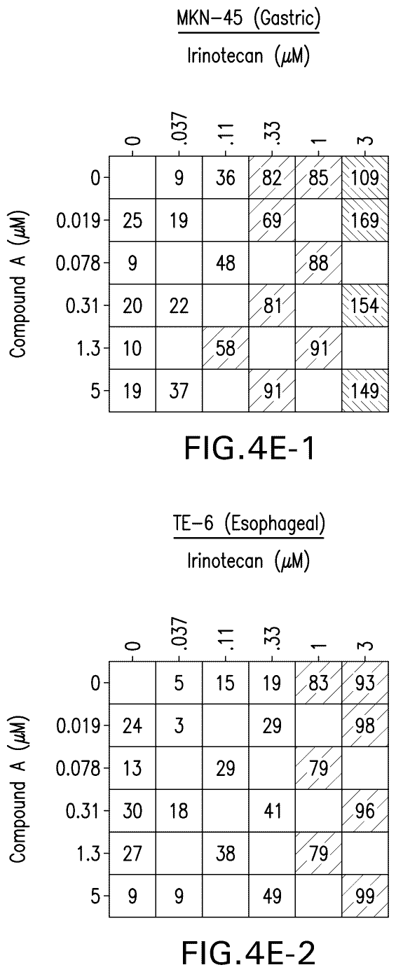

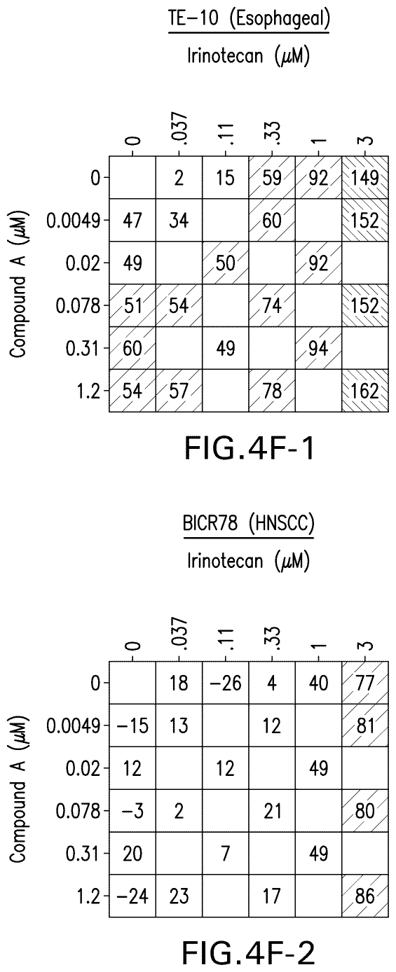

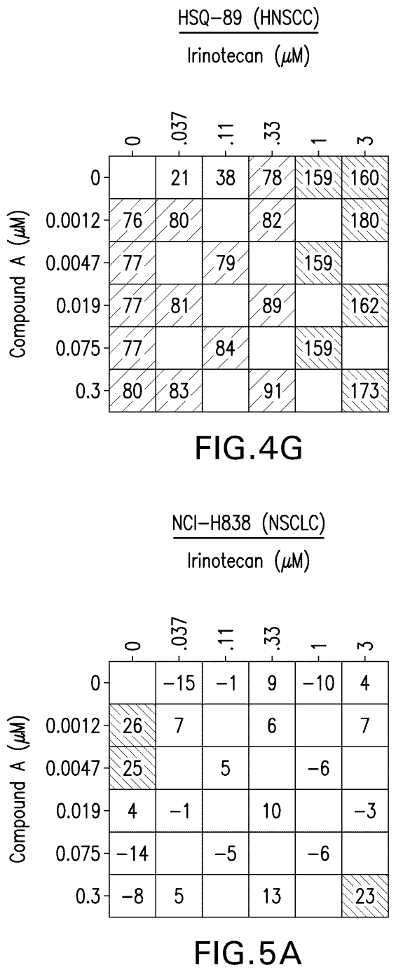

Enhanced growth inhibition for the combination was also observed in 1/2 wild type cell lines at a limited dose range between 39-156 nM of Compound B (Tables 6A-6B). While synergy was calculated by Bliss Independence in this cell line, the potency shift in the dose response curve was not as large as that observed in the MTAP-deficient cell lines. In FIG. 1A-1 to FIG. 1A-5 and FIG. 1B-1 to FIG. 1B-2, growth inhibition is presented as percent of DMSO control for a dose titration of single agent Compound B. Dose response curves were generated using a four-parameter fit for five MTAP-deficient cell lines (FIG. 1A-1 to FIG. 1A-5) and two wild type cell lines (FIGS. 1B-1 and 1B-2).

Tables 5A-5E: Synergistic Growth Inhibition with Compound B in Combination with 1000 nM Compound A Across Five MTAP-deficient Cell Lines

Synergy was measured across the Compound B titration with 1000 nM Compound A by Bliss Independence analysis. Bliss scores >10 are indicated by “*” and are considered synergistic. Bliss scores are valid for combinations exhibiting greater than 20% growth inhibition (GDI <80). Synergy as reported using Bliss may be underrepresented within cytotoxic dose ranges. Growth death index values are shown for the combination titration and each single agent Compound A (Cmpd A) and Compound B (Cmpd B) on a scale of −100 to 100 with the midpoint (0) being the number of cells at the beginning of the combination (T₀). Bliss and GDI values are shown for five MTAP-deficient cell lines: NCI-H838 (5A), SW900 (5B), RT112/84 (5C), UMUC5 (5D), and UMUC11 (5E).

TABLE 5A
NCI-H838

| Compound B Titration + | | | | |
|---|---|---|---|---|
| 1000 nM Compound A Combination | | | Single Agents | |
| Dose (nM) | Bliss Score | GDI | Cmpd. B GDI | Cmpd. A GDI |
| 20000 | 1 | −64 | −1 | |
| 10000 | 21* | 4 | 61 | |
| 5000 | 28* | 8 | 87 | |
| 2500 | 18* | 5 | 58 | |
| 1250 | 14* | 6 | 48 | 52 |
| 625 | 14* | 8 | 55 | |
| 312.5 | 15* | 11 | 65 | |
| 156.3 | 14* | 17 | 74 | |
| 78.1 | 17* | 24 | 100 | |
| 39.1 | 12* | 29 | 100 | |
| 19.5 | 6 | 35 | 100 | |
| 9.8 | −3 | 43 | 100 | |
| 4.9 | −3 | 44 | 100 | |
| 2.4 | −6 | 46 | 100 | |
| 1.2 | −9 | 51 | 100 | |
| 0.6 | −4 | 45 | 100 | |
| 0.3 | −2 | 43 | 100 | |
| 0.2 | 3 | 38 | 100 | |
| 0.1 | −11 | 53 | 100 | |
| 0.04 | −5 | 45 | 100 | |

TABLE 5B
SW900

| Compound B Titration + | | | | |
|---|---|---|---|---|
| 1000 nM Compound A Combination | | | Single Agents | |
| Dose (nM) | Bliss Score | GDI | Cmpd. B GDI | Cmpd. A GDI |
| 20000 | 14* | 12 | 28 | |
| 10000 | 30* | 31 | 64 | |
| 5000 | 29* | 43 | 75 | |
| 2500 | 30* | 50 | 83 | |
| 1250 | 25* | 61 | 89 | 99 |
| 625 | 13* | 71 | 86 | |
| 312.5 | 16 | 82 | 100 | |
| 156.3 | 10 | 88 | 100 | |
| 78.1 | 9 | 88 | 100 | |
| 39.1 | −1 | 97 | 99 | |
| 19.5 | 1 | 97 | 100 | |
| 9.8 | 4 | 94 | 100 | |
| 4.9 | −1 | 99 | 100 | |
| 2.4 | 2 | 95 | 99 | |
| 1.2 | 1 | 96 | 100 | |
| 0.6 | 6 | 92 | 100 | |
| 0.3 | 4 | 94 | 100 | |
| 0.2 | 6 | 92 | 100 | |
| 0.1 | 1 | 98 | 100 | |
| 0.04 | 4 | 94 | 100 | |

TABLE 5C
RT112/84

| Compound B Titration + | | | | |
|---|---|---|---|---|
| 1000 nM Compound A Combination | | | Single Agents | |
| Dose (nM) | Bliss Score | GDI | Cmpd. B GDI | Cmpd. A GDI |
| 20000 | 0 | −91 | −86 | |
| 10000 | 2 | −75 | 2 | |
| 5000 | 19* | 1 | 33 | |
| 2500 | 16* | 3 | 31 | |
| 1250 | 13* | 5 | 31 | 60 |
| 625 | 5 | 9 | 24 | |
| 312.5 | 6 | 12 | 30 | |
| 156.3 | 8 | 30 | 63 | |
| 78.1 | 14* | 46 | 100 | |
| 39.1 | 7 | 53 | 100 | |
| 19.5 | 11* | 49 | 100 | |
| 9.8 | 4 | 56 | 100 | |
| 4.9 | 0 | 59 | 100 | |
| 2.4 | 7 | 52 | 100 | |
| 1.2 | −1 | 61 | 100 | |
| 0.6 | 3 | 57 | 100 | |
| 0.3 | 4 | 55 | 100 | |
| 0.2 | 8 | 52 | 100 | |
| 0.1 | 12* | 48 | 100 | |
| 0.04 | 6 | 55 | 100 | |

TABLE 5D
UMUC5

| Compound B Titration + | | | | |
|---|---|---|---|---|
| 1000 nM Compound A Combination | | | Single Agents | |
| Dose (nM) | Bliss Score | GDI | Cmpd. B GDI | Cmpd. A GDI |
| 20000 | 0 | −89 | −91 | |
| 10000 | 0 | −77 | −87 | |
| 5000 | 3 | −71 | 5 | |
| 2500 | 8 | 1 | 15 | |
| 1250 | 6 | 3 | 16 | 59 |
| 625 | 9 | 1 | 17 | |
| 312.5 | 12* | 3 | 26 | |
| 156.3 | 34* | 9 | 75 | |
| 78.1 | 28* | 30 | 100 | |
| 39.1 | 9 | 50 | 100 | |
| 19.5 | 1 | 57 | 100 | |
| 9.8 | 0 | 59 | 100 | |
| 4.9 | −7 | 65 | 99 | |
| 2.4 | −1 | 59 | 100 | |
| 1.2 | 1 | 55 | 96 | |
| 0.6 | 6 | 53 | 100 | |
| 0.3 | 2 | 56 | 100 | |
| 0.2 | 4 | 55 | 100 | |
| 0.1 | 6 | 52 | 100 | |
| 0.04 | −2 | 58 | 97 | |

TABLE 5E
UMUC11

| Compound B Titration + | | | | |
|---|---|---|---|---|
| 1000 nM Compound A Combination | | | Single Agents | |
| Dose | Bliss | | Cmpd. B | Cmpd. A |
| (nM) | Score | GDI | GDI | GDI |
| 20000 | 0 | −87 | −88 | |
| 10000 | 18* | 11 | 32 | |
| 5000 | 32* | 26 | 63 | |
| 2500 | 32* | 36 | 74 | |
| 1250 | 30* | 34 | 70 | 92 |
| 625 | 27* | 39 | 72 | |
| 312.5 | 38* | 41 | 87 | |
| 156.3 | 33* | 56 | 100 | |
| 78.1 | 24* | 67 | 100 | |
| 39.1 | 14* | 78 | 100 | |
| 19.5 | 9 | 83 | 100 | |
| 9.8 | 6 | 86 | 100 | |
| 4.9 | 8 | 84 | 100 | |
| 2.4 | 6 | 86 | 100 | |
| 1.2 | 6 | 86 | 100 | |
| 0.6 | 2 | 90 | 100 | |
| 0.3 | 13* | 79 | 100 | |
| 0.2 | 11 | 81 | 100 | |
| 0.1 | 9 | 83 | 100 | |
| 0.04 | 2 | 90 | 100 | |

Tables 6A-6B: Synergistic Growth Inhibition with Compound B in Combination with 1000 nM Compound A Across Two Wild Type Cell Lines

Synergy was measured across the Compound B titration with 1000 nM Compound A by Bliss Independence analysis. Bliss scores >10 are indicated by “*” and are considered synergistic. Bliss scores were only calculated for combinations exhibiting greater than 20% growth inhibition. Synergy as reported using Bliss may be underrepresented within cytotoxic dose ranges. Growth death index values are shown for the combination titration and each single agent Compound A (Cmpd A) and Compound B (Cmpd B) on a scale of −100 to 100 with the midpoint (0) being the number of cells at the beginning of the combination (T₀). Bliss and GDI values are shown for five MTAP-deficient cell lines: NCI-1H520 (6A), TCCSUP (6B).

TABLE 6A
NCI-H520

| Compound B Titration + | | | | |
|---|---|---|---|---|
| 1000 nM Compound A Combination | | | Single Agents | |
| Dose | Bliss | | Cmpd. B | Cmpd. A |
| (nM) | Score | GDI | GDI | GDI |
| 20000 | 0 | −56 | −57 | |
| 10000 | 5 | 12 | 17 | |
| 5000 | −12 | 73 | 61 | |
| 2500 | 6 | 86 | 92 | |
| 1250 | −1 | 99 | 92 | 100 |
| 625 | −14 | 95 | 77 | |
| 312.5 | 6 | 79 | 85 | |
| 156.3 | 3 | 99 | 100 | |
| 78.1 | 0 | 100 | 100 | |
| 39.1 | 0 | 100 | 100 | |
| 19.5 | 0 | 100 | 100 | |
| 9.8 | 0 | 100 | 100 | |
| 4.9 | 0 | 100 | 100 | |
| 2.4 | 0 | 100 | 100 | |
| 1.2 | 0 | 100 | 100 | |
| 0.6 | 5 | 100 | 100 | |
| 0.3 | 3 | 100 | 100 | |
| 0.2 | 0 | 100 | 100 | |
| 0.1 | 3 | 100 | 100 | |
| 0.04 | 0 | 100 | 100 | |

TABLE 6B
TCCSUP

| Compound B Titration + | | | | |
|---|---|---|---|---|
| 1000 nM Compound A Combination | | | Single Agents | |
| Dose | Bliss | | Cmpd. B | Cmpd. A |
| (nM) | Score | GDI | GDI | GDI |
| 20000 | 0 | −93 | −95 | |
| 10000 | 0 | −20 | −5 | |
| 5000 | −1 | 6 | 6 | |
| 2500 | 3 | 6 | 11 | |
| 1250 | 3 | 6 | 10 | 88 |
| 625 | 1 | 7 | 8 | |
| 312.5 | 4 | 9 | 15 | |
| 156.3 | 16* | 18 | 38 | |
| 78.1 | 15* | 52 | 76 | |
| 39.1 | 15* | 73 | 100 | |
| 19.5 | 4 | 84 | 100 | |
| 9.8 | 1 | 87 | 100 | |
| 4.9 | 0 | 88 | 100 | |
| 2.4 | 6 | 82 | 100 | |
| 1.2 | 4 | 84 | 100 | |
| 0.6 | 3 | 85 | 100 | |
| 0.3 | 4 | 85 | 100 | |
| 0.2 | 7 | 82 | 100 | |
| 0.1 | 9 | 79 | 100 | |
| 0.04 | 13 | 75 | 100 | |

### Example 3: Proliferation Screen Identifies Combination Benefit with MAT2A Inhibition and Indisulam

### Materials and Methods

A 10-day proliferation screen was performed in 15 pancreatic, non-small cell lung, and bladder cancer cell lines: 10 MTAP null and 5 MTAP wild-type. Optimal cell seeding for all cell lines was determined by assessing the growth over a range of seeding densities in a 384-well format to identify conditions that permitted proliferation for 10 days. Cells were then plated at the optimal seeding density in the presence of 20-150 nM of Compound A or DMSO vehicle control. Cells were incubated at 37° C. with 5% CO₂ for 4 days to allow for target engagement of the pre-treatment compound. Maintaining the pre-treatment condition, cells were also treated with an 11-point, three-fold titration series of compounds from a chemically diverse library of 424 compounds. The combination compound concentrations ranged from 0.2 nM to 14,679 nM. A plate of cells was harvested at the time of combination compound addition to quantify the number of cells at the start of the combination (T₀). For cell quantification, the harvested cells were lysed with CellTiter-Glo (CTG) (Promega) reagent according to the manufacturer's protocol and the chemiluminescent signal was detected on a Synergy Neo plate reader (ThermoFisher, serial #140715A). CTG estimates cell number through detection of cellular ATP levels. Cells were incubated with the drug combinations at 37° C. with 5% CO₂ for an additional 6 days for a total of a 10-day assay including the pre-treatment. Cells were then lysed with CTG and the chemiluminescent signal was measured. CTG values obtained after the 10-day treatment were background subtracted, expressed as a percent of the T₀ value, and plotted against compound concentration. Data were fit with a four-parameter equation to generate concentration response curves. Growth IC50 values and maximal growth inhibition were compared between the DMSO and the Compound A pre-treated cells for each combination titration. Combination hit calling was based on the observation of >2-fold shift in growth IC50 and/or greater than 20% decrease in growth inhibition.

### Results

424 compounds were tested in combination with Compound A in 10 MTAP null and 5 MTAP wild type cell lines. Of these compounds, Indisulam was identified as the most consistent combination hit in the screen with growth IC50 shifts ≥2-fold observed with Compound A pre-treatment in 7/10 MTAP-deficient and 1/5 wild type cell lines (Table 7).

Table 7: Growth IC₅₀ Values with and without Compound A Pre-Treatment in 10 Cancer Cell Lines

Growth IC₅₀ and fold change values are shown for Indisulam and Compound A. Growth C₅₀ fold shifts ≥2-fold (indicated by “*”) were observed in 7/10 MTAP-deficient and 1/5 wild type cell lines. The NCI-H838 cell line was tested at two concentrations of Compound A (2 nM and 358 nM). Fold shifts ≥2-fold in the growth IC₅₀ values were observed for both of these conditions.

Indisulam Growth IC₅₀ Values with DMSO or Compound A Pre-treatment

| | | Cmpd A | Growth | Growth | |
|---|---|---|---|---|---|
| | MTAP | Pre-Treatment | IC₅₀ (nM) | IC₅₀ (nM) | Fold |
| Cell Line | Status | Dose (nM) | DMSO | Cmpd A | Change |
| Panc 03.27 MTAP | Knock-out | 15 | 2171.6 | 873.4 | 2.5* |
| Knock-out Clone 31 | | | | | |
| NCI-H838 | Null | 20 | 1052.3 | 538.8 | 2.0* |
| NCI-H838 | Null | 150 | 806.7 | 341.7 | 2.4* |
| HuP-T4 | Null | 150 | 1072.7 | 549.1 | 2.0* |
| RT112/84 | Null | 150 | 913.3 | 229.1 | 4.0* |
| UMUC5 | Null | 150 | 1829.7 | 807.2 | 2.3* |
| UMUC11 | Null | 150 | 864.4 | 1271.0 | 0.7 |
| SKGT4 | Null | 150 | 1060.8 | 516.2 | 2.1* |
| NCI-H2228 | Null | 80 | 166.9 | 190.8 | 0.9 |
| SW900 | Null | 80 | 2276.3 | 1097.3 | 2.1* |
| NCI-H2170 | Null | 80 | 7167.1 | 5549.8 | 1.3 |
| Panc 03.27 Parent | Wild Type | 15 | 814.0 | 617.8 | 1.3 |
| NCI-H520 | Wild Type | 80 | 727.0 | 790.0 | 0.9 |
| J82 | Wild Type | 150 | 12497.2 | 7104.6 | 1.8 |
| TCCSUP | Wild Type | 150 | 8659.1 | 6077.9 | 1.4 |
| OE33 | Wild Type | 150 | 12058.2 | 5370.7 | 2.2* |

### Example 4: In Vitro Synergistic Growth Inhibition with Equimolar Combinations of a MAT2A Inhibitor and Four Structurally Different Splicing Inhibitor Sulfonamides

### Materials and Methods

A 10-day proliferation assay was performed in a panel of 5 MTAP-deficient and 2 wild type non-small cell lung and bladder cancer cell lines. Optimal cell seeding for all cell lines was determined by assessing the growth over a range of seeding densities in a 384-well format to identify conditions that permitted proliferation for 10 days. Cells were then plated at the optimal seeding density and treated with 20-point, two-fold dilution series of Compound A, the splicing inhibitor sulfonamide (SPLAM), or an equimolar combination of Compound A and the SPLAM. Combinations tested included Compound A in combination with four structurally different SPLAMs. Concentrations tested for Compound A and each SPLAM alone or in combination ranged from 0.04 nM to 20,000 nM. A plate of cells was harvested at the time of compound addition to quantify the number of cells at the start of the combination (T₀). For cell quantification, the harvested cells were lysed with Promega CellTiter-Glo (CTG) reagent according to the manufacturer's protocol and the chemiluminescent signal was detected on a Synergy Neo plate reader. CTG estimates cell number through detection of cellular ATP levels. Cells were incubated with the drug combinations at 37° C. with 5% CO₂ for 10 days. Cells were then lysed with CTG and the chemiluminescent signal was measured.

### Analysis

CTG values obtained after the 10-day treatment were background subtracted and expressed as a percent of the T₀ value. Data were plotted against compound concentration and fit with a four-parameter equation to generate dose response curves for each single agent and the equimolar combination. Growth ICx (gICx) values ranging from gIC30 to gIC100 were interpolated from the fitted curves. Synergistic growth inhibition was assessed by determination of Combination Index (CI) values at these various points across the titration using the mutually non-exclusive equation [Chou, 1983; Chou, 1981] shown below where A equals the gICx value of Compound A, B equals the gICx value of the SPLAM, and A+B or B+A equals the gICx value of the equimolar combination. A Combination Index value less than 1.0 was considered synergistic.

$$CI = \frac{{gIC}_{x} of ( A + B )}{{gIC}_{x} of A} + \frac{{gIC}_{x} of ( B + A )}{{gIC}_{x} of B} + \frac{( {gIC}_{x} of ( A + B ) ) ( {gIC}_{x} of ( B + A ) )}{( {gIC}_{x} of A ) ( {gIC}_{x} of B )}$$

### Results

Synergistic growth inhibition was observed with Compound A in combination with each of four different SPLAMs at multiple gICx points along the combination titration in a panel of MTAP-deficient cell lines (Tables 8A-8E, 9A-9E, 10A-10E, 11A-11E, and 12A-12E). Combination synergy was also observed in two wild type cell lines, especially for the combinations with the more potent SPLAMs Indisulam and E7820 (Tables 8F-8G, 9F-9G, 10F-10G, and 11F-11G). While considered synergistic, the Combination Index values in the wild type cell lines were higher than those observed in the MTAP-deficient cell lines. In comparison to the wild type cell lines, the MTAP-deficient cell lines exhibited greater sensitivity to the combination with greater growth inhibition.

Tables 8A-8G: Synergistic Growth Inhibition with Equimolar Combinations of Compound A and Indisulam Across Five MTAP-deficient and Two Wild Type Cell Lines

Synergy was measured for equimolar combinations of Compound A and Indisulam utilizing the mutually non-exclusive Combination Index equation across gICx values ranging from gIC30-gIC100. Combination Index values are shown for MTAP-deficient cell lines (8A-8E) and wild type lines (8F-8G).

TABLE 8A
NCI-H838

| | Cmpd A | Indisulam | 1:1 Combo | Combination |
|---|---|---|---|---|
| | (nM) | (nM) | (nM) | Index |
| gIC30 | 102 | 276 | 63 | 1.0 |
| gIC40 | 189 | 300 | 75 | 0.8 |
| gIC50 | 594 | 325 | 89 | 0.5 |
| gIC60 | 11309 | 355 | 107 | 0.3 |
| gIC70 | 20000 | 393 | 132 | 0.3 |
| gIC80 | 20000 | 448 | 171 | 0.4 |
| gIC90 | 20000 | 551 | 252 | 0.5 |
| gIC100 | 20000 | 20000 | 984 | 0.1 |

TABLE 8B
SW900

| | Cmpd A | Indisulam | 1:1 Combo | Combination |
|---|---|---|---|---|
| | (nM) | (nM) | (nM) | Index |
| gIC30 | 19994 | 295 | 135 | 0.46 |
| gIC40 | 20000 | 300 | 151 | 0.5 |
| gIC50 | 20000 | 305 | 168 | 0.6 |
| gIC60 | 20000 | 310 | 186 | 0.6 |
| gIC70 | 20000 | 315 | 208 | 0.7 |
| gIC80 | 20000 | 321 | 238 | 0.8 |
| gIC90 | 20000 | 331 | 285 | 0.9 |
| gIC100 | 20000 | 357 | 424 | 1.2 |

TABLE 8C
RT112/84

| | Cmpd A | Indisulam | 1:1 Combo | Combination |
|---|---|---|---|---|
| | (nM) | (nM) | (nM) | Index |
| gIC30 | 345 | 249 | 50 | 0.4 |
| gIC40 | 1111 | 273 | 66 | 0.3 |
| gIC50 | 3405 | 298 | 85 | 0.3 |
| gIC60 | 12951 | 327 | 110 | 0.3 |
| gIC70 | 20000 | 363 | 145 | 0.4 |
| gIC80 | 20000 | 414 | 202 | 0.5 |
| gIC90 | 20000 | 510 | 326 | 0.7 |
| gIC100 | 20000 | 20000 | 1136 | 0.1 |

TABLE 8D
UMUC5

| | Cmpd A | Indisulam | 1:1 Combo | Combination |
|---|---|---|---|---|
| | (nM) | (nM) | (nM) | Index |
| gIC30 | 420 | 310 | 86 | 0.6 |
| gIC40 | 1291 | 363 | 114 | 0.4 |
| gIC50 | 4283 | 420 | 148 | 0.4 |
| gIC60 | 13142 | 487 | 195 | 0.4 |
| gIC70 | 20000 | 574 | 264 | 0.5 |
| gIC80 | 20000 | 707 | 390 | 0.6 |
| gIC90 | 20000 | 990 | 747 | 0.8 |
| gIC100 | 20000 | 20000 | 20000 | 3.0 |

TABLE 8E
UMUC11

| | Cmpd A | Indisulam | 1:1 Combo | Combination |
|---|---|---|---|---|
| | (nM) | (nM) | (nM) | Index |
| gIC30 | 20000 | 369 | 218 | 0.6 |
| gIC40 | 20000 | 417 | 264 | 0.7 |
| gIC50 | 20000 | 472 | 318 | 0.7 |
| gIC60 | 20000 | 539 | 388 | 0.8 |
| gIC70 | 20000 | 628 | 486 | 0.8 |
| gIC80 | 20000 | 769 | 649 | 0.9 |
| gIC90 | 20000 | 1106 | 1035 | 1.0 |
| gIC100 | 20000 | 20000 | 20000 | 3.0 |

TABLE 8F
NCI-H520

| | Cmpd A | Indisulam | 1:1 Combo | Combination |
|---|---|---|---|---|
| | (nM) | (nM) | (nM) | Index |
| gIC30 | 20000 | 470 | 453 | 1.0 |
| gIC40 | 20000 | 546 | 505 | 0.98 |
| gIC50 | 20000 | 633 | 561 | 0.9 |
| gIC60 | 20000 | 743 | 626 | 0.9 |
| gIC70 | 20000 | 897 | 711 | 0.9 |
| gIC80 | 20000 | 1164 | 841 | 0.8 |
| gIC90 | 20000 | 2068 | 1140 | 0.7 |
| gIC100 | 20000 | 20000 | 20000 | 3.0 |

TABLE 8G
TCCSUP

| | Cmpd A | Indisulam | 1:1 Combo | Combination |
|---|---|---|---|---|
| | (nM) | (nM) | (nM) | Index |
| gIC30 | 14461 | 20000 | 1479 | 0.2 |
| gIC40 | 19467 | 20000 | 3847 | 0.4 |
| gIC50 | 20000 | 20000 | 13077 | 1.7 |
| gIC60 | 20000 | 20000 | 20000 | 3.0 |
| gIC70 | 20000 | 20000 | 20000 | 3.0 |
| gIC80 | 20000 | 20000 | 20000 | 3.0 |
| gIC90 | 20000 | 20000 | 20000 | 3.0 |
| gIC100 | 20000 | 20000 | 20000 | 3.0 |

Tables 9A-9G: Synergistic Growth Inhibition with Equimolar Combinations of Compound A and E7820 Across Five MTAP-Deficient and Two Wild Type Cell Lines

Synergy was measured for equimolar combinations of Compound A and E7820 utilizing the mutually non-exclusive Combination Index equation across gICx values ranging from gIC30-gIC100. Combination Index values are shown for MTAP-deficicient cell lines (9A-9E) and wild type lines (9F-9G).

TABLE 9A
NCI-H838

| | Cmpd A | E7820 | 1:1 Combo | Combination |
|---|---|---|---|---|
| | (nM) | (nM) | (nM) | Index |
| gIC30 | 102 | 328 | 58 | 0.9 |
| gIC40 | 189 | 357 | 71 | 0.7 |
| gIC50 | 594 | 388 | 86 | 0.4 |
| gIC60 | 11309 | 425 | 107 | 0.3 |
| gIC70 | 20000 | 472 | 137 | 0.3 |
| gIC80 | 20000 | 539 | 185 | 0.4 |
| gIC90 | 20000 | 666 | 293 | 0.5 |
| gIC100 | 20000 | 20000 | 1200 | 0.1 |

TABLE 9B
SW900

| | Cmpd A | E7820 | 1:1 Combo | Combination |
|---|---|---|---|---|
| | (nM) | (nM) | (nM) | Index |
| gIC30 | 19994 | 284 | 135 | 0.5 |
| gIC40 | 20000 | 290 | 150 | 0.5 |
| gIC50 | 20000 | 296 | 166 | 0.6 |
| gIC60 | 20000 | 303 | 183 | 0.6 |
| gIC70 | 20000 | 309 | 203 | 0.7 |
| gIC80 | 20000 | 318 | 231 | 0.7 |
| gIC90 | 20000 | 330 | 274 | 0.9 |
| gIC100 | 20000 | 362 | 392 | 1.1 |

TABLE 9C
RT112/84

| | Cmpd A | E7820 | 1:1 Combo | Combination |
|---|---|---|---|---|
| | (nM) | (nM) | (nM) | Index |
| gIC30 | 345 | 213 | 43 | 0.4 |
| gIC40 | 1111 | 237 | 55 | 0.3 |
| gIC50 | 3405 | 262 | 69 | 0.3 |
| gIC60 | 12951 | 290 | 87 | 0.3 |
| gIC70 | 20000 | 326 | 113 | 0.4 |
| gIC80 | 20000 | 375 | 153 | 0.4 |
| gIC90 | 20000 | 466 | 239 | 0.5 |
| gIC100 | 20000 | 10429 | 838 | 0.5 |

TABLE 9D
UMUC5

| | Cmpd A | E7820 | 1:1 Combo | Combination |
|---|---|---|---|---|
| | (nM) | (nM) | (nM) | Index |
| gIC30 | 420 | 330 | 88 | 0.5 |
| gIC40 | 1291 | 390 | 118 | 0.4 |
| gIC50 | 4283 | 460 | 158 | 0.4 |
| gIC60 | 13142 | 547 | 212 | 0.4 |
| gIC70 | 20000 | 673 | 297 | 0.5 |
| gIC80 | 20000 | 900 | 460 | 0.5 |
| gIC90 | 20000 | 1896 | 1028 | 0.6 |
| gIC100 | 20000 | 20000 | 20000 | 3.0 |

TABLE 9E
UMUC11

| | Cmpd A | E7820 | 1:1 Combo | Combination |
|---|---|---|---|---|
| | (nM) | (nM) | (nM) | Index |
| gIC30 | 20000 | 361 | 168 | 0.5 |
| gIC40 | 20000 | 404 | 207 | 0.5 |
| gIC50 | 20000 | 452 | 254 | 0.6 |
| gIC60 | 20000 | 511 | 316 | 0.6 |
| gIC70 | 20000 | 589 | 405 | 0.7 |
| gIC80 | 20000 | 713 | 557 | 0.8 |
| gIC90 | 20000 | 1013 | 930 | 1.0 |
| gIC100 | 20000 | 20000 | 20000 | 3.0 |

TABLE 9F
NCI-H520

| | Cmpd A | E7820 | 1:1 Combo | Combination |
|---|---|---|---|---|
| | (nM) | (nM) | (nM) | Index |
| gIC30 | 20000 | 484 | 313 | 0.7 |
| gIC40 | 20000 | 596 | 378 | 0.7 |
| gIC50 | 20000 | 734 | 456 | 0.7 |
| gIC60 | 20000 | 920 | 557 | 0.7 |
| gIC70 | 20000 | 1214 | 703 | 0.6 |
| gIC80 | 20000 | 1842 | 962 | 0.6 |
| gIC90 | 20000 | 11978 | 1779 | 0.4 |
| gIC100 | 20000 | 20000 | 20000 | 3.0 |

TABLE 9G
TCCSUP

| | Cmpd A | E7820 | 1:1 Combo | Combination |
|---|---|---|---|---|
| | (nM) | (nM) | (nM) | Index |
| gIC30 | 14461 | 20000 | 1969 | 0.3 |
| gIC40 | 19467 | 20000 | 5345 | 0.6 |
| gIC50 | 20000 | 20000 | 16625 | 2.4 |
| gIC60 | 20000 | 20000 | 20000 | 3.0 |
| gIC70 | 20000 | 20000 | 20000 | 3.0 |
| gIC80 | 20000 | 20000 | 20000 | 3.0 |
| gIC90 | 20000 | 20000 | 20000 | 3.0 |
| gIC100 | 20000 | 20000 | 20000 | 3.0 |

Tables 10A-10G: Synergistic Growth Inhibition with Equimolar Combinations of Compound A and Chloroquinoxaline Sulfonamide Across Five MTAP-Deficient and Two Wild Type Cell Lines

Synergy was measured for equimolar combinations of Compound A and Chloroquinoxaline Sulfonamide utilizing the mutually non-exclusive Combination Index equation across gICx values ranging from gIC30-gIC100. Combination Index values are shown for MTAP-deficicient cell lines (10A-10E) and wild type lines (10F-10G).

TABLE 10A
NCI-H838

| | Cmpd A | CQS | 1:1 Combo | Combination |
|---|---|---|---|---|
| | (nM) | (nM) | (nM) | Index |
| gIC30 | 102 | 3755 | 146 | 1.6 |
| gIC40 | 189 | 3954 | 232 | 1.5 |
| gIC50 | 594 | 4173 | 365 | 0.8 |
| gIC60 | 11309 | 4433 | 577 | 0.3 |
| gIC70 | 20000 | 4766 | 940 | 0.3 |
| gIC80 | 20000 | 5256 | 1631 | 0.4 |
| gIC90 | 20000 | 6230 | 3211 | 0.8 |
| gIC100 | 20000 | 20000 | 8680 | 1.1 |

TABLE 10B
SW900

| | Cmpd A | CQS | 1:1 Combo | Combination |
|---|---|---|---|---|
| | (nM) | (nM) | (nM) | Index |
| gIC30 | 19994 | 3476 | 1744 | 0.6 |
| gIC40 | 20000 | 3573 | 2047 | 0.7 |
| gIC50 | 20000 | 3670 | 2373 | 0.8 |
| gIC60 | 20000 | 3773 | 2746 | 1.0 |
| gIC70 | 20000 | 3892 | 3206 | 1.1 |
| gIC80 | 20000 | 4042 | 3834 | 1.3 |
| gIC90 | 20000 | 4267 | 4845 | 1.7 |
| gIC100 | 20000 | 12803 | 7316 | 1.4 |

TABLE 10C
RT112/84

| | Cmpd A | CQS | 1:1 Combo | Combination |
|---|---|---|---|---|
| | (nM) | (nM) | (nM) | Index |
| gIC30 | 345 | 7270 | 202 | 0.6 |
| gIC40 | 1111 | 8097 | 383 | 0.4 |
| gIC50 | 3405 | 8969 | 692 | 0.3 |
| gIC60 | 12951 | 9943 | 1222 | 0.2 |
| gIC70 | 20000 | 11106 | 2148 | 0.3 |
| gIC80 | 20000 | 12627 | 3819 | 0.6 |
| gIC90 | 20000 | 14961 | 7041 | 1.0 |
| gIC100 | 20000 | 20000 | 14220 | 1.0 |

TABLE 10D
UMUC5

| | Cmpd A | CQS | 1:1 Combo | Combination |
|---|---|---|---|---|
| | (nM) | (nM) | (nM) | Index |
| gIC30 | 420 | 4296 | 343 | 1.0 |
| gIC40 | 1291 | 4926 | 663 | 0.7 |
| gIC50 | 4283 | 5611 | 1192 | 0.7 |
| gIC60 | 13142 | 6404 | 2058 | 0.6 |
| gIC70 | 20000 | 7398 | 3491 | 0.7 |
| gIC80 | 20000 | 8792 | 5917 | 1.2 |
| gIC90 | 20000 | 11197 | 10210 | 1.9 |
| gIC100 | 20000 | 19275 | 20000 | 3.1 |

TABLE 10E
UMUC11

| | Cmpd A | CQS | 1:1 Combo | Combination |
|---|---|---|---|---|
| | (nM) | (nM) | (nM) | Index |
| gIC30 | 20000 | 8118 | 3988 | 0.8 |
| gIC40 | 20000 | 8930 | 5063 | 0.97 |
| gIC50 | 20000 | 9833 | 6315 | 1.2 |
| gIC60 | 20000 | 10922 | 7830 | 1.4 |
| gIC70 | 20000 | 12392 | 9747 | 1.7 |
| gIC80 | 20000 | 14841 | 12343 | 2.0 |
| gIC90 | 20000 | 19952 | 16335 | 2.3 |
| gIC100 | 20000 | 20000 | 20000 | 3.0 |

TABLE 10F
NCI-H520

| | Cmpd A | CQS | 1:1 Combo | Combination |
|---|---|---|---|---|
| | (nM) | (nM) | (nM) | Index |
| gIC30 | 20000 | 7282 | 5841 | 1.3 |
| gIC40 | 20000 | 8610 | 6688 | 1.4 |
| gIC50 | 20000 | 10125 | 7672 | 1.4 |
| gIC60 | 20000 | 11968 | 8927 | 1.5 |
| gIC70 | 20000 | 14407 | 10778 | 1.7 |
| gIC80 | 20000 | 18134 | 15234 | 2.3 |
| gIC90 | 20000 | 20000 | 20000 | 3.0 |
| gIC100 | 20000 | 20000 | 20000 | 3.0 |

TABLE 10G
TCCSUP

| | Cmpd A | CQS | 1:1 Combo | Combination |
|---|---|---|---|---|
| | (nM) | (nM) | (nM) | Index |
| gIC30 | 14461 | 20000 | 3752 | 0.5 |
| gIC40 | 19467 | 20000 | 8012 | 0.98 |
| gIC50 | 20000 | 20000 | 15250 | 2.1 |
| gIC60 | 20000 | 20000 | 20000 | 3.0 |
| gIC70 | 20000 | 20000 | 20000 | 3.0 |
| gIC80 | 20000 | 20000 | 20000 | 3.0 |
| gIC90 | 20000 | 20000 | 20000 | 3.0 |
| gIC100 | 20000 | 20000 | 20000 | 3.0 |

Tables 11A-11G: Synergistic Growth Inhibition with Equimolar Combinations of Compound A and Tasisulam Across Five MTAP-Deficient and Two Wild Type Cell Lines

Synergy was measured for equimolar combinations of Compound A and Tasisulam utilizing the mutually non-exclusive Combination Index equation across gICx values ranging from gIC30-gIC100. Combination Index values are shown for MTAP-deficicient cell lines (11A-11E) and wild type lines (11F-11G).

TABLE 11A
NCI-H838

| | Cmpd A | Tasisulam | 1:1 Combo | Combination |
|---|---|---|---|---|
| | (nM) | (nM) | (nM) | Index |
| gIC30 | 102 | 7419 | 257 | 2.8 |
| gIC40 | 189 | 4551 | 228 | 3.1 |
| gIC50 | 594 | 4930 | 375 | 1.8 |
| gIC60 | 11309 | 5372 | 625 | 0.5 |
| gIC70 | 20000 | 5923 | 1072 | 0.4 |
| gIC80 | 20000 | 6685 | 1923 | 0.7 |
| gIC90 | 20000 | 7973 | 3709 | 1.2 |
| gIC100 | 20000 | 20000 | 8235 | 2.1 |

TABLE 11B
SW900

| | Cmpd A | Tasisulam | 1:1 Combo | Combination |
|---|---|---|---|---|
| | (nM) | (nM) | (nM) | Index |
| gIC30 | 19994 | 20000 | 6642 | 0.8 |
| gIC40 | 20000 | 20000 | 7707 | 0.9 |
| gIC50 | 20000 | 20000 | 8830 | 1.1 |
| gIC60 | 20000 | 20000 | 10070 | 1.3 |
| gIC70 | 20000 | 20000 | 11504 | 1.5 |
| gIC80 | 20000 | 20000 | 13260 | 1.8 |
| gIC90 | 20000 | 20000 | 15576 | 2.2 |
| gIC100 | 20000 | 20000 | 19020 | 2.8 |

TABLE 11C
RT112/84

| | Cmpd A | Tasisulam | 1:1 Combo | Combination |
|---|---|---|---|---|
| | (nM) | (nM) | (nM) | Index |
| gIC30 | 345 | 20000 | 355 | 1.1 |
| gIC40 | 1111 | 20000 | 726 | 0.7 |
| gIC50 | 3405 | 20000 | 1370 | 0.5 |
| gIC60 | 12951 | 20000 | 2460 | 0.3 |
| gIC70 | 20000 | 20000 | 4284 | 0.5 |
| gIC80 | 20000 | 20000 | 7338 | 0.9 |
| gIC90 | 20000 | 20000 | 12512 | 1.6 |
| gIC100 | 20000 | 20000 | 20000 | 3.0 |

TABLE 11D
UMUC5

| | Cmpd A | Tasisulam | 1:1 Combo | Combination |
|---|---|---|---|---|
| | (nM) | (nM) | (nM) | Index |
| gIC30 | 420 | 16121 | 589 | 1.6 |
| gIC40 | 1291 | 16834 | 1277 | 1.2 |
| gIC50 | 4283 | 17471 | 2448 | 1.1 |
| gIC60 | 13142 | 18053 | 4301 | 0.8 |
| gIC70 | 20000 | 18592 | 7093 | 0.9 |
| gIC80 | 20000 | 19096 | 11142 | 1.5 |
| gIC90 | 20000 | 19571 | 16851 | 2.4 |
| gIC100 | 20000 | 20000 | 20000 | 3.0 |

TABLE 11E
UMUC11

| | Cmpd A | Tasisulam | 1:1 Combo | Combination |
|---|---|---|---|---|
| | (nM) | (nM) | (nM) | Index |
| gIC30 | 20000 | 17770 | 7955 | 1.0 |
| gIC40 | 20000 | 18358 | 9004 | 1.2 |
| gIC50 | 20000 | 18879 | 10147 | 1.3 |
| gIC60 | 20000 | 19349 | 11483 | 1.5 |
| gIC70 | 20000 | 19781 | 13192 | 1.8 |
| gIC80 | 20000 | 20000 | 15711 | 2.2 |
| gIC90 | 20000 | 20000 | 20000 | 3.0 |
| gIC100 | 20000 | 20000 | 20000 | 3.0 |

TABLE 11F
NCI-H520

| | Cmpd A | Tasisulam | 1:1 Combo | Combination |
|---|---|---|---|---|
| | (nM) | (nM) | (nM) | Index |
| gIC30 | 20000 | 9551 | 6118 | 1.1 |
| gIC40 | 20000 | 10354 | 7063 | 1.3 |
| gIC50 | 20000 | 11228 | 8105 | 1.4 |
| gIC60 | 20000 | 12274 | 9334 | 1.6 |
| gIC70 | 20000 | 13716 | 10907 | 1.8 |
| gIC80 | 20000 | 16397 | 13196 | 2.0 |
| gIC90 | 20000 | 11978 | 17758 | 6.0 |
| gIC100 | 20000 | 20000 | 20000 | 3.0 |

TABLE 11G
TCCSUP

| | Cmpd A | Tasisulam | 1:1 Combo | Combination |
|---|---|---|---|---|
| | (nM) | (nM) | (nM) | Index |
| gIC30 | 14461 | 20000 | 6152 | 1.0 |
| gIC40 | 19467 | 20000 | 10922 | 1.4 |
| gIC50 | 20000 | 20000 | 17497 | 2.5 |
| gIC60 | 20000 | 20000 | 20000 | 3.0 |
| gIC70 | 20000 | 20000 | 20000 | 3.0 |
| gIC80 | 20000 | 20000 | 20000 | 3.0 |
| gIC90 | 20000 | 20000 | 20000 | 3.0 |
| gIC100 | 20000 | 20000 | 20000 | 3.0 |

Tables 12A-12E: Synergistic Growth Inhibition with Indisulam in Combination with 1000 nM Compound A Across Five MTAP-Deficient Cell Lines

Synergy was measured across the Indisulam titration with 1000 nM Compound A by Bliss Independence analysis. Bliss scores >10 are indicated by “*” and are considered synergistic. Bliss scores are valid for combinations exhibiting greater than 20% growth inhibition (GDI <80). Synergy as reported using Bliss may be underrepresented within cytotoxic dose ranges. Growth death index values are shown for the combination titration and each single agent Compound A (Cmpd A) and Indisulam on a scale of −100 to 100 with the midpoint (0) being the number of cells at the beginning of the combination (T₀). Scores less than 0 are indicated with “**” and are representative of a cytotoxic response. Bliss and GDI values are shown for five MTAP-deficient cell lines: NCI-1H838 (12A), SW900 (12B), RT112/84 (12C), UMUC5 (121D), and UMUC11 (12E).

TABLE 12A
NCI-H838

| | | | Single Agents | |
|---|---|---|---|---|
| | Indisulam + 1 uM Cmpd A | | Indisulam | Cmpd A |
| Dose (nM) | Excess Over Bliss | GDI | GDI | GDI |
| 20000 | 0 | −80** | −52** | |
| 10000 | 0 | −65** | −50** | |
| 5000 | 0 | −44** | −13** | |
| 2500 | 0 | −33** | 1 | |
| 1250 | 1 | −12** | 2 | 49 |
| 625 | 4 | 0 | 11 | |
| 312.5 | 21* | 4 | 54 | |
| 156.3 | 39* | 9 | 100 | |
| 78.1 | 23* | 25 | 100 | |
| 39.1 | 3 | 46 | 100 | |
| 19.5 | −3 | 52 | 100 | |
| 9.8 | −3 | 52 | 100 | |
| 4.9 | −10 | 59 | 100 | |
| 2.4 | 3 | 45 | 100 | |
| 1.2 | −4 | 53 | 100 | |
| 0.6 | −8 | 56 | 100 | |
| 0.3 | −5 | 54 | 100 | |
| 0.2 | −5 | 53 | 100 | |
| 0.1 | −1 | 50 | 100 | |
| 0.04 | 4 | 45 | 100 | |

TABLE 12B
SW900

| | | | Single Agents | |
|---|---|---|---|---|
| | Indisulam + 1 uM Cmpd A | | Indisulam | Cmpd A |
| Dose (nM) | Excess Over Bliss | GDI | GDI | GDI |
| 20000 | 0 | −65** | −60** | |
| 10000 | 1 | −74** | −58** | |
| 5000 | 0 | −63** | −56** | |
| 2500 | 0 | −57** | −61** | |
| 1250 | 0 | −41** | −33** | 105 |
| 625 | 3 | −22** | −3** | |
| 312.5 | 26* | 7 | 34 | |
| 156.3 | 56* | 40 | 100 | |
| 78.1 | 15* | 84 | 100 | |
| 39.1 | 0 | 100 | 100 | |
| 19.5 | 0 | 99 | 100 | |
| 9.8 | 6 | 93 | 100 | |
| 4.9 | 4 | 96 | 100 | |
| 2.4 | 0 | 100 | 100 | |
| 1.2 | −2 | 100 | 100 | |
| 0.6 | 4 | 93 | 100 | |
| 0.3 | 1 | 100 | 100 | |
| 0.2 | −3 | 97 | 94 | |
| 0.1 | 3 | 97 | 98 | |
| 0.04 | 7 | 91 | 100 | |

TABLE 12C
RT112/84

| | | | Single Agents | |
|---|---|---|---|---|
| | Indisulam + 1 uM Cmpd A | | Indisulam | Cmpd A |
| Dose (nM) | Excess Over Bliss | GDI | GDI | GDI |
| 20000 | 0 | −74** | −92** | |
| 10000 | 0 | −23** | −74** | |
| 5000 | 0 | −18** | −63** | |
| 2500 | 0 | −13** | −76** | |
| 1250 | 0 | −20** | −34** | 62 |
| 625 | 8 | −11** | 12 | |
| 312.5 | 25* | 2 | 44 | |
| 156.3 | 46* | 12 | 99 | |
| 78.1 | 31* | 31 | 100 | |
| 39.1 | 20* | 42 | 100 | |
| 19.5 | 11* | 51 | 100 | |
| 9.8 | 3 | 59 | 100 | |
| 4.9 | 10* | 51 | 100 | |
| 2.4 | 6 | 55 | 100 | |
| 1.2 | 2 | 57 | 95 | |
| 0.6 | 3 | 55 | 96 | |
| 0.3 | 6 | 56 | 100 | |
| 0.2 | 11* | 49 | 100 | |
| 0.1 | 9 | 53 | 100 | |
| 0.04 | 8 | 52 | 98 | |

TABLE 12D
UMUC5

| | | | Single Agents | |
|---|---|---|---|---|
| | Indisulam + 1 uM Cmpd A | | Indisulam | Cmpd A |
| Dose (nM) | Excess Over Bliss | GDI | GDI | GDI |
| 20000 | 0 | 1 | 2 | |
| 10000 | −2 | 3 | 2 | |
| 5000 | −3 | 5 | 3 | |
| 2500 | −3 | 4 | 3 | |
| 1250 | −1 | 3 | 5 | 58 |
| 625 | 5 | 10 | 26 | |
| 312.5 | 20* | 20 | 69 | |
| 156.3 | 19* | 35 | 94 | |
| 78.1 | 10 | 49 | 100 | |
| 39.1 | −1 | 58 | 100 | |
| 19.5 | −5 | 64 | 100 | |
| 9.8 | 1 | 57 | 100 | |
| 4.9 | −10 | 68 | 100 | |
| 2.4 | −6 | 64 | 100 | |
| 1.2 | −4 | 61 | 98 | |
| 0.6 | −5 | 64 | 100 | |
| 0.3 | −1 | 59 | 100 | |
| 0.2 | −5 | 62 | 100 | |
| 0.1 | 1 | 57 | 100 | |
| 0.04 | 3 | 55 | 100 | |

TABLE 12E
UMUC11

| | | | Single Agents | |
|---|---|---|---|---|
| | Indisulam + 1 uM Cmpd A | | Indisulam | Cmpd A |
| Dose (nM) | Excess Over Bliss | GDI | GDI | GDI |
| 20000 | 0 | 1 | 1 | |
| 10000 | 0 | 2 | 2 | |
| 5000 | −1 | 3 | 2 | |
| 2500 | 1 | 4 | 5 | |
| 1250 | 5 | 8 | 13 | 100 |
| 625 | 20* | 14 | 35 | |
| 312.5 | 39* | 34 | 73 | |
| 156.3 | 33* | 66 | 100 | |
| 78.1 | 8 | 91 | 100 | |
| 39.1 | 1 | 99 | 100 | |
| 19.5 | −1 | 100 | 100 | |
| 9.8 | −1 | 100 | 100 | |
| 4.9 | −1 | 100 | 100 | |
| 2.4 | 6 | 97 | 100 | |
| 1.2 | −1 | 99 | 100 | |
| 0.6 | 5 | 94 | 100 | |
| 0.3 | 3 | 96 | 100 | |
| 0.2 | 5 | 96 | 100 | |
| 0.1 | 5 | 94 | 100 | |
| 0.04 | 6 | 94 | 100 | |

### Example 5: In Vitro Synergistic Growth Inhibition with a Four Splicing Inhibitor Sulfonamides in Combination with a Fixed Concentration of a MAT2A Inhibitor

### Materials and Methods

A 10-day proliferation assay was performed in a panel of 5 MTAP-deficient and 2 wild type non-small cell lung and bladder cancer cell lines. Optimal cell seeding for all cell lines was determined by assessing the growth over a range of seeding densities in a 384-well format to identify conditions that permitted proliferation for 10 days. Cells were then plated at the optimal seeding density and treated with 20-point, two-fold dilution series of each of four structurally different SPLAMs in combination with a 1000 nM fixed concentration of Compound A. Single agent titrations for Compound A and each SPLAM were included for comparison. Concentrations tested for each SPLAM alone or in combination ranged from 0.04 nM to 20,000 nM. A plate of cells was harvested at the time of compound addition to quantify the number of cells at the start of the combination (T₀). For cell quantification, the harvested cells were lysed with Promega CellTiter-Glo (CTG) reagent according to the manufacturer's protocol and the chemiluminescent signal was detected as described in Example 1 above. Cells were incubated with the drug combinations at 37° C. with 5% CO₂ for 10 days. Cells were then lysed with CTG and the chemiluminescent signal was measured.

### Analysis

CTG values obtained after the 10-day treatment were background subtracted and expressed as a percent of the T₀ value set at 100%. Data were plotted against compound concentration and fit with a four-parameter equation to generate single agent dose response curves. Percent inhibition was determined for each combination and single agent concentration compared to a DMSO control. In addition, the overall net growth or death of the cells was calculated and reported as a growth death index (GDI) value on a scale of −100 to 100 with the midpoint (0) being the number of cells at the beginning of the combination (T₀).

Synergistic growth inhibition was assessed using the Bliss Independence model for each point of the SPLAM titration in combination with 1000 nM Compound A. The observed growth inhibition at each point was compared to the predicted inhibition based on the additive activity of each single agent dose. Predicted inhibition was calculated using (Ea+Eb)−(Ea*Eb) where E equals the effect (inhibition) of each single agent (a and b). A difference between the observed inhibition percentage and the predicted inhibition percentage greater than 10% was considered synergistic. A Bliss score was only reported for combinations eliciting >20% growth inhibition (GDI value <80). Since Bliss is calculated based on growth inhibition, scores within cytotoxic dose ranges may not accurately reflect synergistic cytoxicity.

### Results

Synergistic growth inhibition was observed with each of four structurally different SPLAMs in combination with 1000 nM Compound A across numerous points of the titration as assessed by Bliss Independence in 7/7 cell lines (Tables 13A-13B, 14A-14E, 15A-15B, 16A-16E, 17A-17B, 18A-18E, and 19A-19B). Within the context of each SPLAM, greater growth inhibition and synergy was observed at lower SPLAM doses within the MTAP-deficient cell lines than in the wild type cell lines. Combination cytotoxicity was observed only within the MTAP-deficient NCI-H838, SW900, and RT112/84 cell lines and not in any wild type cell line.

Tables 13A-13B: Synergistic Growth Inhibition with Indisulam in Combination with 1000 nM Compound A Across Two Wild Type Cell Lines

Synergy was measured across the Indisulam titration with 1000 nM Compound A by Bliss Independence analysis. Bliss scores >10 are indicated by “*” and are considered synergistic. Bliss scores are valid for combinations exhibiting greater than 20% growth inhibition (GDI <80). Synergy as reported using Bliss may be underrepresented within cytotoxic dose ranges. Growth death index values are shown for the combination titration and each single agent Compound A (Cmpd A) and Indisulam on a scale of −100 to 100 with the midpoint (0) being the number of cells at the beginning of the combination (T₀). Scores less than 0 are highlighted black and are representative of a cytotoxic response. Bliss and GDI values are shown for five MTAP-deficient cell lines: NCI-H520 (13A), TCCSUP (13B).

TABLE 13A
NCI-H520

| | | | Single Agents | |
|---|---|---|---|---|
| | Indisulam + 1 uM Cmpd A | | Indisulam | Cmpd A |
| Dose (nM) | Excess Over Bliss | GDI | GDI | GDI |
| 20000 | 1 | 2 | 2 | |
| 10000 | 5 | 1 | 6 | |
| 5000 | 2 | 4 | 6 | |
| 2500 | 6 | 6 | 12 | |
| 1250 | 19* | 6 | 25 | 100 |
| 625 | 23* | 21 | 45 | |
| 312.5 | 34* | 60 | 97 | |
| 156.3 | 10 | 90 | 100 | |
| 78.1 | −1 | 100 | 100 | |
| 39.1 | −1 | 100 | 100 | |
| 19.5 | −5 | 100 | 100 | |
| 9.8 | −1 | 100 | 100 | |
| 4.9 | 1 | 100 | 100 | |
| 2.4 | −1 | 100 | 100 | |
| 1.2 | −1 | 100 | 100 | |
| 0.6 | −1 | 100 | 100 | |
| 0.3 | −1 | 100 | 100 | |
| 0.2 | 5 | 99 | 100 | |
| 0.1 | 0 | 100 | 100 | |
| 0.04 | −6 | 100 | 95 | |

TABLE 13B
TCCSUP

| | | | Single Agents | |
|---|---|---|---|---|
| | Indisulam + 1 uM Cmpd A | | Indisulam | Cmpd A |
| Dose (nM) | Excess Over Bliss | GDI | GDI | GDI |
| 20000 | 18* | 40 | 70 | |
| 10000 | 17* | 54 | 86 | |
| 5000 | 25* | 58 | 100 | |
| 2500 | 16* | 67 | 100 | |
| 1250 | 18* | 63 | 100 | 84 |
| 625 | 12* | 72 | 100 | |
| 312.5 | 8 | 76 | 100 | |
| 156.3 | −1 | 84 | 100 | |
| 78.1 | 0 | 84 | 100 | |
| 39.1 | −7 | 91 | 100 | |
| 19.5 | −1 | 85 | 100 | |
| 9.8 | −1 | 85 | 100 | |
| 4.9 | −1 | 85 | 100 | |
| 2.4 | 0 | 84 | 100 | |
| 1.2 | 3 | 81 | 100 | |
| 0.6 | 5 | 79 | 100 | |
| 0.3 | 1 | 83 | 100 | |
| 0.2 | 1 | 83 | 100 | |
| 0.1 | −2 | 86 | 100 | |
| 0.04 | −1 | 85 | 100 | |

Tables 14A-14E: Synergistic Growth Inhibition with E7820 in Combination with 1000 nM Compound A Across Five MTAP-deficient Cell Lines

Synergy was measured across the E7820 titration with 1000 nM Compound A by Bliss Independence analysis. Bliss scores >10 are indicated by “*” and are considered synergistic. Bliss scores are valid for combinations exhibiting greater than 20% growth inhibition (GDI <80). Synergy as reported using Bliss may be underrepresented within cytotoxic dose ranges. Growth death index values are shown for the combination titration and each single agent Compound A (Cmpd A) and Indisulam on a scale of −100 to 100 with the midpoint (0) being the number of cells at the beginning of the combination (T₀). Scores less than 0 are indicated by “**” and are representative of a cytotoxic response. Bliss and GDI values are shown for five MTAP-deficient cell lines: NCI-1H838 (14A), SW900 (14B), RT112/84 (14C), UMUC5 (141)), and UMUC11 (14E).

TABLE 14A
NCI-H838

| | | | Single Agents | |
|---|---|---|---|---|
| | E7820 + 1 uM Cmpd A | | E7820 | Cmpd A |
| Dose (nM) | Excess Over Bliss | GDI | GDI | GDI |
| 20000 | 0 | −66** | −46** | |
| 10000 | 0 | −73** | −64** | |
| 5000 | 0 | −52** | −13** | |
| 2500 | 1 | −61** | 1 | |
| 1250 | 1 | −4** | 4 | 49 |
| 625 | 5 | 1 | 13 | |
| 312.5 | 32* | 5 | 76 | |
| 156.3 | 34* | 15 | 100 | |
| 78.1 | 9 | 39 | 100 | |
| 39.1 | 6 | 42 | 100 | |
| 19.5 | −7 | 56 | 100 | |
| 9.8 | −12 | 61 | 100 | |
| 4.9 | −9 | 57 | 100 | |
| 2.4 | −4 | 53 | 100 | |
| 1.2 | 0 | 49 | 100 | |
| 0.6 | −8 | 57 | 100 | |
| 0.3 | 1 | 48 | 100 | |
| 0.2 | −6 | 54 | 100 | |
| 0.1 | −4 | 52 | 100 | |
| 0.04 | 0 | 49 | 100 | |

TABLE 14B
SW900

| | | | Single Agents | |
|---|---|---|---|---|
| | E7820 + 1 uM Cmpd A | | E7820 | Cmpd A |
| Dose (nM) | Excess Over Bliss | GDI | GDI | GDI |
| 20000 | 1 | −79** | −59** | |
| 10000 | 0 | −68** | −70** | |
| 5000 | 2 | −77** | −47** | |
| 2500 | 1 | −67** | −56** | |
| 1250 | 1 | −58** | −37** | 100 |
| 625 | 3 | −25** | −7** | |
| 312.5 | 21* | 6 | 28 | |
| 156.3 | 57* | 39 | 100 | |
| 78.1 | 15* | 83 | 100 | |
| 39.1 | 3 | 97 | 100 | |
| 19.5 | 0 | 100 | 100 | |
| 9.8 | 2 | 97 | 100 | |
| 4.9 | 0 | 100 | 100 | |
| 2.4 | 4 | 95 | 100 | |
| 1.2 | 2 | 100 | 100 | |
| 0.6 | 2 | 97 | 100 | |
| 0.3 | 0 | 99 | 100 | |
| 0.2 | 6 | 92 | 99 | |
| 0.1 | 3 | 97 | 100 | |
| 0.04 | 2 | 92 | 97 | |

TABLE 14C
RT112/84

| | | | Single Agents | |
|---|---|---|---|---|
| | E7820 + 1 uM Cmpd A | | E7820 | Cmpd A |
| Dose (nM) | Excess Over Bliss | GDI | GDI | GDI |
| 20000 | 0 | −91** | −100** | |
| 10000 | 0 | −80** | −100** | |
| 5000 | 0 | −72** | −100** | |
| 2500 | 0 | −54** | −84** | |
| 1250 | 0 | −64** | −35** | 62 |
| 625 | 4 | −48** | 6 | |
| 312.5 | 18* | 1 | 31 | |
| 156.3 | 44* | 12 | 90 | |
| 78.1 | 29* | 32 | 100 | |
| 39.1 | 12* | 49 | 100 | |
| 19.5 | 6 | 56 | 100 | |
| 9.8 | 0 | 62 | 100 | |
| 4.9 | −2 | 60 | 94 | |
| 2.4 | 10 | 52 | 100 | |
| 1.2 | 13* | 49 | 100 | |
| 0.6 | 3 | 58 | 100 | |
| 0.3 | 1 | 61 | 100 | |
| 0.2 | 13* | 49 | 100 | |
| 0.1 | 5 | 53 | 94 | |
| 0.04 | −2 | 56 | 89 | |

TABLE 14D
UMUC5

Single Agents

| | | |
|---|---|---|
| E7820 + 1 uM Cmpd A | E7820 | Cmpd A |

| Dose (nM) | Excess Over Bliss | GDI | GDI | GDI |
|---|---|---|---|---|
| 20000 | −2 | 4 | 3 | |
| 10000 | −2 | 5 | 6 | |
| 5000 | −2 | 6 | 7 | |
| 2500 | 2 | 6 | 15 | |
| 1250 | −1 | 10 | 15 | 58 |
| 625 | 4 | 15 | 31 | |
| 312.5 | 16* | 27 | 73 | |
| 156.3 | 24* | 33 | 99 | |
| 78.1 | 8 | 50 | 100 | |
| 39.1 | 6 | 52 | 100 | |
| 19.5 | −2 | 60 | 100 | |
| 9.8 | −5 | 63 | 100 | |
| 4.9 | −5 | 63 | 100 | |
| 2.4 | −6 | 64 | 100 | |
| 1.2 | 2 | 56 | 100 | |
| 0.6 | 0 | 58 | 100 | |
| 0.3 | 1 | 57 | 100 | |
| 0.2 | 8 | 50 | 100 | |
| 0.1 | −2 | 60 | 100 | |
| 0.04 | −4 | 63 | 100 | |

TABLE 14E
UMUC11

Single Agents

| | | |
|---|---|---|
| E7820 + 1 uM Cmpd A | E7820 | Cmpd A |

| Dose (nM) | Excess Over Bliss | GDI | GDI | GDI |
|---|---|---|---|---|
| 20000 | 0 | 1 | 1 | |
| 10000 | 0 | 2 | 3 | |
| 5000 | 1 | 3 | 4 | |
| 2500 | 0 | 4 | 5 | |
| 1250 | 2 | 9 | 11 | 100 |
| 625 | 16* | 14 | 31 | |
| 312.5 | 36* | 38 | 76 | |
| 156.3 | 36* | 63 | 100 | |
| 78.1 | 1 | 97 | 100 | |
| 39.1 | −1 | 100 | 100 | |
| 19.5 | 2 | 99 | 100 | |
| 9.8 | 2 | 98 | 100 | |
| 4.9 | 0 | 100 | 100 | |
| 2.4 | 0 | 100 | 100 | |
| 1.2 | 3 | 100 | 100 | |
| 0.6 | 3 | 96 | 100 | |
| 0.3 | −1 | 100 | 100 | |
| 0.2 | 2 | 97 | 100 | |
| 0.1 | 4 | 95 | 100 | |
| 0.04 | 5 | 93 | 100 | |

Tables 15A-15B: Synergistic Growth Inhibition with E7820 in Combination with 1000 nM Compound A Across Two Wild Type Cell Lines

Synergy was measured across the E7820 titration with 1000 nM Compound A by Bliss Independence analysis. Bliss scores >10 are indicated by “*” and are considered synergistic. Bliss scores are valid for combinations exhibiting greater than 20% growth inhibition (GDI <80). Synergy as reported using Bliss may be underrepresented within cytotoxic dose ranges. Growth death index values are shown for the combination titration and each single agent Compound A (Cmpd A) and Indisulam on a scale of −100 to 100 with the midpoint (0) being the number of cells at the beginning of the combination (T₀). Scores less than 0 are indicated by “**” and are representative of a cytotoxic response. Bliss and GDI values are shown for five MTAP-deficient cell lines: NCI-H520 (15A), TCCSUP (15B).

TABLE 15A
NCI-H520

Single Agents

| | | |
|---|---|---|
| E7820 + 1 uM Cmpd A | E7820 | Cmpd A |

| Dose (nM) | Excess Over Bliss | GDI | GDI | GDI |
|---|---|---|---|---|
| 20000 | 2 | 4 | 5 | |
| 10000 | 2 | 6 | 8 | |
| 5000 | 7 | 4 | 11 | |
| 2500 | 16* | 5 | 21 | |
| 1250 | 12* | 20 | 32 | 100 |
| 625 | 32* | 21 | 54 | |
| 312.5 | 23* | 58 | 82 | |
| 156.3 | 11* | 89 | 100 | |
| 78.1 | −1 | 100 | 100 | |
| 39.1 | −1 | 100 | 100 | |
| 19.5 | −1 | 100 | 100 | |
| 9.8 | −1 | 100 | 100 | |
| 4.9 | 0 | 100 | 100 | |
| 2.4 | −1 | 100 | 100 | |
| 1.2 | −1 | 100 | 100 | |
| 0.6 | −1 | 100 | 100 | |
| 0.3 | −1 | 100 | 100 | |
| 0.2 | −1 | 100 | 100 | |
| 0.1 | −1 | 100 | 100 | |
| 0.04 | −1 | 100 | 100 | |

TABLE 15B
TCCSUP

Single Agents

| | | |
|---|---|---|
| E7820 + 1 uM Cmpd A | E7820 | Cmpd A |

| Dose (nM) | Excess Over Bliss | GDI | GDI | GDI |
|---|---|---|---|---|
| 20000 | 23* | 41 | 78 | |
| 10000 | 14* | 62 | 91 | |
| 5000 | 12* | 65 | 92 | |
| 2500 | 16* | 67 | 100 | |
| 1250 | 9 | 75 | 100 | 84 |
| 625 | 14* | 70 | 100 | |
| 312.5 | 9 | 75 | 100 | |
| 156.3 | 6 | 78 | 100 | |
| 78.1 | 2 | 82 | 100 | |
| 39.1 | 3 | 81 | 100 | |
| 19.5 | 5 | 79 | 100 | |
| 9.8 | −5 | 89 | 100 | |
| 4.9 | −8 | 93 | 100 | |
| 2.4 | 0 | 84 | 100 | |
| 1.2 | −2 | 86 | 100 | |
| 0.6 | −2 | 86 | 100 | |
| 0.3 | 4 | 80 | 100 | |
| 0.2 | 7 | 77 | 100 | |
| 0.1 | −2 | 86 | 100 | |
| 0.04 | −4 | 89 | 100 | |

Tables 16A-16E: Synergistic Growth Inhibition with Chloroquinoxaline Sulfonamide in Combination with 1000 nM Compound A Across Five MTAP-deficient Cell Lines

Synergy was measured across the Chloroquinoxaline Sulfonamide titration with 1000 nM Compound A by Bliss Independence analysis. Bliss scores >10 are indicated by and are considered synergistic. Bliss scores are valid for combinations exhibiting greater than 20% growth inhibition (GDI <80). Synergy as reported using Bliss may be underrepresented within cytotoxic dose ranges. Growth death index values are shown for the combination titration and each single agent Compound A (Cmpd A) and Indisulam on a scale of −100 to 100 with the midpoint (0) being the number of cells at the beginning of the combination (T₀). Scores less than 0 are indicated by “**” and are representative of a cytotoxic response. Bliss and GDI values are shown for five MTAP-deficient cell lines: NCI-H838 (16A), SW900 (16B), RT112/84 (16C), UMUC5 (16D), and UMUC11 (16E).

TABLE 16A
NCI-H838

Single Agents

| | | |
|---|---|---|
| CQS + 1 uM Cmpd A | CQS | Cmpd A |

| Dose (nM) | Excess Over Bliss | GDI | GDI | GDI |
|---|---|---|---|---|
| 20000 | 0 | −58** | 0 | |
| 10000 | 1 | −19** | 3 | |
| 5000 | 11* | 2 | 28 | |
| 2500 | 36* | 7 | 100 | |
| 1250 | 21* | 28 | 100 | 49 |
| 625 | 7 | 42 | 100 | |
| 312.5 | −8 | 57 | 100 | |
| 156.3 | −4 | 52 | 100 | |
| 78.1 | −7 | 56 | 100 | |
| 39.1 | −3 | 51 | 100 | |
| 19.5 | −11 | 60 | 100 | |
| 9.8 | 5 | 43 | 100 | |
| 4.9 | −2 | 51 | 100 | |
| 2.4 | −3 | 51 | 100 | |
| 1.2 | −2 | 51 | 100 | |
| 0.6 | −5 | 54 | 100 | |
| 0.3 | −10 | 59 | 100 | |
| 0.2 | −9 | 58 | 100 | |
| 0.1 | −4 | 52 | 100 | |
| 0.04 | −7 | 55 | 100 | |

TABLE 16B
SW900

| | | | Single Agents | |
|---|---|---|---|---|
| | CQS + 1 uM Cmpd A | | CQS | Cmpd A |
| Dose (nM) | Excess Over Bliss | GDI | GDI | GDI |
| 20000 | 0 | −75** | −70** | |
| 10000 | −1 | −38** | −47** | |
| 5000 | 7 | 8 | 15 | |
| 2500 | 53* | 40 | 100 | |
| 1250 | 9 | 90 | 100 | 100 |
| 625 | 1 | 98 | 100 | |
| 312.5 | 0 | 100 | 100 | |
| 156.3 | 0 | 100 | 100 | |
| 78.1 | 0 | 100 | 100 | |
| 39.1 | 0 | 100 | 100 | |
| 19.5 | 0 | 100 | 100 | |
| 9.8 | 5 | 96 | 100 | |
| 4.9 | 0 | 100 | 100 | |
| 2.4 | 2 | 98 | 100 | |
| 1.2 | 1 | 99 | 100 | |
| 0.6 | 0 | 100 | 100 | |
| 0.3 | 1 | 100 | 100 | |
| 0.2 | 3 | 97 | 100 | |
| 0.1 | −21 | 100 | 87 | |
| 0.04 | −1 | 100 | 100 | |

TABLE 16C
RT112/84

| | | | Single Agents | |
|---|---|---|---|---|
| | CQS + 1 uM Cmpd A | | CQS | Cmpd A |
| Dose (nM) | Excess Over Bliss | GDI | GDI | GDI |
| 20000 | 0 | −100** | −39** | |
| 10000 | 24* | −51** | 41 | |
| 5000 | 49* | 6 | 88 | |
| 2500 | 32* | 30 | 100 | |
| 1250 | 17* | 45 | 100 | 62 |
| 625 | 5 | 57 | 100 | |
| 312.5 | 6 | 57 | 100 | |
| 156.3 | 5 | 57 | 100 | |
| 78.1 | 5 | 57 | 100 | |
| 39.1 | 7 | 55 | 100 | |
| 19.5 | 1 | 61 | 100 | |
| 9.8 | 2 | 60 | 100 | |
| 4.9 | 2 | 60 | 100 | |
| 2.4 | 3 | 59 | 100 | |
| 1.2 | 1 | 60 | 100 | |
| 0.6 | 1 | 61 | 100 | |
| 0.3 | 3 | 59 | 100 | |
| 0.2 | −4 | 62 | 94 | |
| 0.1 | 0 | 62 | 100 | |
| 0.04 | 1 | 61 | 100 | |

TABLE 16D
UMUC5

| | | | Single Agents | |
|---|---|---|---|---|
| | CQS + 1 uM Cmpd A | | | Cmpd A |
| Dose (nM) | Excess Over Bliss | GDI | CQS GDI | GDI |
| 20000 | −2 | 2 | 0 | |
| 10000 | 3 | 5 | 13 | |
| 5000 | 15* | 20 | 60 | |
| 2500 | 10 | 45 | 95 | |
| 1250 | 2 | 56 | 100 | 58 |
| 625 | 0 | 58 | 100 | |
| 312.5 | −5 | 64 | 100 | |
| 156.3 | −3 | 61 | 100 | |
| 78.1 | 0 | 58 | 100 | |
| 39.1 | −4 | 62 | 100 | |
| 19.5 | −5 | 63 | 100 | |
| 9.8 | −7 | 65 | 100 | |
| 4.9 | −7 | 65 | 100 | |
| 2.4 | −3 | 61 | 100 | |
| 1.2 | −4 | 62 | 100 | |
| 0.6 | −10 | 68 | 100 | |
| 0.3 | −3 | 61 | 100 | |
| 0.2 | 1 | 57 | 100 | |
| 0.1 | −1 | 59 | 100 | |
| 0.04 | −3 | 62 | 100 | |

TABLE 16E
UMUC11

Single Agents

| | | |
|---|---|---|
| CQS + 1 uM Cmpd A | CQS | Cmpd A |

| Dose (nM) | Excess Over Bliss | GDI | GDI | GDI |
|---|---|---|---|---|
| 20000 | 4 | 7 | 11 | |
| 10000 | 24* | 24 | 49 | |
| 5000 | 39* | 60 | 100 | |
| 2500 | 4 | 95 | 100 | |
| 1250 | −1 | 100 | 100 | 100 |
| 625 | −1 | 100 | 100 | |
| 312.5 | −1 | 100 | 100 | |
| 156.3 | −1 | 100 | 100 | |
| 78.1 | −1 | 100 | 100 | |
| 39.1 | −1 | 100 | 100 | |
| 19.5 | −1 | 100 | 100 | |
| 9.8 | 0 | 100 | 100 | |
| 4.9 | −1 | 100 | 100 | |
| 2.4 | 1 | 98 | 100 | |
| 1.2 | −1 | 100 | 100 | |
| 0.6 | −1 | 100 | 100 | |
| 0.3 | 1 | 98 | 100 | |
| 0.2 | −1 | 100 | 100 | |
| 0.1 | 0 | 100 | 100 | |
| 0.04 | −1 | 100 | 100 | |

Tables 17A-17B: Synergistic Growth Inhibition with Chloroquinoxaline Sulfonamide in Combination with 1000 nM Compound A Across Two Wild Type Cell Lines

Synergy was measured across the Chloroquinoxaline Sulfonamide titration with 1000 nM Compound A by Bliss Independence analysis. Bliss scores >10 are indicated by and are considered synergistic. Bliss scores are valid for combinations exhibiting greater than 20% growth inhibition (GDI <80). Synergy as reported using Bliss may be underrepresented within cytotoxic dose ranges. Growth death index values are shown for the combination titration and each single agent Compound A (Cmpd A) and Indisulam on a scale of −100 to 100 with the midpoint (0) being the number of cells at the beginning of the combination (T₀). Scores less than 0 are indicated by “**” and are representative of a cytotoxic response. Bliss and GDI values are shown for five MTAP-deficient cell lines: NCI-H520 (17A), TCCSUP (17B).

TABLE 17A
NCI-H520

Single Agents

| | | |
|---|---|---|
| CQS + 1 uM Cmpd A | CQS | Cmpd A |

| Dose (nM) | Excess Over Bliss | GDI | GDI | GDI |
|---|---|---|---|---|
| 20000 | 5 | 11 | 16 | |
| 10000 | 25* | 30 | 55 | |
| 5000 | −7 | 88 | 81 | |
| 2500 | 5 | 100 | 100 | |
| 1250 | −1 | 100 | 100 | 100 |
| 625 | −1 | 100 | 100 | |
| 312.5 | −1 | 100 | 100 | |
| 156.3 | −1 | 100 | 100 | |
| 78.1 | −1 | 100 | 100 | |
| 39.1 | −1 | 100 | 100 | |
| 19.5 | −2 | 100 | 100 | |
| 9.8 | −1 | 100 | 100 | |
| 4.9 | −1 | 100 | 100 | |
| 2.4 | −1 | 100 | 100 | |
| 1.2 | −4 | 100 | 100 | |
| 0.6 | −1 | 100 | 100 | |
| 0.3 | −1 | 100 | 100 | |
| 0.2 | −1 | 100 | 100 | |
| 0.1 | −1 | 100 | 100 | |
| 0.04 | 0 | 100 | 100 | |

TABLE 17B
TCCSUP

| | | | Single Agents | |
|---|---|---|---|---|
| | CQS + 1 uM Cmpd A | | | Cmpd A |
| Dose (nM) | Excess Over Bliss | GDI | CQS GDI | GDI |
| 20000 | 28* | 46 | 88 | |
| 10000 | 18* | 62 | 96 | |
| 5000 | 9 | 75 | 100 | |
| 2500 | 7 | 77 | 100 | |
| 1250 | −8 | 92 | 100 | 84 |
| 625 | 0 | 84 | 100 | |
| 312.5 | −4 | 88 | 100 | |
| 156.3 | −5 | 89 | 100 | |
| 78.1 | −11 | 95 | 100 | |
| 39.1 | −7 | 91 | 100 | |
| 19.5 | −7 | 91 | 100 | |
| 9.8 | −10 | 96 | 100 | |
| 4.9 | −10 | 95 | 100 | |
| 2.4 | −1 | 85 | 100 | |
| 1.2 | −8 | 91 | 100 | |
| 0.6 | −8 | 92 | 100 | |
| 0.3 | −10 | 94 | 100 | |
| 0.2 | −1 | 85 | 100 | |
| 0.1 | −6 | 91 | 100 | |
| 0.04 | −3 | 85 | 100 | |

Tables 18A-18E: Synergistic Growth Inhibition with Tasisulam in Combination with 1000 nM Compound A Across Five MTAP-deficient Cell Lines

Synergy was measured across the Tasisulam titration with 1000 nM Compound A by Bliss Independence analysis. Bliss scores >10 are indicated by “*” and are considered synergistic. Bliss scores are valid for combinations exhibiting greater than 20% growth inhibition (GDI <80). Synergy as reported using Bliss may be underrepresented within cytotoxic dose ranges. Growth death index values are shown for the combination titration and each single agent Compound A (Cmpd A) and Indisulam on a scale of −100 to 100 with the midpoint (0) being the number of cells at the beginning of the combination (T₀). Scores less than 0 are indicated by “**” and are representative of a cytotoxic response. Bliss and GDI values are shown for five MTAP-deficient cell lines: NCI-H838 (18A), SW900 (18B), RT112/84 (18C), UMUC5 (18D), and UMUC11 (18E).

TABLE 18A
NCI-H838

| | | | Single Agents | |
|---|---|---|---|---|
| | Tasisulam + 1 uM Cmpd A | | Tasisulam | Cmpd A |
| Dose (nM) | Excess Over Bliss | GDI | GDI | GDI |
| 20000 | 0 | 0** | −2** | |
| 10000 | 14* | 3 | 35 | |
| 5000 | 36* | 13 | 100 | |
| 2500 | 16* | 33 | 100 | |
| 1250 | 10* | 39 | 100 | 49 |
| 625 | −4 | 52 | 100 | |
| 312.5 | −8 | 56 | 100 | |
| 156.3 | 2 | 46 | 100 | |
| 78.1 | −8 | 56 | 100 | |
| 39.1 | −1 | 50 | 100 | |
| 19.5 | 3 | 46 | 100 | |
| 9.8 | 1 | 48 | 100 | |
| 4.9 | −3 | 52 | 100 | |
| 2.4 | 5 | 44 | 100 | |
| 1.2 | −5 | 53 | 100 | |
| 0.6 | 4 | 44 | 100 | |
| 0.3 | 0 | 48 | 100 | |
| 0.2 | 3 | 46 | 100 | |
| 0.1 | 2 | 47 | 100 | |
| 0.04 | 3 | 46 | 100 | |

TABLE 18B
SW900

| | | | Single Agents | |
|---|---|---|---|---|
| | Tasisulam + 1 uM Cmpd A | | Tasisulam | Cmpd A |
| Dose (nM) | Excess Over Bliss | GDI | GDI | GDI |
| 20000 | 11* | 10 | 22 | |
| 10000 | 51* | 46 | 100 | |
| 5000 | 11* | 87 | 100 | |
| 2500 | 0 | 100 | 100 | |
| 1250 | 0 | 100 | 100 | 100 |
| 625 | 1 | 100 | 100 | |
| 312.5 | 2 | 99 | 100 | |
| 156.3 | 3 | 96 | 100 | |
| 78.1 | 1 | 100 | 100 | |
| 39.1 | 4 | 96 | 100 | |
| 19.5 | 2 | 100 | 100 | |
| 9.8 | 3 | 97 | 100 | |
| 4.9 | −1 | 100 | 100 | |
| 2.4 | 4 | 95 | 100 | |
| 1.2 | 7 | 91 | 100 | |
| 0.6 | 0 | 100 | 100 | |
| 0.3 | 0 | 100 | 100 | |
| 0.2 | −1 | 100 | 100 | |
| 0.1 | 1 | 98 | 100 | |
| 0.04 | 10 | 89 | 100 | |

TABLE 18C
RT112/84

| | | | Single Agents | |
|---|---|---|---|---|
| | Tasisulam + 1 uM Cmpd A | | Tasisulam | Cmpd A |
| Dose (nM) | Excess Over Bliss | GDI | GDI | GDI |
| 20000 | 7 | −27** | 14 | |
| 10000 | 50* | 11 | 100 | |
| 5000 | 33* | 29 | 100 | |
| 2500 | 19* | 43 | 100 | |
| 1250 | 3 | 59 | 100 | 62 |
| 625 | 5 | 57 | 100 | |
| 312.5 | −3 | 65 | 100 | |
| 156.3 | 8 | 54 | 100 | |
| 78.1 | 7 | 55 | 100 | |
| 39.1 | 4 | 58 | 100 | |
| 19.5 | 5 | 57 | 100 | |
| 9.8 | 14* | 48 | 100 | |
| 4.9 | 11* | 50 | 100 | |
| 2.4 | 7 | 52 | 99 | |
| 1.2 | 6 | 56 | 100 | |
| 0.6 | 3 | 59 | 100 | |
| 0.3 | 7 | 55 | 100 | |
| 0.2 | 9 | 53 | 100 | |
| 0.1 | 5 | 57 | 100 | |
| 0.04 | 5 | 55 | 100 | |

TABLE 18D
UMUC5

| | | | Single Agents | |
|---|---|---|---|---|
| | Tasisulam + 1 uM Cmpd A | | Tasisulam | Cmpd A |
| Dose (nM) | Excess Over Bliss | GDI | GDI | GDI |
| 20000 | −1 | 1 | 0 | |
| 10000 | 29* | 21 | 86 | |
| 5000 | 12* | 45 | 100 | |
| 2500 | 6 | 52 | 100 | |
| 1250 | 2 | 56 | 100 | 58 |
| 625 | −1 | 59 | 100 | |
| 312.5 | 1 | 57 | 100 | |
| 156.3 | 3 | 55 | 100 | |
| 78.1 | 1 | 57 | 100 | |
| 39.1 | 3 | 55 | 100 | |
| 19.5 | −4 | 63 | 100 | |
| 9.8 | 1 | 57 | 100 | |
| 4.9 | 1 | 57 | 100 | |
| 2.4 | 0 | 58 | 100 | |
| 1.2 | −8 | 66 | 100 | |
| 0.6 | 1 | 58 | 100 | |
| 0.3 | 2 | 56 | 100 | |
| 0.2 | 0 | 59 | 100 | |
| 0.1 | 1 | 57 | 100 | |
| 0.04 | 8 | 50 | 100 | |

TABLE 18E
UMUC11

| | | | Single Agents | |
|---|---|---|---|---|
| | Tasisulam + 1 uM Cmpd A | | Tasisulam | Cmpd A |
| Dose (nM) | Excess Over Bliss | GDI | GDI | GDI |
| 20000 | 10* | 14 | 25 | |
| 10000 | 45* | 52 | 100 | |
| 5000 | 11* | 88 | 100 | |
| 2500 | 2 | 97 | 100 | |
| 1250 | −1 | 100 | 100 | 100 |
| 625 | −1 | 100 | 100 | |
| 312.5 | 0 | 100 | 100 | |
| 156.3 | 7 | 93 | 100 | |
| 78.1 | 1 | 96 | 100 | |
| 39.1 | 11* | 88 | 100 | |
| 19.5 | 7 | 92 | 100 | |
| 9.8 | 7 | 92 | 100 | |
| 4.9 | 3 | 96 | 100 | |
| 2.4 | 1 | 98 | 100 | |
| 1.2 | 12* | 87 | 100 | |
| 0.6 | 6 | 93 | 100 | |
| 0.3 | 7 | 92 | 100 | |
| 0.2 | 1 | 100 | 100 | |
| 0.1 | 3 | 96 | 100 | |
| 0.04 | 4 | 95 | 100 | |

Tables 19A-19B: Synergistic Growth Inhibition with Tasisulam in Combination with 1000 nM Compound A Across Two Wild Type Cell Lines

Synergy was measured across the Tasisulam titration with 1000 nM Compound A by Bliss Independence analysis. Bliss scores >10 are indicated by “*” and are considered synergistic. Bliss scores are valid for combinations exhibiting greater than 20% growth inhibition (GDI <80). Synergy as reported using Bliss may be underrepresented within cytotoxic dose ranges. Growth death index values are shown for the combination titration and each single agent Compound A (Cmpd A) and Tasisulam on a scale of −100 to 100 with the midpoint (0) being the number of cells at the beginning of the combination (T₀). Scores less than 0 are indicated by “**” and are representative of a cytotoxic response. Bliss and GDI values are shown for five MTAP-deficient cell lines: NCI-1H520 (19A), TCCSUP (191B).

TABLE 19A
NCI-H520

| | | | Single Agents | |
|---|---|---|---|---|
| | Tasisulam + 1 uM Cmpd A | | Tasisulam | Cmpd A |
| Dose (nM) | Excess Over Bliss | GDI | GDI | GDI |
| 20000 | 9 | 6 | 15 | |
| 10000 | 31* | 32 | 64 | |
| 5000 | 7 | 92 | 100 | |
| 2500 | −1 | 100 | 100 | |
| 1250 | −1 | 100 | 100 | 100 |
| 625 | 1 | 100 | 100 | |
| 312.5 | −2 | 100 | 100 | |
| 156.3 | 2 | 100 | 100 | |
| 78.1 | 0 | 100 | 100 | |
| 39.1 | −3 | 100 | 100 | |
| 19.5 | −1 | 100 | 100 | |
| 9.8 | 1 | 98 | 100 | |
| 4.9 | 3 | 96 | 100 | |
| 2.4 | 1 | 96 | 100 | |
| 1.2 | −1 | 100 | 100 | |
| 0.6 | 13* | 86 | 100 | |
| 0.3 | 6 | 96 | 100 | |
| 0.2 | −1 | 100 | 100 | |
| 0.1 | −1 | 100 | 100 | |
| 0.04 | 6 | 93 | 100 | |

TABLE 19B
TCCSUP

| | | | Single Agents | |
|---|---|---|---|---|
| | Tasisulam + 1 uM Cmpd A | | Tasisulam | Cmpd A |
| Dose (nM) | Excess Over Bliss | GDI | GDI | GDI |
| 20000 | 17* | 46 | 75 | |
| 10000 | 20* | 59 | 94 | |
| 5000 | 16* | 68 | 100 | |
| 2500 | 5 | 79 | 100 | |
| 1250 | −3 | 87 | 100 | 84 |
| 625 | −3 | 87 | 100 | |
| 312.5 | 4 | 80 | 100 | |
| 156.3 | 4 | 80 | 100 | |
| 78.1 | 4 | 80 | 100 | |
| 39.1 | 3 | 80 | 100 | |
| 19.5 | 2 | 82 | 100 | |
| 9.8 | −1 | 85 | 100 | |
| 4.9 | 1 | 83 | 100 | |
| 2.4 | −1 | 84 | 100 | |
| 1.2 | 6 | 78 | 100 | |
| 0.6 | 1 | 83 | 100 | |
| 0.3 | 4 | 80 | 100 | |
| 0.2 | 3 | 81 | 100 | |
| 0.1 | 7 | 77 | 100 | |
| 0.04 | 4 | 80 | 100 | |

### Example 6: MAT2A and Topoisomerase Inhibitors Provide Combination Benefit in In Vitro MTAP-Deficient Models

### Materials and Methods

A panel of 13 MTAP-deficient non-small cell lung cancer (NSCLC), bladder, pancreatic, gastric, esophageal, and head and neck squamous cell carcinoma (HNSCC) cancer cell lines was used to assess the combinatory effect of a MAT2A inhibitor (Compound A) and topoisomerase inhibitors: 10-hydroxycamptothecin, irinotecan, topotecan, daunorubicin, doxorubicin, and etoposide. To determine the optimal concentration range of each compound in each cell line for the combination screen, cells were seeded at 150 cells/well for Compound A or 500 cells/well for Topoisomerase inhibitors in a 384-well plate. After 24 hrs, cells were treated with a 9-point titration of each compound as a single agent and incubated at 37° C. with 5% CO₂ for 6 days. For the combination screen, cells were seeded at 150 cells/well in a 384-well plate. After 24 hrs, cells were co-treated with a 5-point titration of each compound in an optimized 6×6 dose matrix and incubated at 37° C. with 5% CO₂ for 6 days. Cells were subsequently lysed with Promega CellTiter-Glo (CTG) 2.0 reagent according to the manufacturer's protocol and the chemiluminescent signal was measured using an EnVision plate reader (PerkinElmer) for cell number quantification. A plate of untreated cells was harvested at the time of compound addition (To or time zero) for cell number quantification using CTG. Each data point was run in technical triplicate.

The percent of growth inhibition is calculated as:

$$If T < {V}_{0} : 100 * ( 1 - ( T - {V}_{0} ) / {V}_{0} )$$

$$If T \underline{>} {V}_{0} : 100 * ( 1 ( T - {V}_{0} ) / ( V - {V}_{0} ) )$$

where T is the signal measure for a test article, V is the vehicle-treated control measure, and V₀ is the vehicle control measure at T₀. The percent growth inhibition is used to generate dose-response curves and GI₅₀ calculations for single drug activity and drug combination synergy using the Chalice Analyzer software (Horizon). 100% and >100% growth inhibition represented cytostasis and cytotoxicity, respectively.

Synergistic growth inhibition was assessed using the Loewe additivity model. The observed growth inhibition at each dose was compared to the predicted inhibition based on the additive activity of either drug combined with itself. The calculation for Loewe additivity is: I_{Loewe} that satisfies (X/X_{I})+(Y/Y_{I})=1, where X_{I} and Y_{I} are the single agent effective concentrations for the observed combination effect I. A difference between the observed and predicted growth inhibition ≥20% was considered synergistic.

A Synergy Score was calculated to quantify the strength of synergy as following:

$$Synergy Score = log {f}_{X} log {f}_{Y} \sum max ( 0 , {I}_{d a t a} ) ( {I}_{d a t a} - {I}_{Loewe} )$$

The fractional inhibition for each component agent and combination point in the matrix is calculated relative to the median of all untreated/vehicle-treated control wells. A Synergy Score of >2.22 was considered synergistic, given that it corresponded with high growth inhibition and ≥20% excess over Loewe additivity model at multiple dose points.

### Results

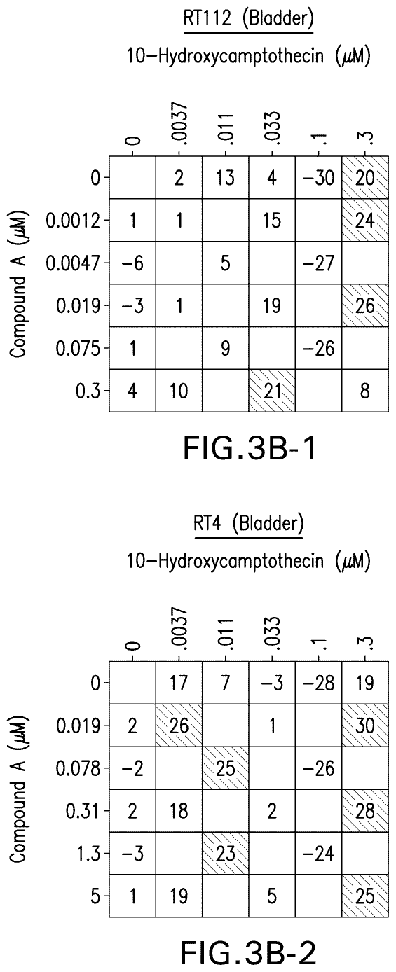

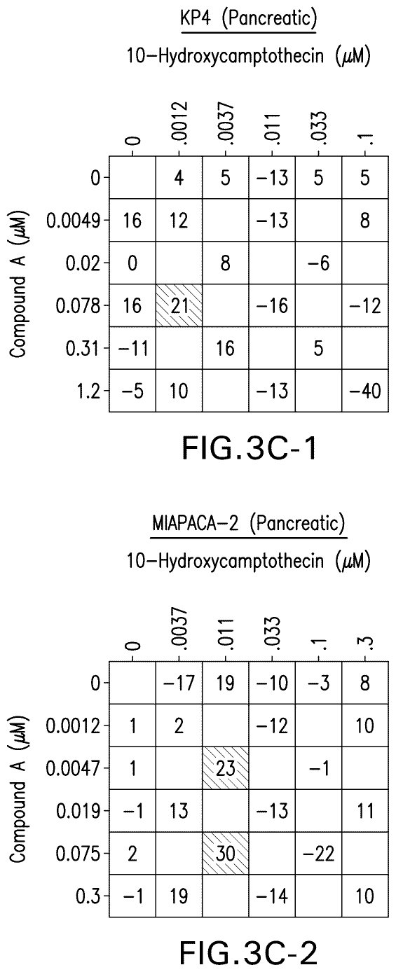

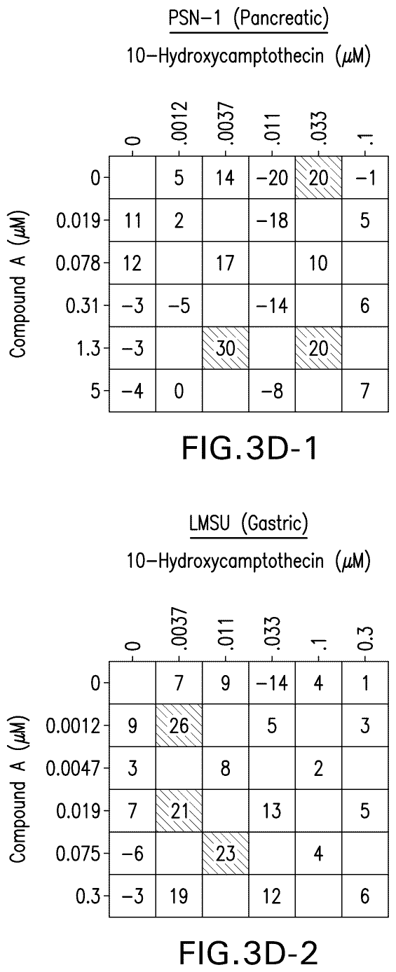

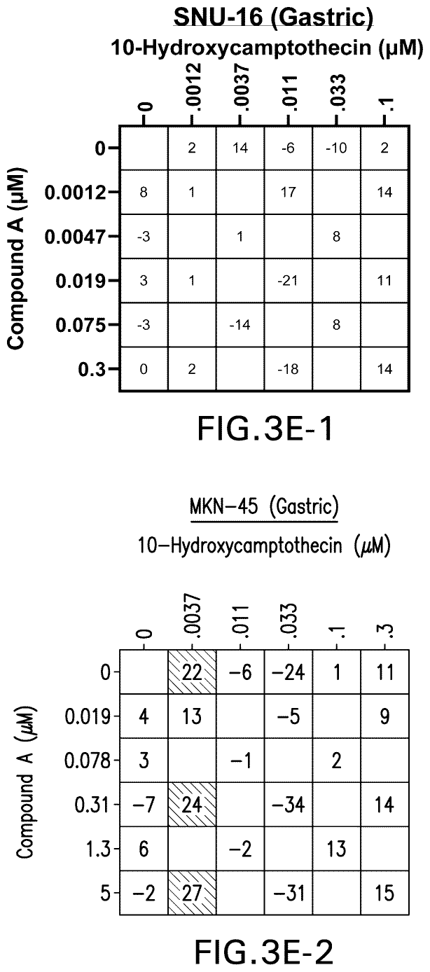

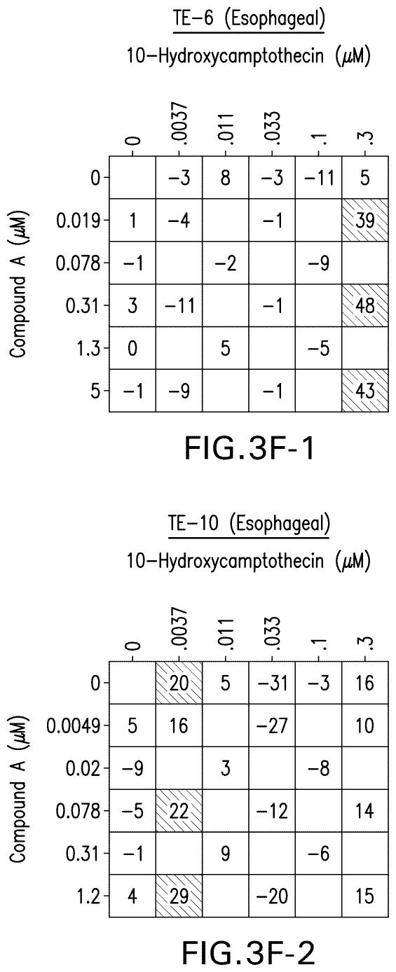

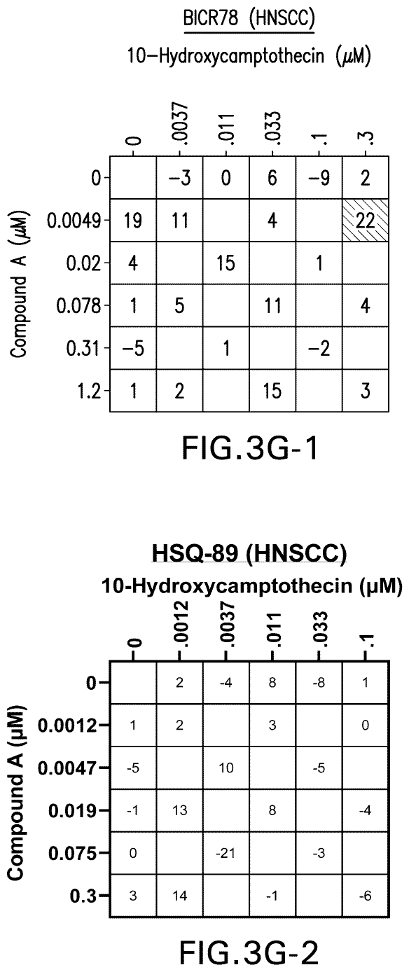

The combination of Compound A and 6 different topoisomerase inhibitors was tested in a panel of 13 MTAP-deficient cell lines. Synergistic growth inhibition was observed with each combination, but to varying degrees across the cell line panel. For instance, the combination of Compound A with 10-hydroxycamptothecin enhanced the growth inhibition observed compared to either single agent and showed strong synergy (as determined by a synergy score of >2.22) in 10/13 MTAP-deficient cell lines (FIG. 2A-1 to FIG. 2G, FIG. 3A to FIG. 3G-2, and Table 20).

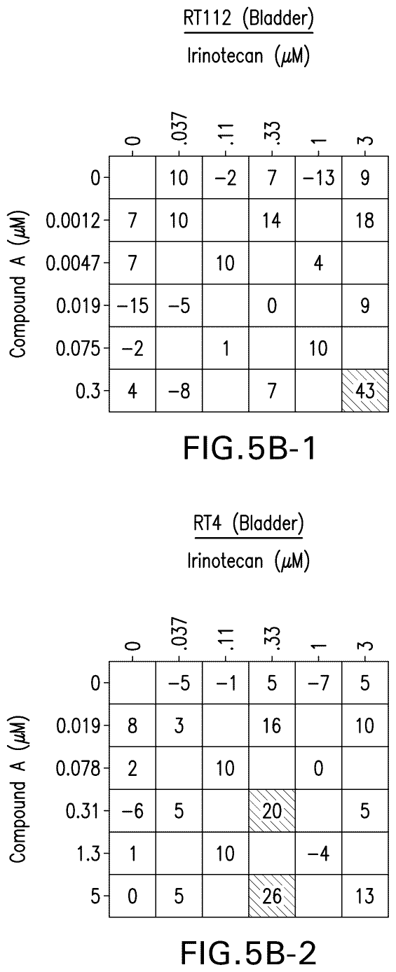

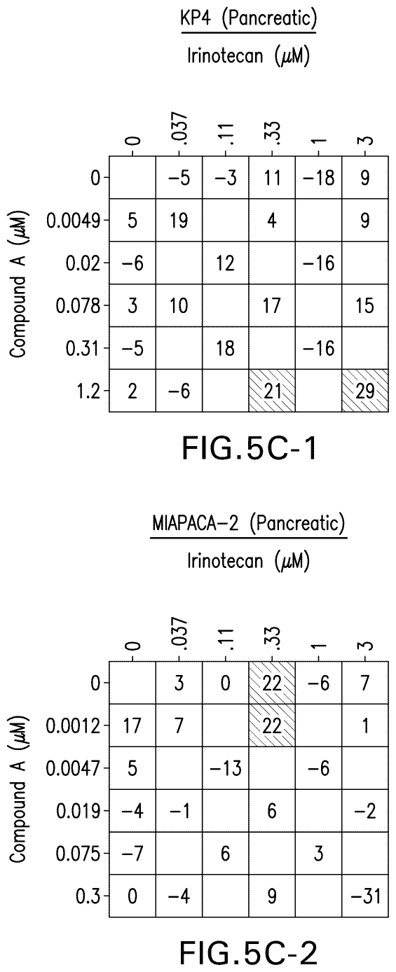

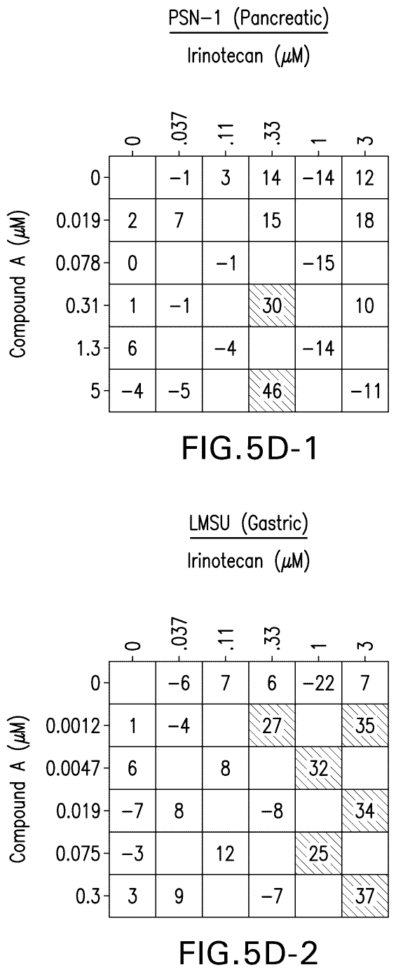

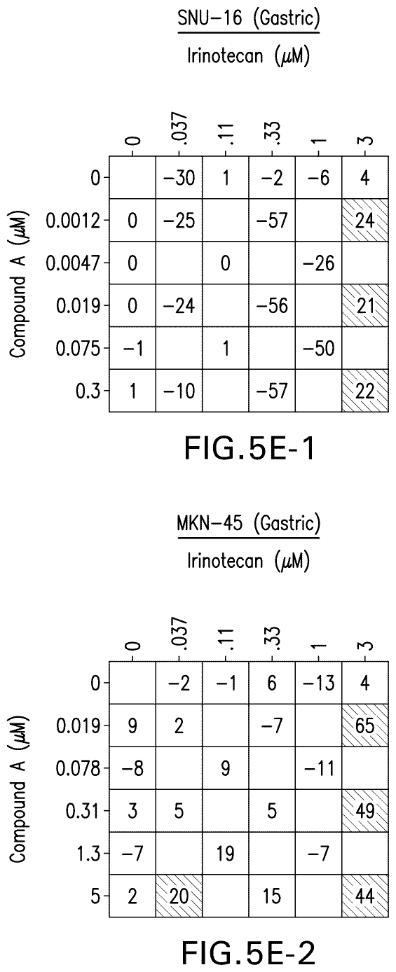

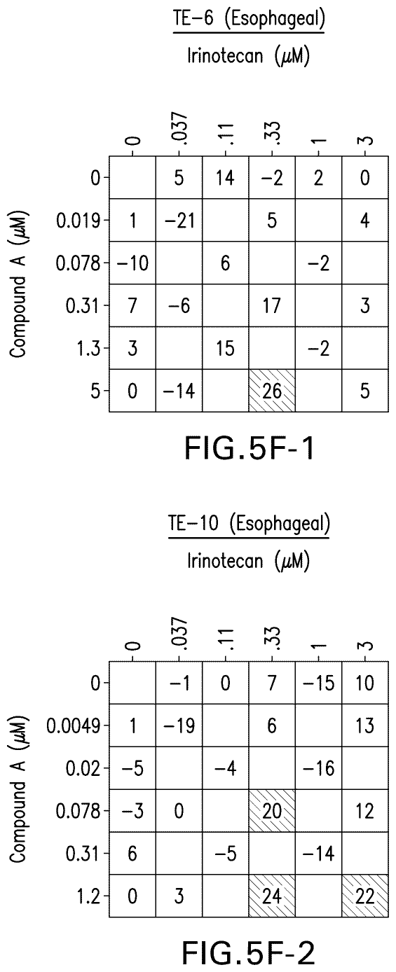

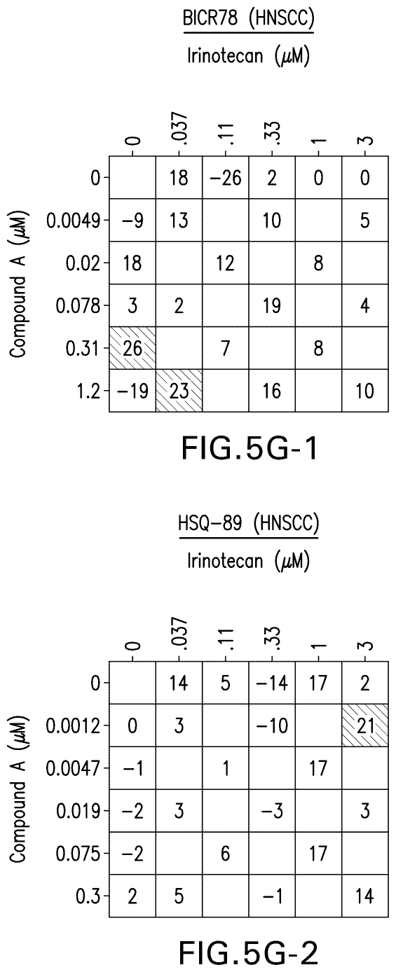

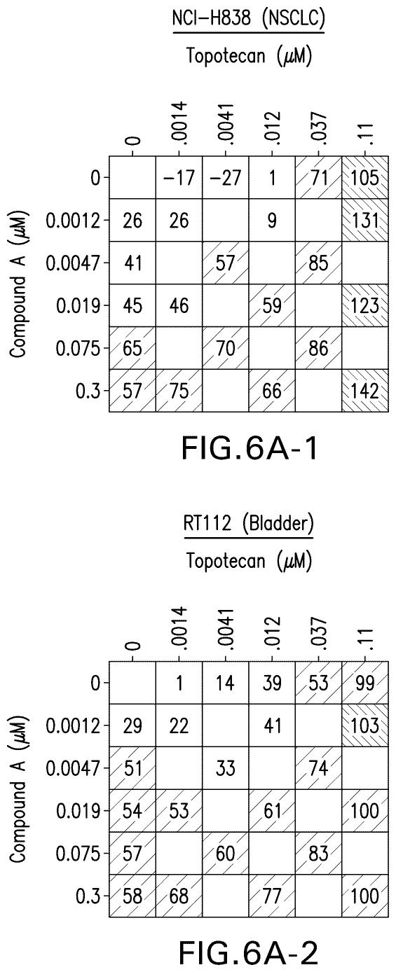

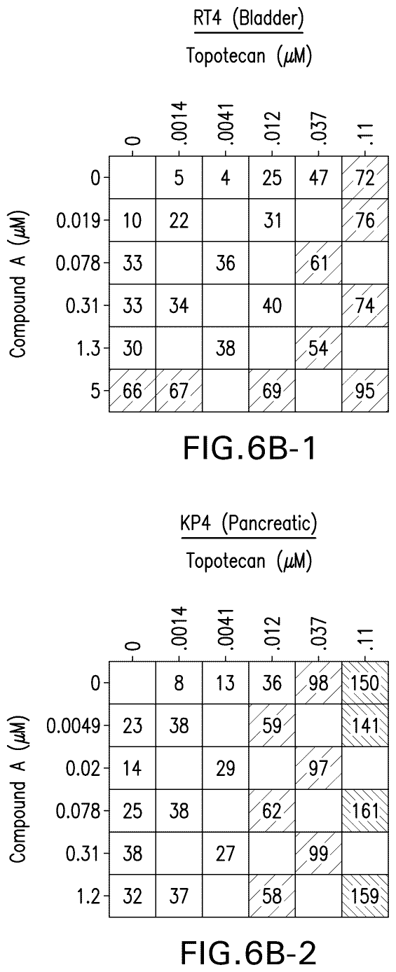

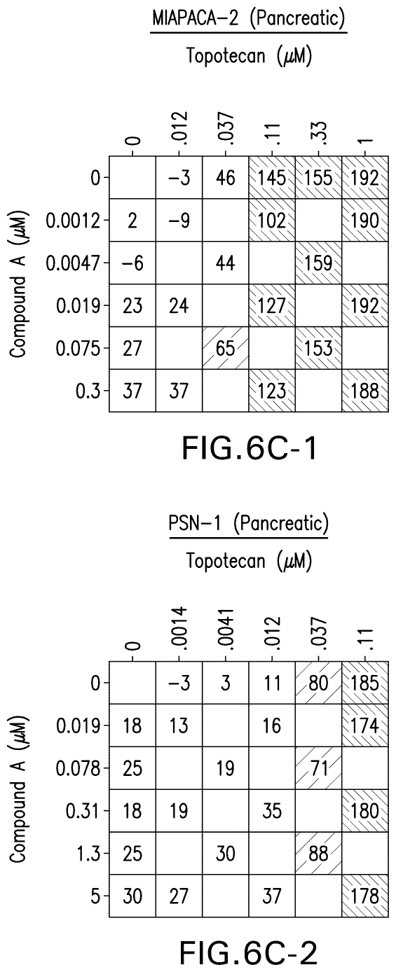

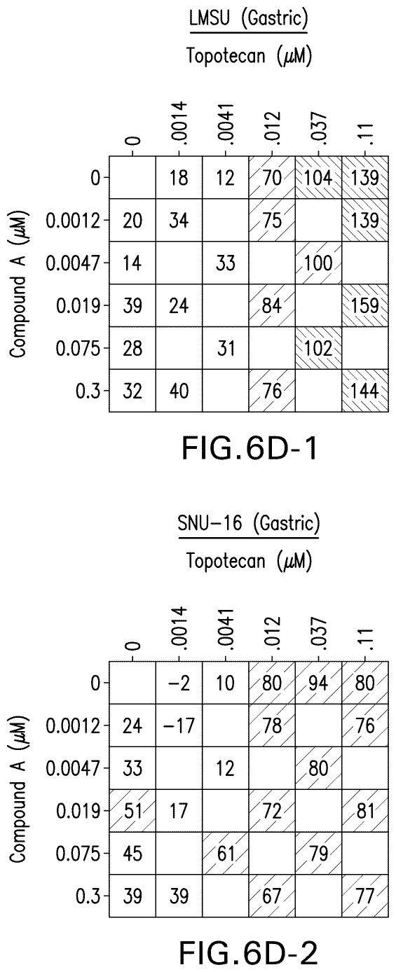

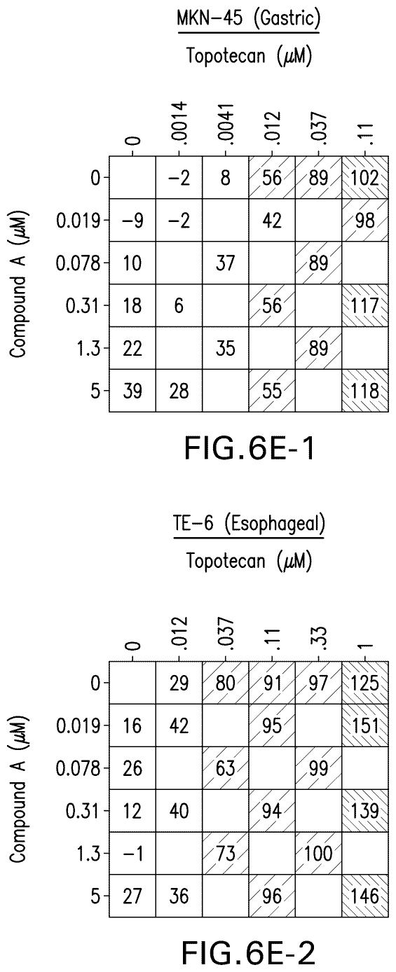

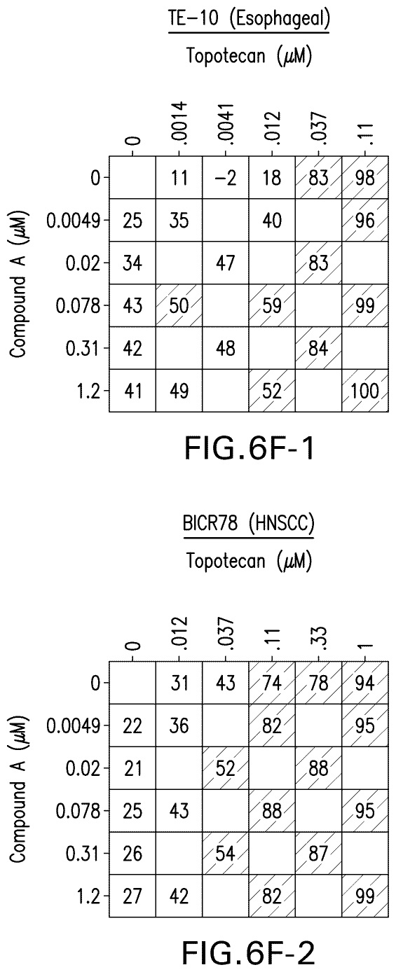

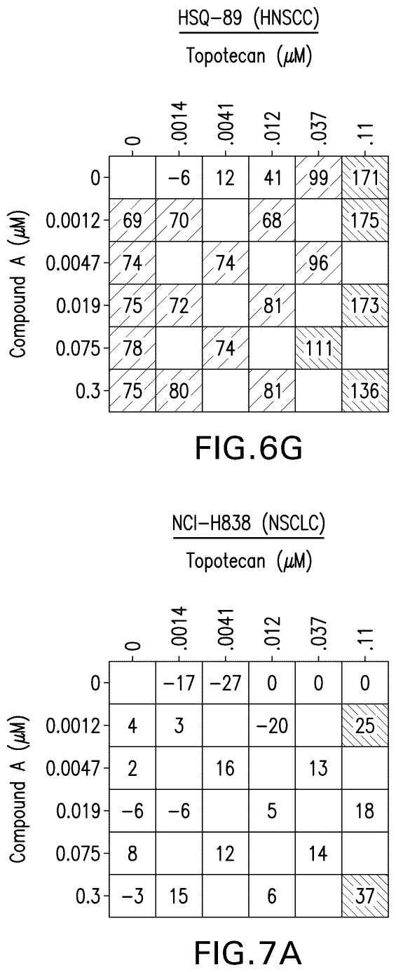

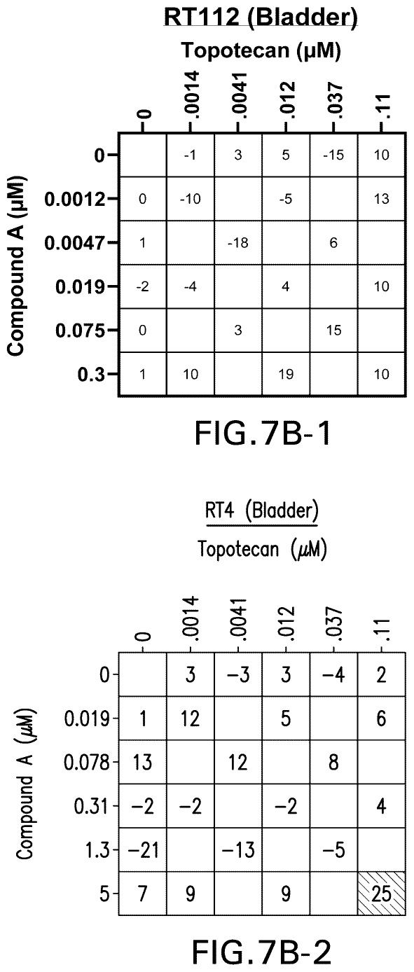

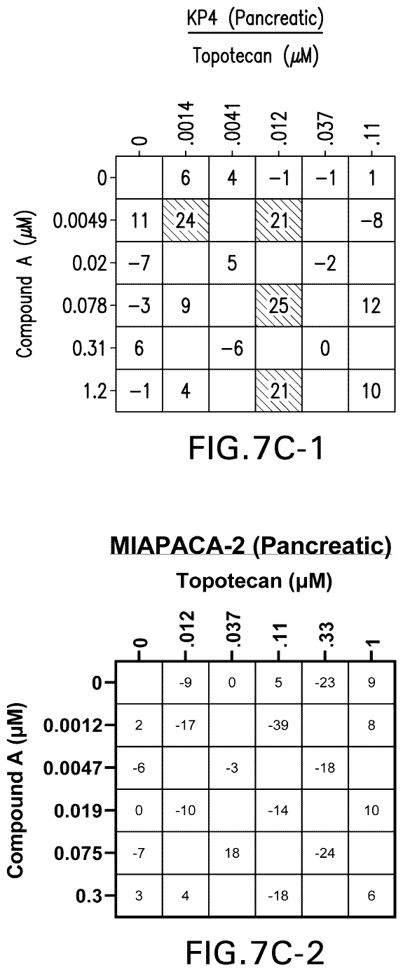

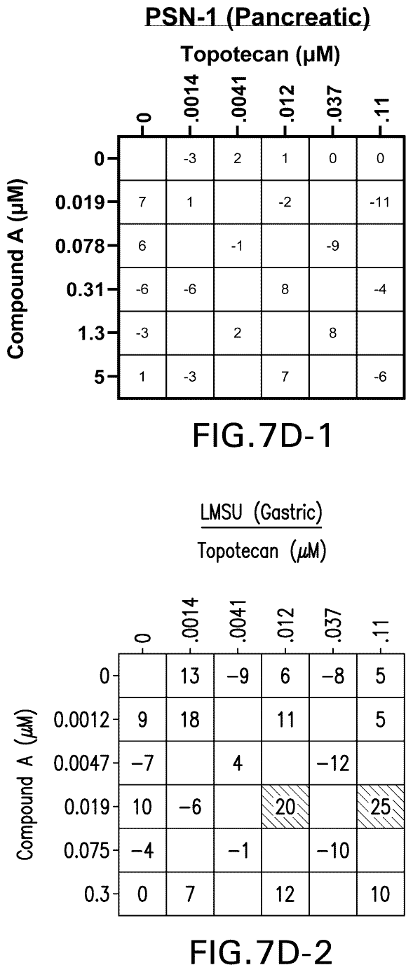

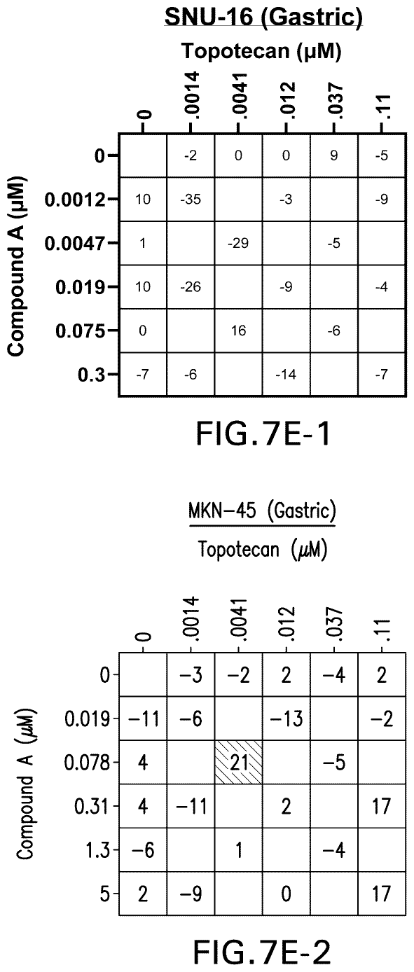

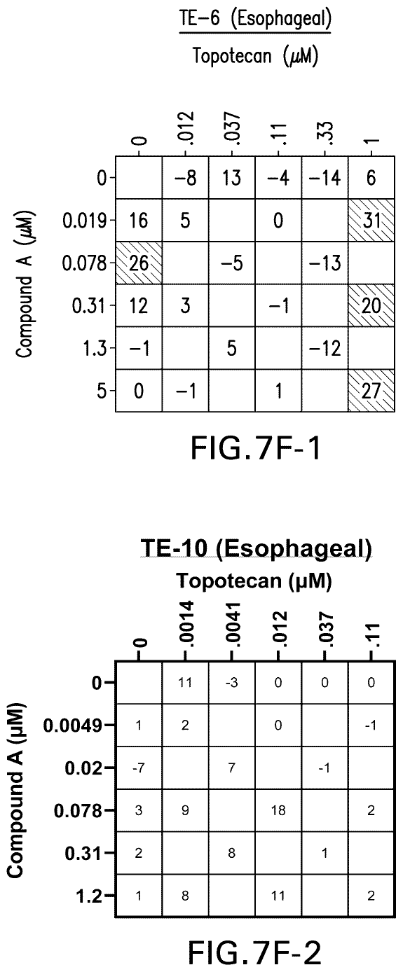

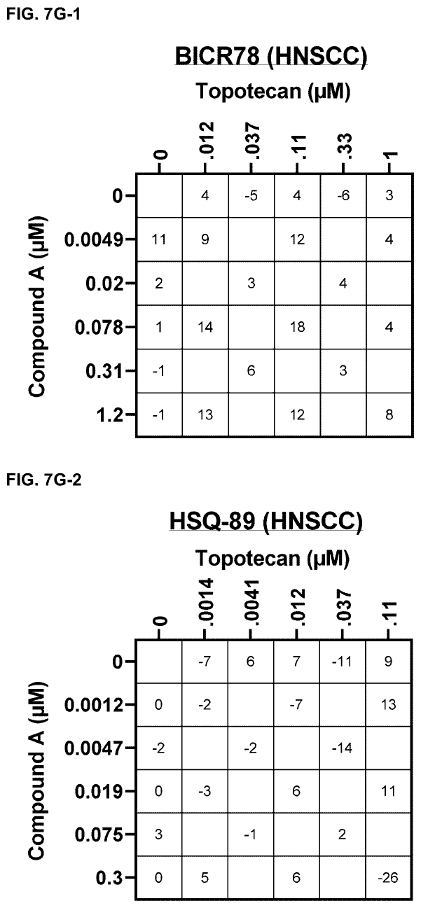

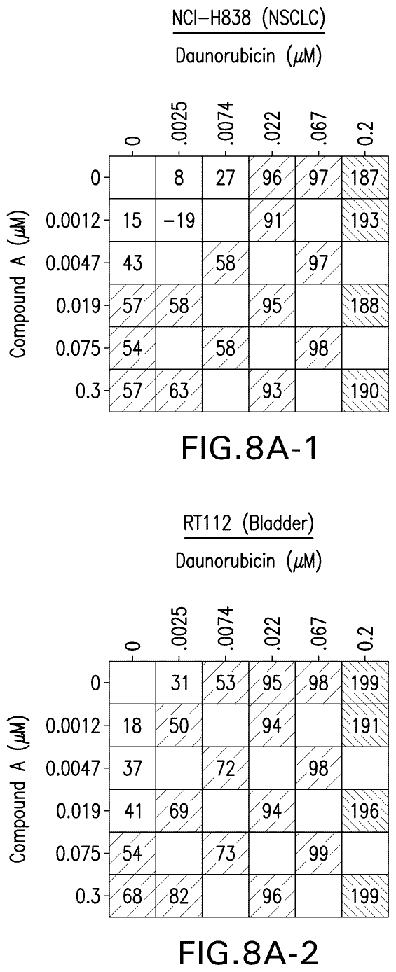

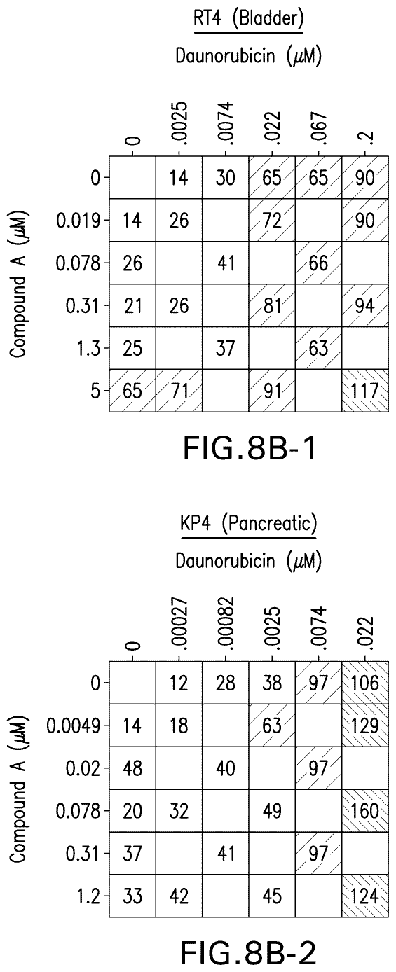

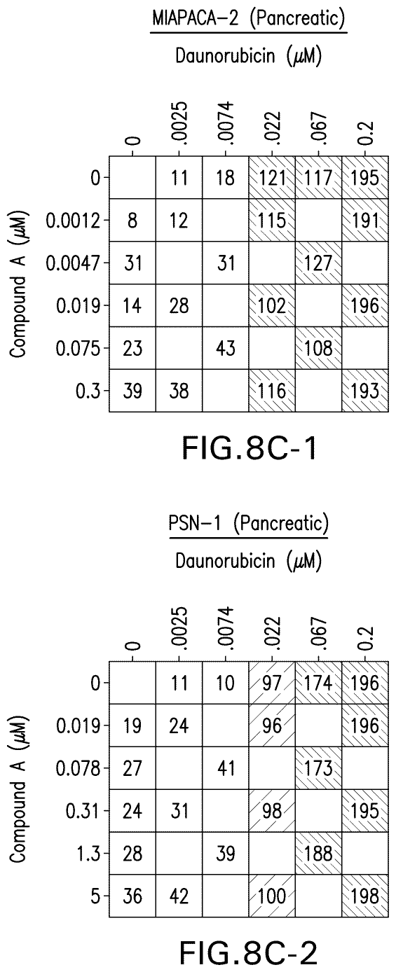

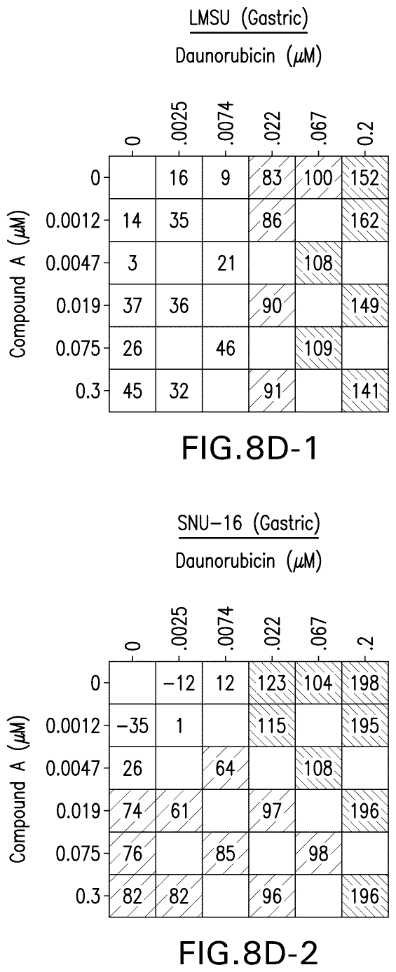

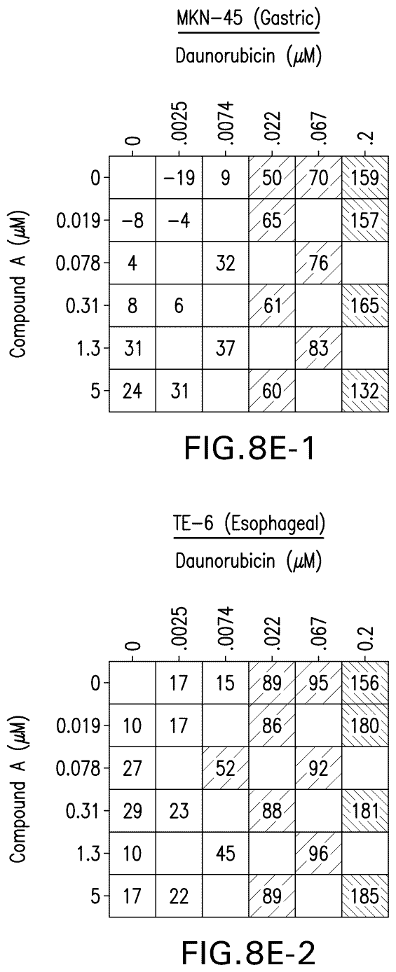

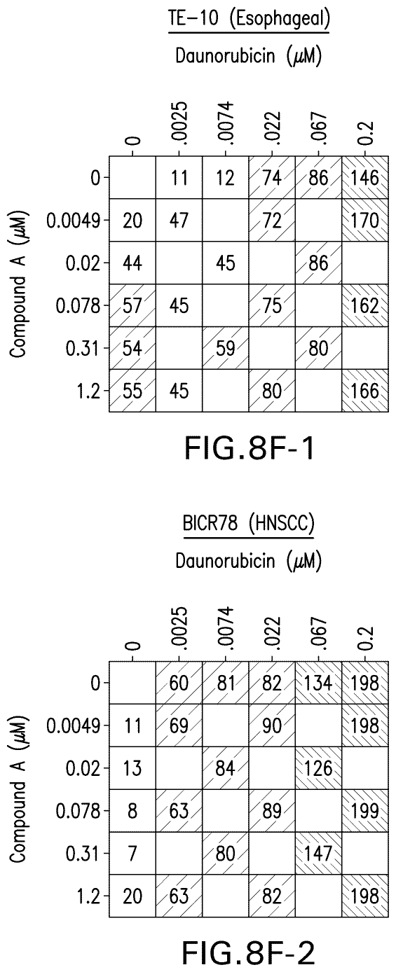

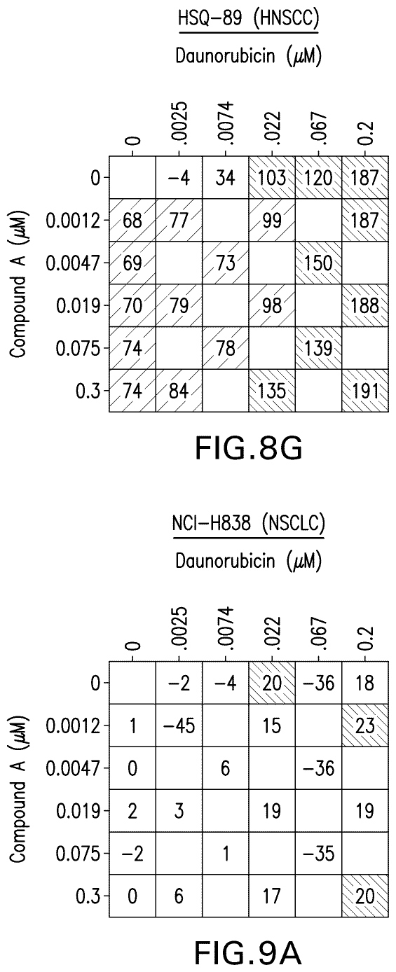

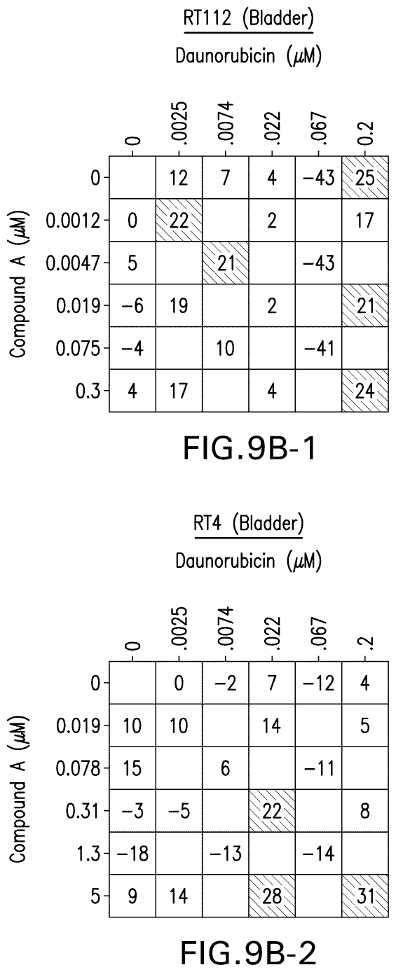

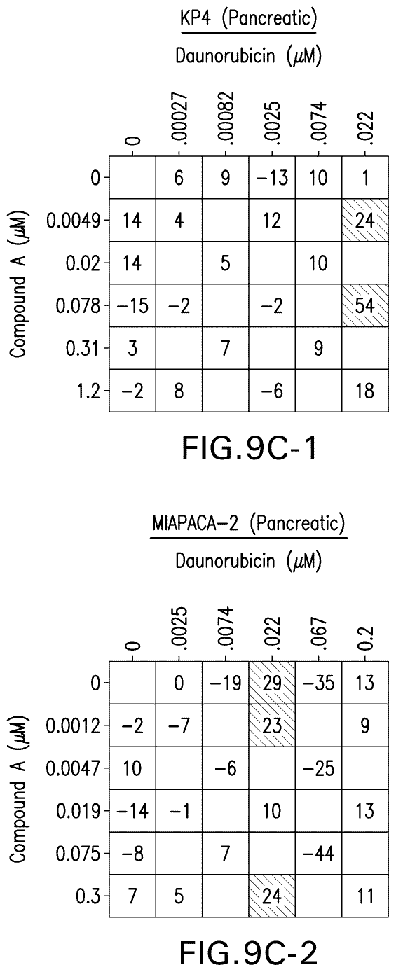

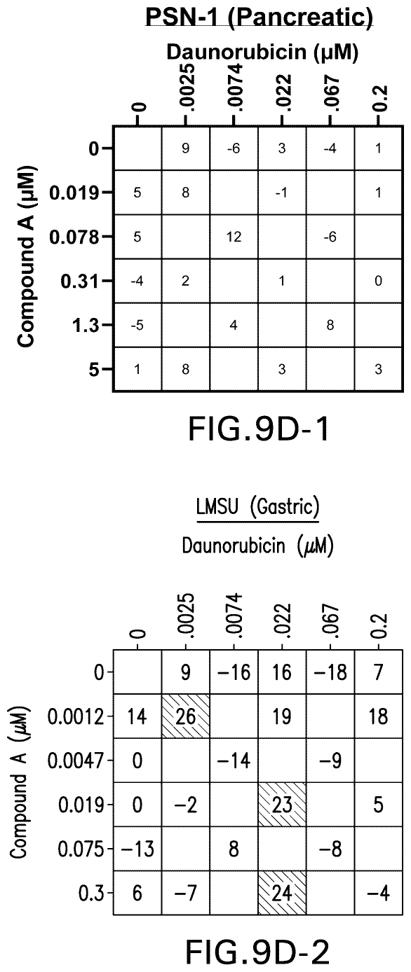

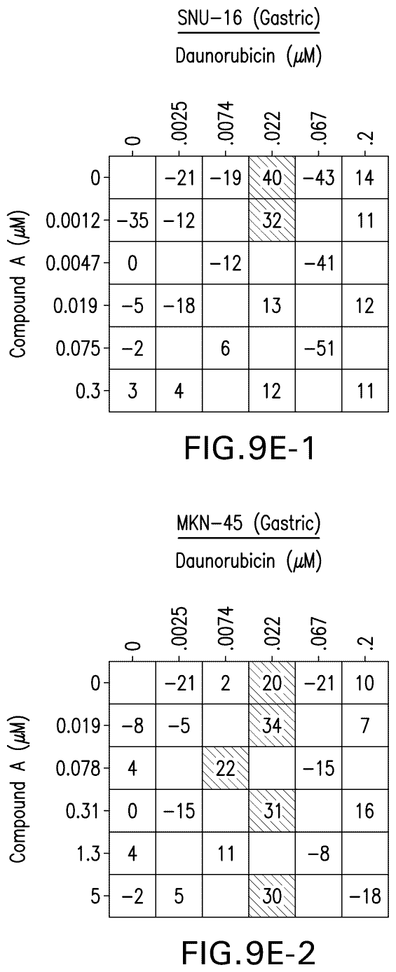

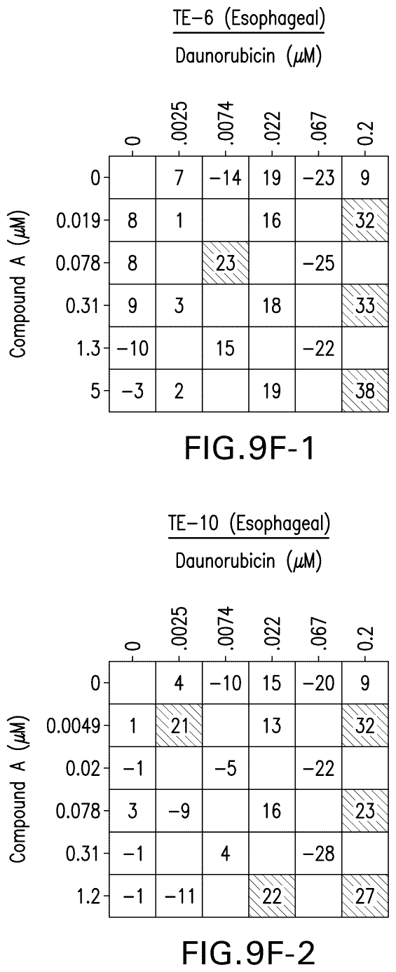

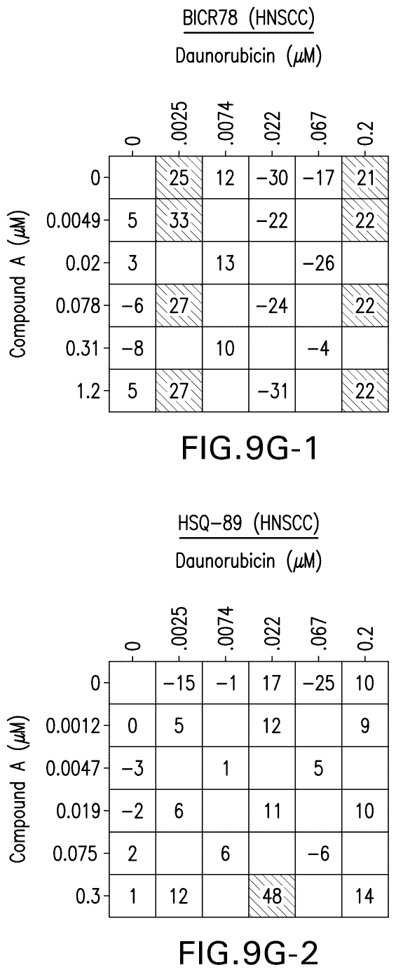

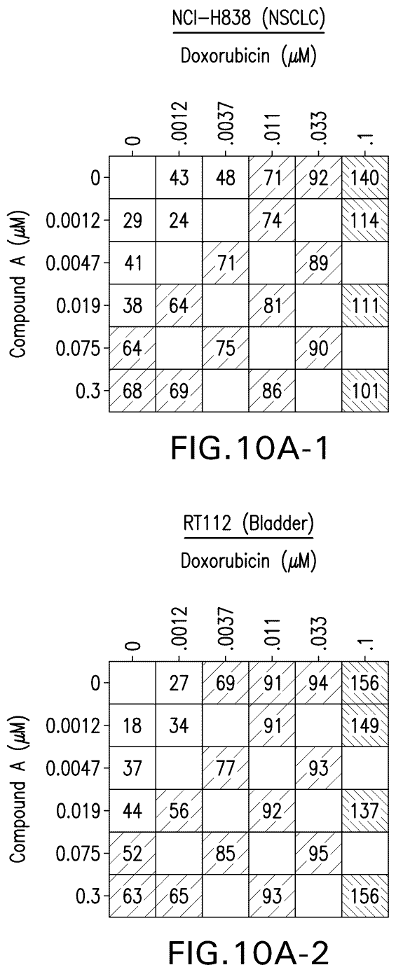

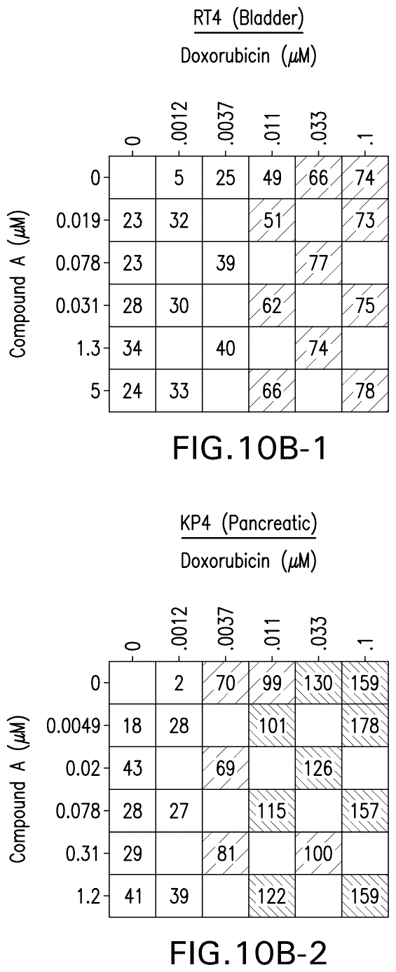

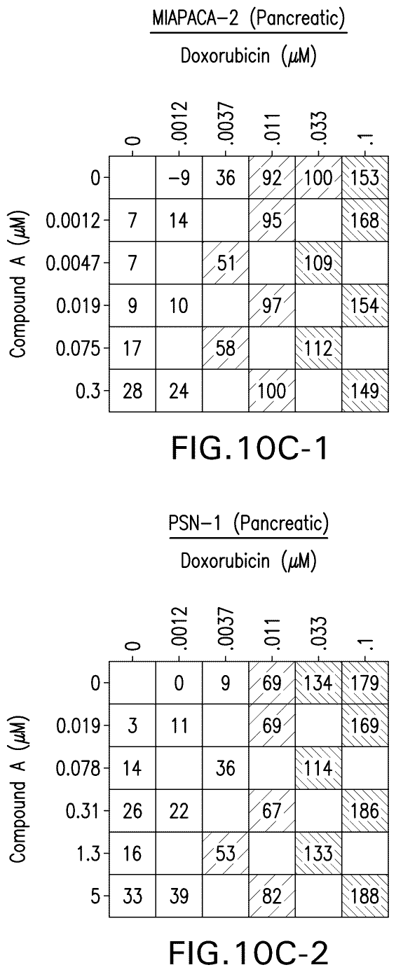

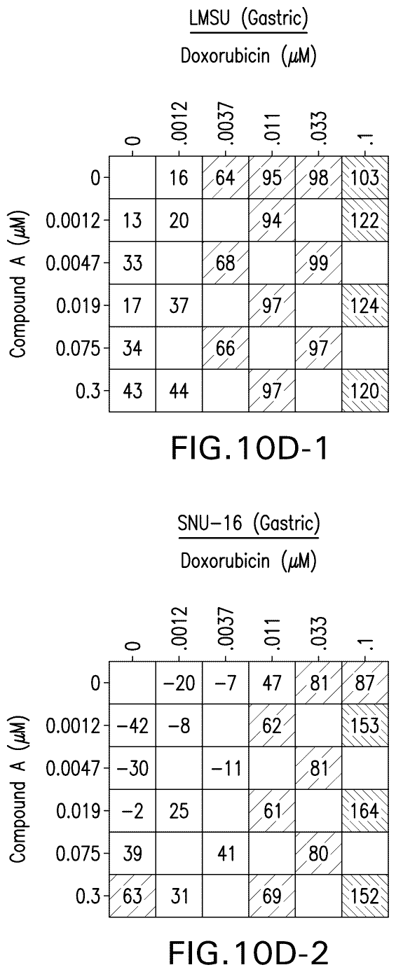

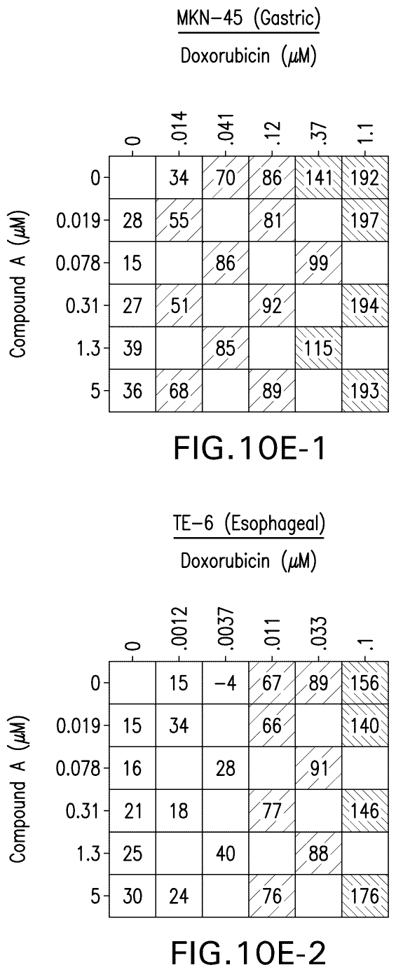

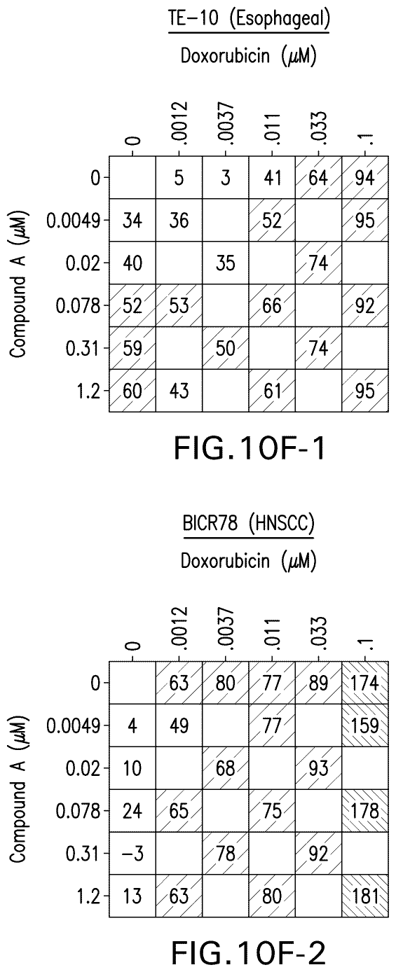

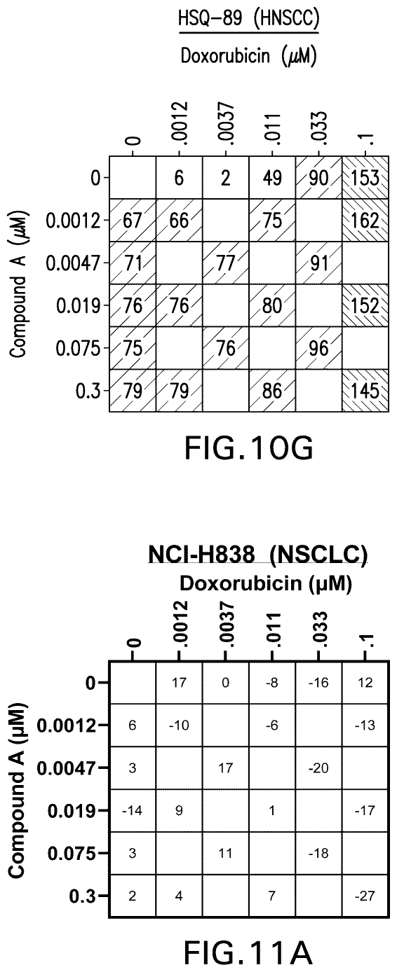

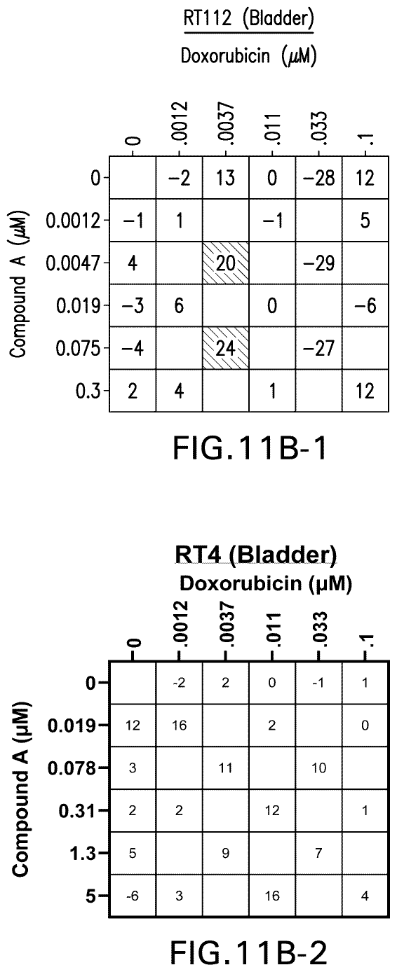

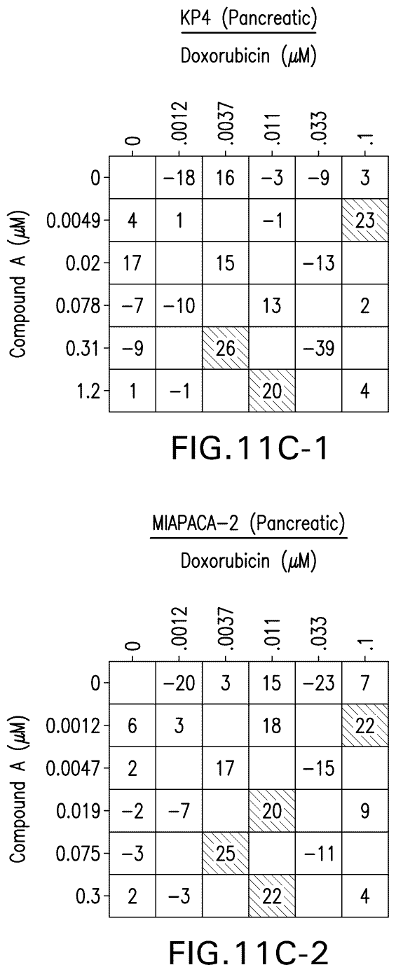

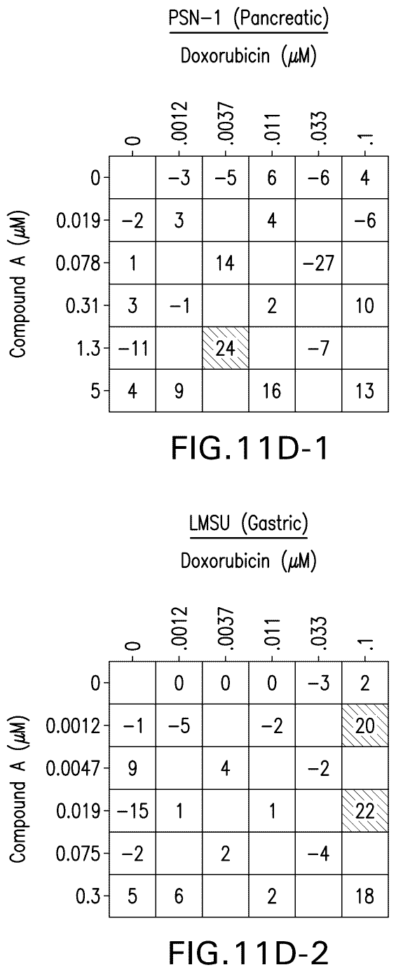

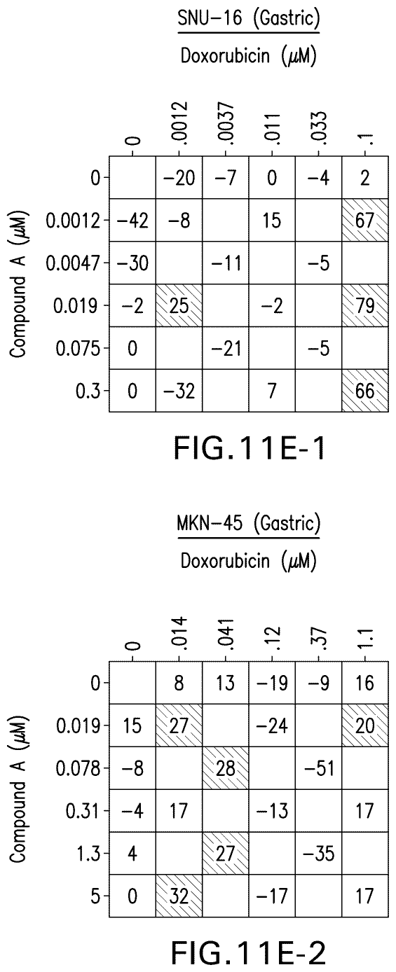

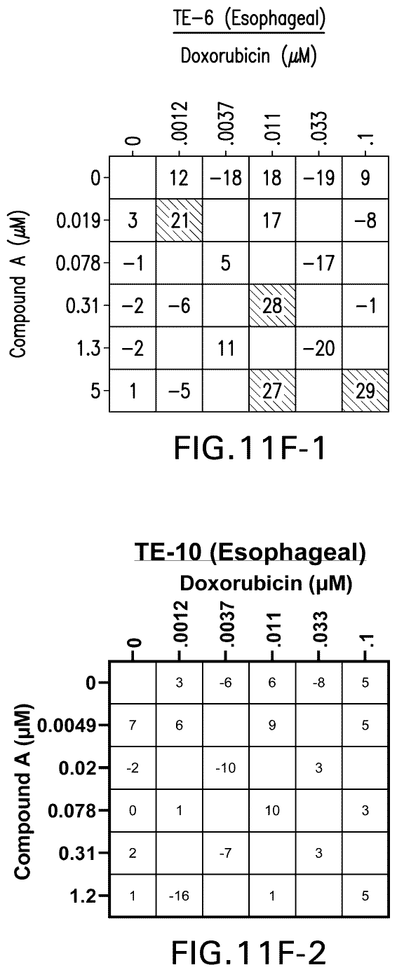

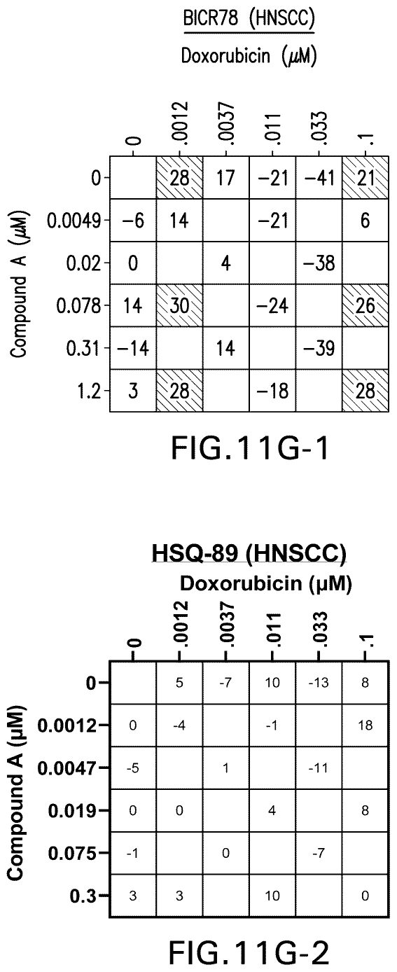

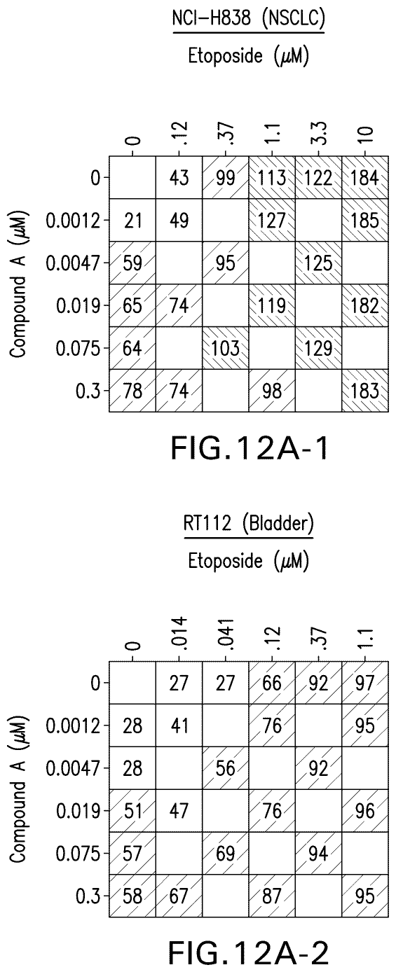

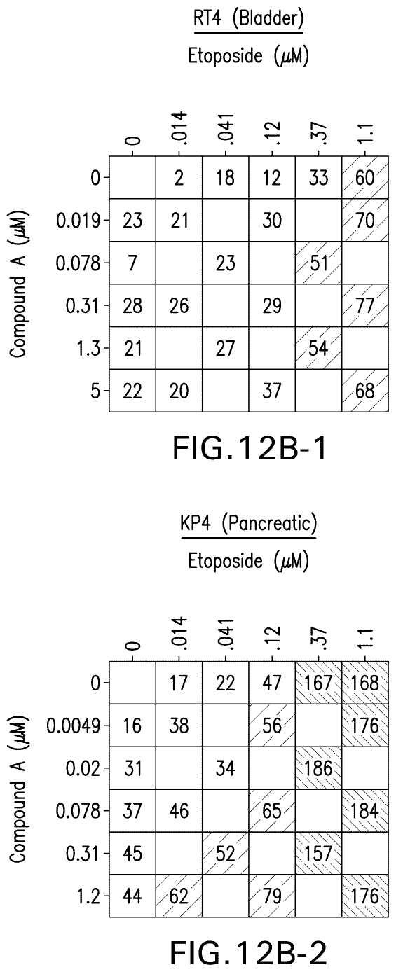

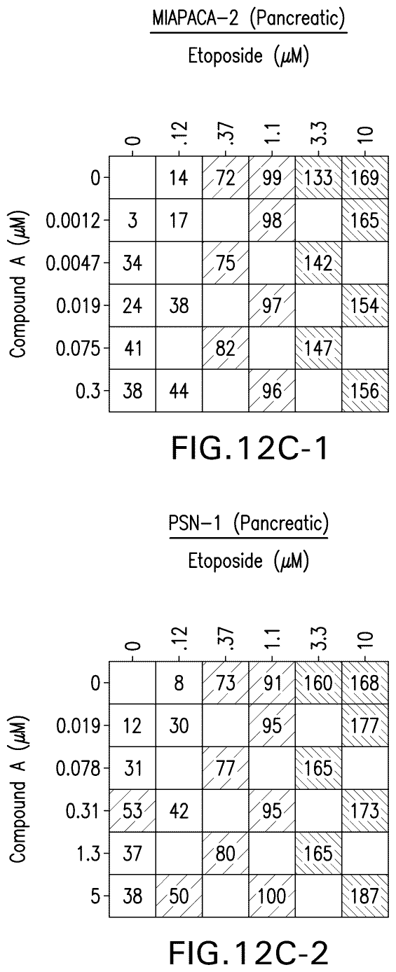

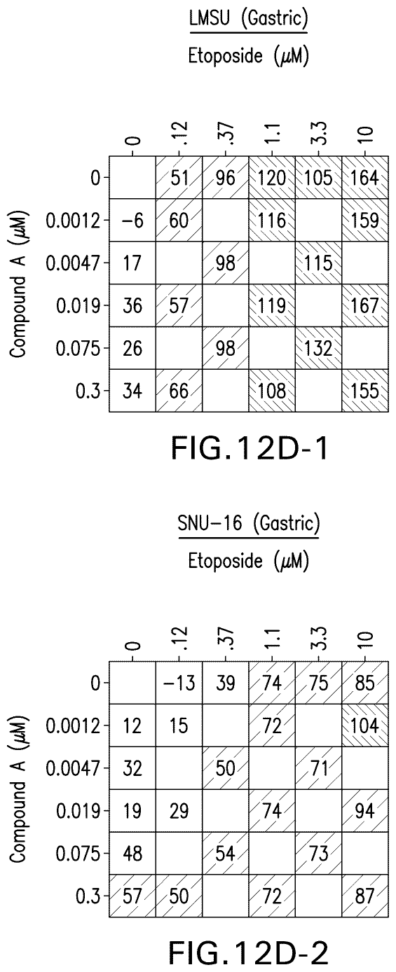

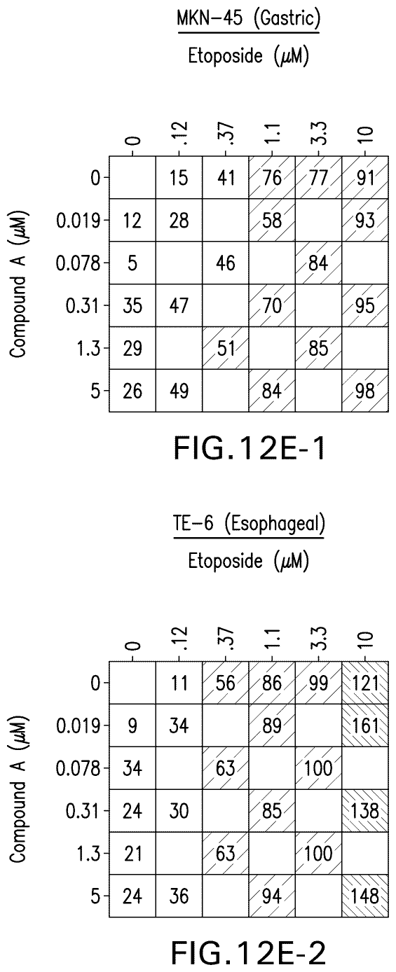

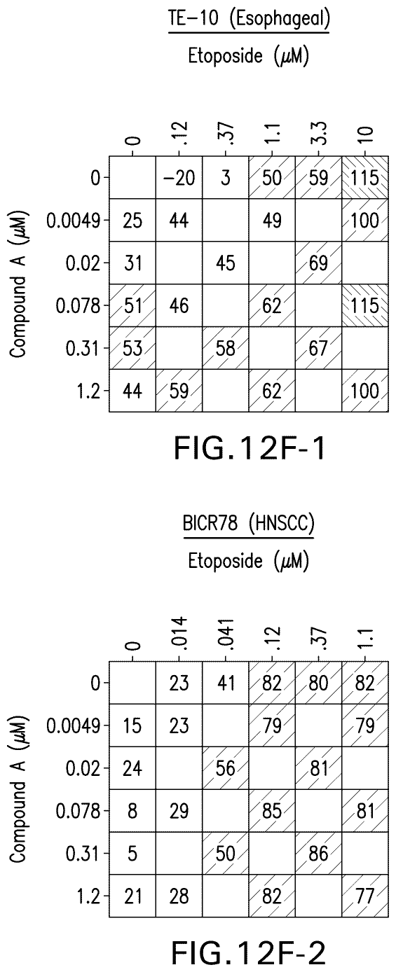

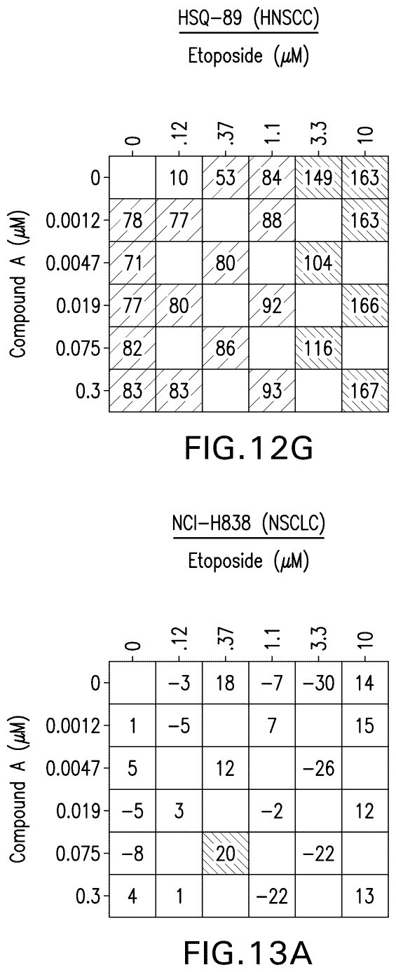

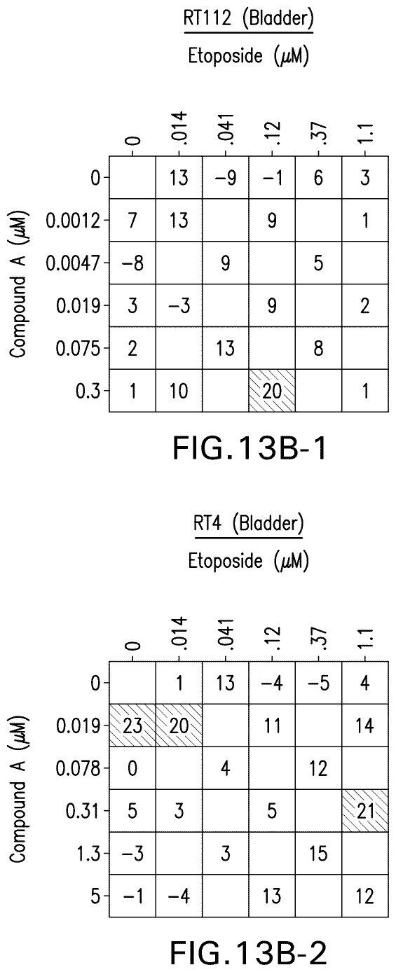

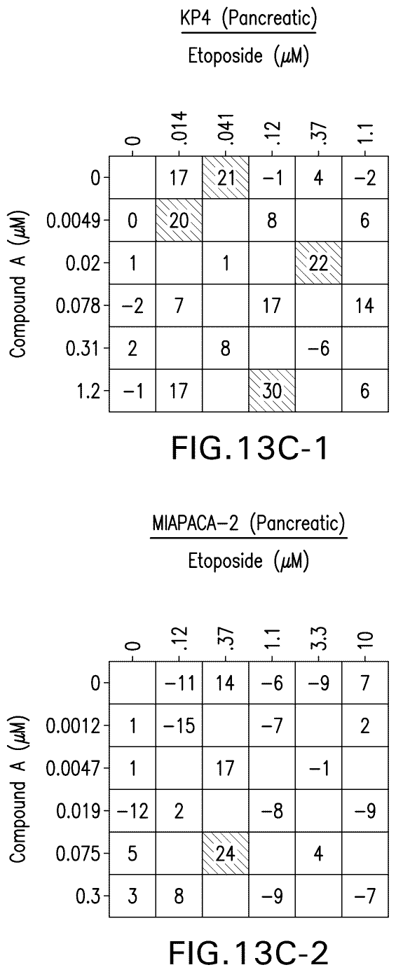

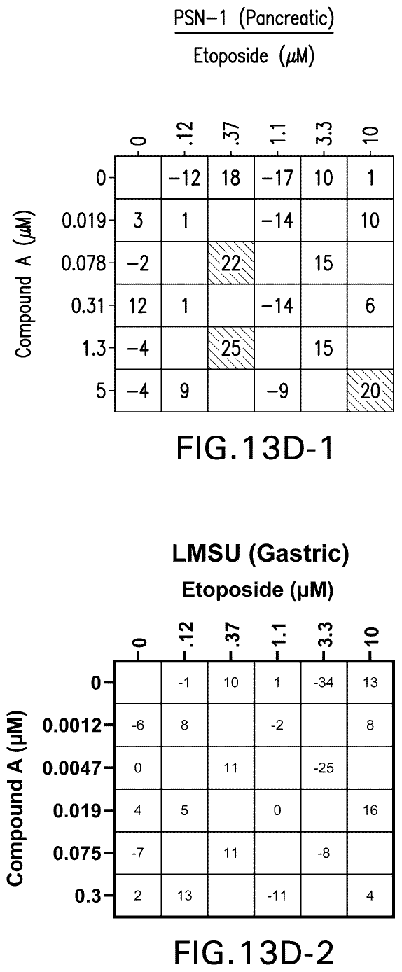

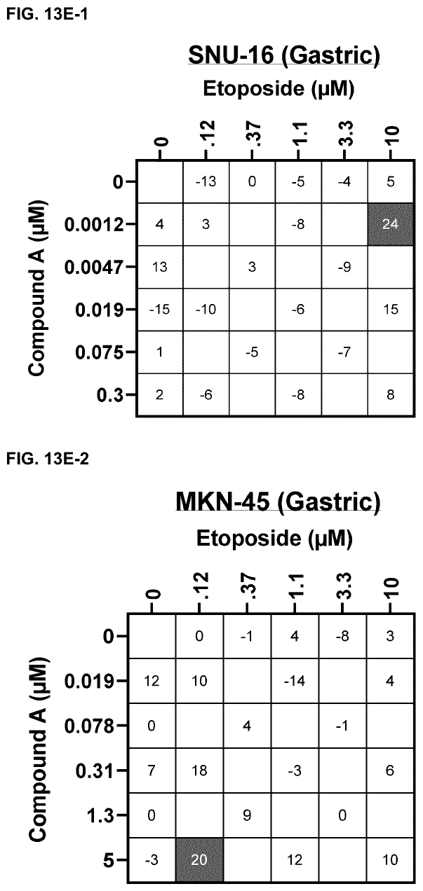

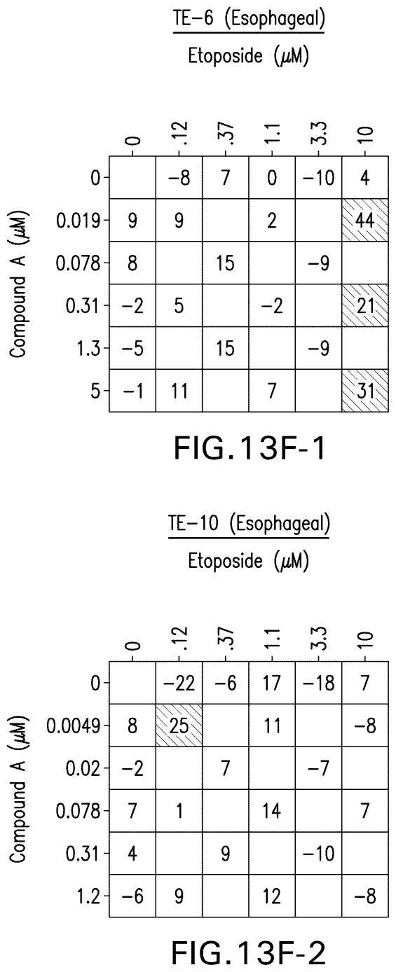

Combination of Compound A with irinotecan also showed enhanced growth inhibition compared to single agents and showed synergy in 8/13 MTAP-deficient cell lines (FIG. 4A-1 to FIG. 4G, FIG. 5A to FIG. 5G-2, and Table 20). The combination of Compound A and topotecan also enhanced the growth inhibition observed with either single agent and showed synergy in 6/13 MTAP-deficient cell lines (FIG. 6A-1 to FIG. 6G, FIG. 7A to FIG. 7G-2, and Table 20). The combination of Compound A and daunorubicin showed enhanced growth inhibition and strong synergy across 12/13 MTAP-deficient cell lines (FIG. 8A-1 to FIG. 8G, FIG. 9A to FIG. 9G-2, and Table 20). Combination of Compound A with doxorubicin showed enhanced growth inhibition and strong synergy in 8/13 MTAP-deficient cell lines (FIG. 10A-1 to FIG. 10G, FIG. 11A to FIG. 11G-2, and Table 20). The combination of Compound A and etoposide showed enhanced growth inhibition and strong synergy in 5/13 MTAP-deficient cell lines (FIG. 12A-1 to FIG. 12G, FIG. 13A to FIG. 13G-2, and Table 20).

In growth inhibition figures, growth inhibition is presented as a percent of TO for each cell line in a 6×6 dose matrix. 50-100% growth inhibition is indicated by “*”, with 100% growth inhibition representing cytostasis. Greater than 100% growth inhibition is highlighted in dark gray and represents cytotoxicity. In Loewe synergy figures, synergy is measured at all tested concentrations of Compound A and 10-Hydroxycamptothecin using the Loewe additivity model for each cell line. Values >20 are indicated by “**” and are considered synergistic.

TABLE 20
Synergy scores for the combination of Compound A and either 10-Hydroxycamptothecin (10-HCPT),
Irinotecan, Topotecan, Daunorubicin, Doxorubicin, or Etoposide in 13 MTAP-deficient cell lines.

| | | Synergy Scores | | | | | |
|---|---|---|---|---|---|---|---|
| | | Cmpd A × | Cmpd A × | Cmpd A × | Cmpd A × | Cmpd A × | Cmpd A × |
| Cell Line | Indication | 10-HCPT | Irinotecan | Topotecan | Daunorubicin | Doxorubicin | Etoposide |
| NCI-H838 | NSCLC | 5.55 | 2.16 | 4.90 | 5.11 | 1.03 | 3.41 |
| RT112 | Bladder | 4.96 | 3.47 | 2.23 | 5.61 | 2.00 | 2.14 |
| RT4 | Bladder | 6.18 | 1.93 | 1.67 | 3.41 | 1.51 | 1.90 |
| KP4 | Pancreatic | 1.91 | 4.50 | 2.61 | 5.07 | 3.5 | 4.48 |
| MIAPACA-2 | Pancreatic | 3.39 | 0.617 | 1.70 | 3.84 | 4.02 | 1.32 |
| PSN-1 | Pancreatic | 3.54 | 3.31 | 0.384 | 1.21 | 2.40 | 4.58 |
| LMSU | Gastric | 6.34 | 9.56 | 3.09 | 3.16 | 2.47 | 2.47 |
| SNU-16 | Gastric | 3.81 | 3.59 | 0.275 | 4.00 | 10.4 | 1.39 |
| MKN-45 | Gastric | 4.48 | 8.62 | 1.43 | 3.21 | 5.8 | 1.66 |
| TE-6 | Esophageal | 6.00 | 1.18 | 3.55 | 7.47 | 3.42 | 5.29 |
| TE-10 | Esophageal | 4.05 | 3.28 | 1.08 | 5.52 | 0.924 | 1.61 |
| BICR78 | HNSCC | 2.04 | 1.20 | 2.26 | 6.06 | 4.75 | 1.07 |
| HSQ-89 | HNSCC | 1.67 | 3.95 | 1.69 | 5.36 | 1.65 | 0.829 |

Synergy Score values >2.22 are considered synergistic.

### Example 7: Antti-Tumor Activity of Compound A and Irinotecan in RT112/84 Mouse Xenograft Model

All mouse studies were approved by the Institutional Animal Care and Use Committee (IACUC) based on guidelines from the National Institutes of Health (NIH). Mice were maintained under pathogen-free conditions, and food and water were provided ad libitum.

The anti-tumor effect of Compound A and irinotecan hydrochloride as single-agents or in combination was assessed in the RT112/84 human bladder tumor cell-derived xenograft (CDX) model. Cells were expanded in RPMI-1640 (Gibco, Catalog Number 11875093) with 10% fetal bovine serum. These cells were free of mycoplasma and authenticated by STR profiling. Five million cells in log growth phase were resuspended in Hanks Balanced Salt Solution containing 50% Matrigel and implanted subcutaneously into the flank of each recipient female Crl:NU-Foxnlnu mouse. Mice were housed in microisolator cages with corn cob bedding with additional enrichment consisting of sterile nesting material (Innovive) and Bio-huts (Bio-Serv). Water (Innovive) and diet (Teklad Global 19% Protein Extruded Diet 2919, Irradiated) were provided ad libitum. The environment was maintained on a 12-hour light cycle at approximately 68-72° F. and 40-60% relative humidity.

Tumor Volume (TV) was calculated using the following formula: TV (mm³)=(width×width×length)/2. Tumor growth inhibition (TGI) was calculated by [(TV control_{final}−TV treated_{final})/(TV control_{final}−TV controlᵢₙᵢₜᵢₐₗ)×100]. TV was analyzed for statistical significance utilizing GraphPad Prism version 10.0.3. Repeated Measures 2-Way ANOVA with Tukey's Multiple Comparisons was utilized, and P-values were derived from data collected on Day 14 (date of Vehicle group termination) and were considered statistically significant if less than 0.05. Tumor regression was calculated by using the percent change in tumor volume from the final tumor volume measurement from Day 73 when compared to the initial tumor volume from Day 1. Tumor regression was defined as reduced TV on Day 73 compared to Day 1. Tumor regression rate was defined as the percentage of animals per group with tumor regression.

Mean tumor volume at dosing start was approximately 249 mm³, with ten mice randomized to each treatment group. Mice were dosed orally, once per day, with Vehicle, Compound A at 10 mg/kg, or dosed for 3 consecutive days followed by 4 days holiday by intraperitoneal (IP) injection of Compound B at 5 mg/kg, or combination of Compound A and Compound B. The vehicle group was a combination of Vehicle A (for Compound A, 0.5% 400 cps methylcellulose with 0.5% Tween-80 in sterile water) and Vehicle B (for irinotecan hydrochloride, Saline).

### Results

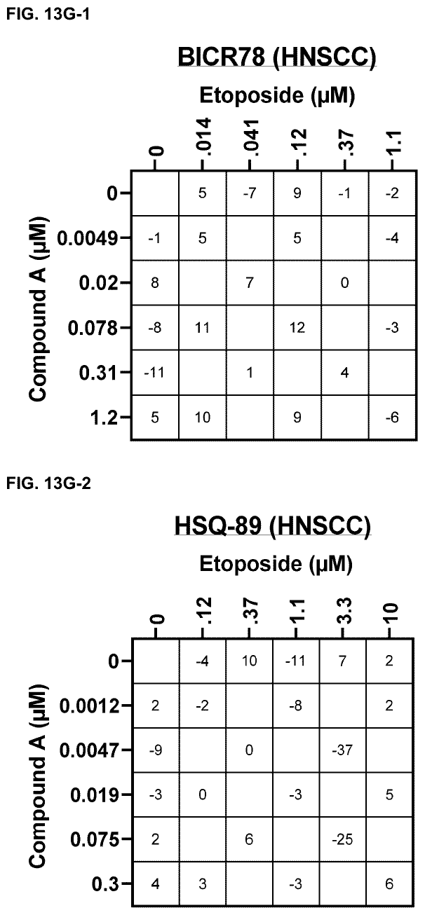

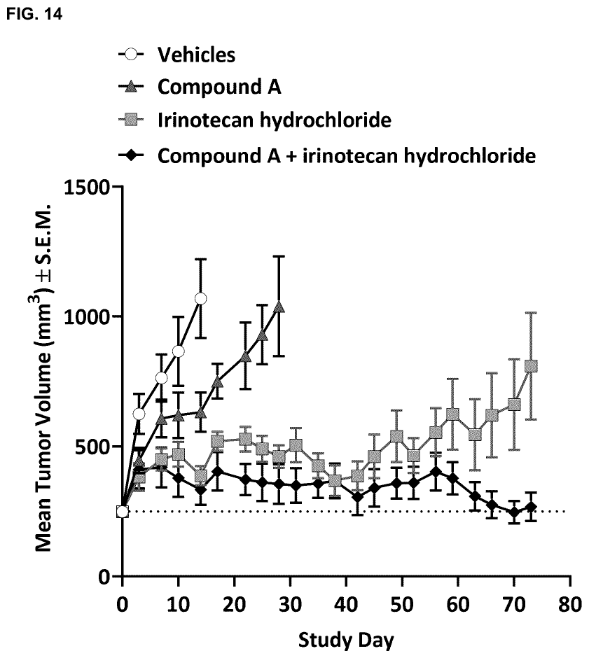

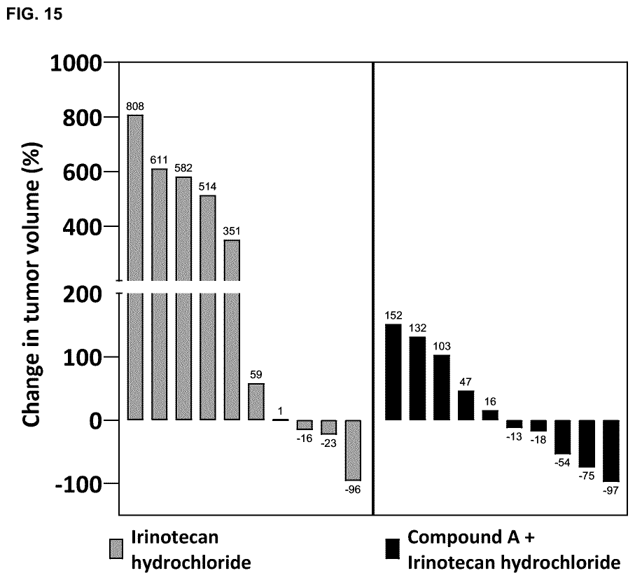

Treatment with Compound A alone resulted in 53% TGI, while irinotecan hydrochloride alone resulted in 83% TGI. The combination of Compound A and irinotecan hydrochloride resulted in 90% TGI, as shown in Table 21 and FIG. 14. The combination of Compound A and irinotecan hydrochloride increased the number of mice with tumor regressions, producing a 50% response rate on day 73, as compared to 30% produced by irinotecan hydrochloride alone, as shown in Table 21 and FIG. 15.

TABLE 21
Summary of Anti-Tumor Activity of Compound A and Compound B in RT112/84

| | Dose | | | | p value, | Tumor |
|---|---|---|---|---|---|---|
| Dose | Irinotecan | Initial | Day 14 | Day 14 | treated | Regression |
| Compound | hydrochloride | TV | TV | TGI | vs. Vehicle | Rate on |
| A (mg/kg) | (mg/kg) | (mean) | (mean) | (%) | on Day 14 | Day 73 (%) |
| 0 | 0 | 249 | 1069 | NA | NA | NA |
| 10 | 0 | 249 | 632 | 53 | ns | NA |
| 0 | 5 | 249 | 387 | 83 | 0.0065 | 30 |
| 10 | 5 | 249 | 334 | 90 | 0.0037 | 50 |

### Example 8: Anti-Tumor Activity of Compound A and Irinotecan in Gastric Mouse Xenograft Models MKN45

All mouse studies were approved by the Institutional Animal Care and Use Committee (IACUC) based on guidelines from the National Institutes of Health (NIH). Mice were maintained under pathogen-free conditions, and food and water were provided ad libitum.

The anti-tumor effect of Compound A and irinotecan hydrochloride as single-agents or in combination was assessed in the MKN45 (RCB1001) human gastric tumor cell-derived xenograft (CDX) model. MKN45 was expanded in RPMI1640 with 10% fetal bovine serum. Cells were free of mycoplasma and authenticated by STR profiling, and ten million cells in log growth phase were resuspended in Hanks Balanced Salt Solution containing 50% Matrigel and implanted subcutaneously into the flank of each recipient female Crl:NU-Foxn1nu mouse. Mice were housed in microisolator cages with corn cob bedding with additional enrichment consisting of sterile nesting material (Innovive) and Bio-huts (Bio-Serv). Water (Innovive) and diet (Teklad Global 19% Protein Extruded Diet 2919, Irradiated) were provided ad libitum. The environment was maintained on a 12-hour light cycle at approximately 68-72° F. and 40-60% relative humidity.

Tumor Volume (TV) was calculated using the following formula: TV (mm³)=(width×width×length)/2. Tumor growth inhibition (TGI) was calculated by [(TV control_{final}−TV treated_{final})/(TV control_{final}−TV controlᵢₙᵢₜᵢₐₗ)×100]. TV was analyzed for statistical significance utilizing GraphPad Prism version 10.2.2. Repeated Measures 2-Way ANOVA with Dunnet's Multiple Comparisons was utilized, and P-values were derived from data collected on the date of Vehicle group termination and were considered statistically significant if less than 0.05.

Mean tumor volume at dosing start was approximately 190 mm³, with six mice randomized to each treatment group. Mice were dosed orally, once per day, with Vehicle, Compound A at 10 mg/kg, or dosed for 3 consecutive days followed by 4 days holiday by intraperitoneal (IP) injection of Compound B at 5 mg/kg or 10 mg/kg, or combination of Compound A and Compound B. The vehicle group was a combination of Vehicle A (for Compound A, 0.5% 400 cps methylcellulose with 0.5% Tween-80 in sterile water) and Vehicle B (for irinotecan hydrochloride, Saline).

### Results

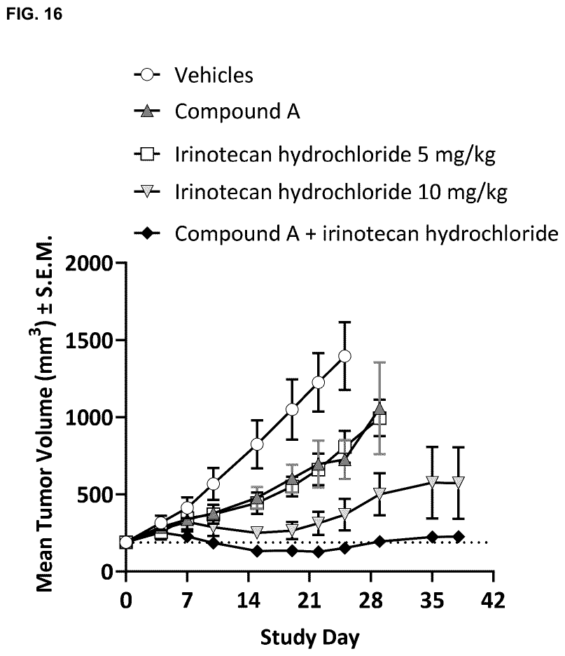

Treatment with Compound A alone resulted in 56% TGI, while irinotecan hydrochloride alone resulted in 49% TGI to 85% TGI. The combination of Compound A and irinotecan hydrochloride resulted in 103% TGI, as shown in Table 22 and FIG. 16. The combination of Compound A and irinotecan hydrochloride increased the number of mice with tumor regressions, producing an 83% tumor regression rate on day 25, as compared to 0 to 17% produced by irinotecan hydrochloride alone, as shown in Table 22.

TABLE 22
Summary of Anti-Tumor Activity of Compound A and Compound B in MKN45

| | Dose | | | | p value, | Tumor |
|---|---|---|---|---|---|---|
| Dose | Irinotecan | Initial | Day 25 | Day 25 | treated | Regression |
| Compound | hydrochloride | TV | TV | TGI | vs. Vehicle | Rate on |
| A (mg/kg) | (mg/kg) | (mean) | (mean) | (%) | on Day 25 | Day 25 (%) |
| 0 | 0 | 190 | 1396 | NA | NA | 0 |
| 10 | 0 | 191 | 726 | 56 | <0.0001 | 0 |
| 0 | 5 | 190 | 807 | 49 | <0.0001 | 0 |
| 0 | 10 | 192 | 369 | 85 | <0.0001 | 17 |
| 10 | 5 | 190 | 153 | 103 | <0.0001 | 83 |

### Example 9. Anti-Tumor Activity of Compound A and Irinotecan in Gastric Mouse Xenograft Model LMSU

All mouse studies were approved by the Institutional Animal Care and Use Committee (IACUC) based on guidelines from the National Institutes of Health (NIH). Mice were maintained under pathogen-free conditions, and food and water were provided ad libitum.

The anti-tumor effect of Compound A and irinotecan hydrochloride as single-agents or in combination was assessed in the LMSU (RCB1062) human gastric tumor cell-derived xenograft (CDX) models. LMSU was expanded in HamF10 with 10% fetal bovine serum. Cells were free of mycoplasma and authenticated by STR profiling, and ten million cells in log growth phase were resuspended in Hanks Balanced Salt Solution containing 50% Matrigel and implanted subcutaneously into the flank of each recipient female Crl:NU-Foxn1nu mouse. Mice were housed in microisolator cages with corn cob bedding with additional enrichment consisting of sterile nesting material (Innovive) and Bio-huts (Bio-Serv). Water (Innovive) and diet (Teklad Global 19% Protein Extruded Diet 2919, Irradiated) were provided ad libitum. The environment was maintained on a 12-hour light cycle at approximately 68-72° F. and 40-60% relative humidity.

Tumor Volume (TV) was calculated using the following formula: TV (mm³)=(width×width×length)/2. Tumor growth inhibition (TGI) was calculated by [(TV control_{final}−TV treated_{final})/(TV control_{final}−TV controlᵢₙᵢₜᵢₐₗ)×100]. TV was analyzed for statistical significance utilizing GraphPad Prism version 10.2.2. Repeated Measures 2-Way ANOVA with Dunnet's Multiple Comparisons was utilized, and P-values were derived from data collected on the date of Vehicle group termination and were considered statistically significant if less than 0.05.

Mean tumor volume at dosing start was approximately 185 mm³, with seven mice randomized to each treatment group. Mice were dosed orally, once per day, with Vehicle, Compound A at 10 mg/kg, or dosed for 3 consecutive days followed by 4 days holiday by intraperitoneal (IP) injection of Compound B at 5 mg/kg or 10 mg/kg, or combination of Compound A and Compound B. The vehicle group was a combination of Vehicle A (for Compound A, 0.5% 400 cps methylcellulose with 0.5% Tween-80 in sterile water) and Vehicle B (for irinotecan hydrochloride, Saline).

### Results

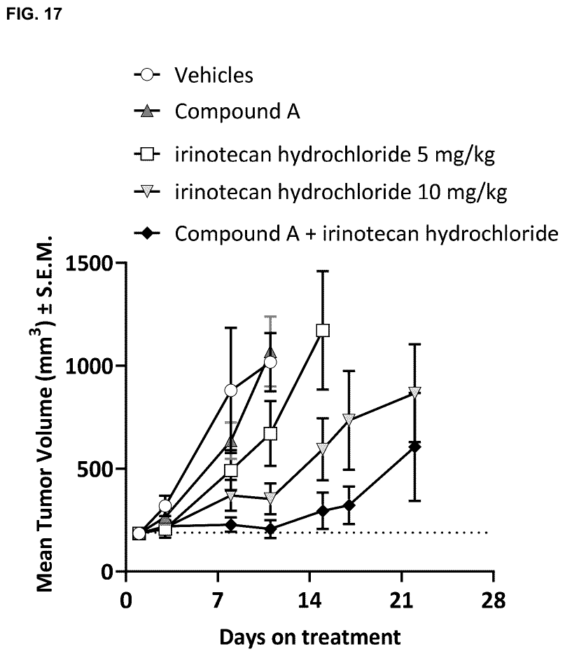

Treatment with Compound A alone resulted in no significant TGI, while irinotecan hydrochloride alone resulted in 42% TGI to 80% TGI. The combination of Compound A and irinotecan hydrochloride resulted in 97% TGI, as shown in Table 23 and FIG. 17. The combination of Compound A and irinotecan hydrochloride increased the number of mice with tumor regressions, producing a 29% tumor regression rate on day 25, as compared to 0 to 14% produced by irinotecan hydrochloride alone, as shown in Table 23.

TABLE 23
Summary of Anti-Tumor Activity of Compound A and Compound B in LMSU

| | Dose | | | | p value, | Tumor |
|---|---|---|---|---|---|---|
| Dose | Irinotecan | Initial | Day 11 | Day 11 | treated | Regression |
| Compound | hydrochloride | TV | TV | TGI | vs. Vehicle | Rate on |
| A (mg/kg) | (mg/kg) | (mean) | (mean) | (%) | on Day 11 | Day 11 (%) |
| 0 | 0 | 185 | 1018 | NA | NA | 0 |
| 10 | 0 | 186 | 1069 | −6 | ns | 0 |
| 0 | 5 | 185 | 671 | 42 | 0.0016 | 0 |
| 0 | 10 | 185 | 353 | 80 | <0.0001 | 14 |
| 10 | 10 | 186 | 206 | 97 | <0.0001 | 29 |

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

Other embodiments are within the following claims.

1. A method of treating cancer in a subject in need thereof comprising administering to the subject a therapeutically effective amount of a MAT2A inhibitor and administering to the subject a therapeutically effective amount of a topoisomerase inhibitor, wherein the MAT2A inhibitor is a compound of Formula (I) or a pharmaceutically acceptable salt thereof: 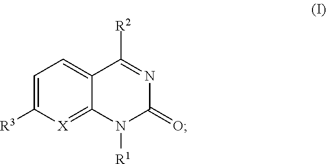
wherein
X is N or CH;
R³ is C₁₋₆ haloalkyl, halo, or C₃₋₆ cycloalkyl;
R² is —NR⁴R⁵;
R⁴ is hydrogen or C₁₋₆ alkyl;
R⁵ is hydrogen, C₁₋₆ alkyl, or C₃₋₆ cycloalkyl; and
R¹ is phenyl substituted with 0-2 halo.

2. The method according to claim 1, wherein the topoisomerase inhibitor is a Type I topoisomerase inhibitor.

3. The method according to claim 2, wherein the Type I topoisomerase inhibitor is selected from the group consisting of 10-hydroxycamptothecin, irinotecan, hexylresorcinol, exatecan, deruxtecan, belotecan, and topotecan, or a pharmaceutically acceptable salt thereof.

4. The method according to claim 3, wherein the Type I topoisomerase inhibitor is irinotecan or a pharmaceutically acceptable salt thereof.

5. The method according to claim 1, wherein the topoisomerase inhibitor is a Type II topoisomerase inhibitor.

6. The method according to claim 5, wherein the Type II topoisomerase inhibitor is selected from the group consisting of daunorubicin, doxorubicin, and etoposide, or a pharmaceutically acceptable salt thereof.

7. The method according to any one of claims 1 to 6, wherein the MAT2A inhibitor is administered simultaneously or sequentially with the topoisomerase inhibitor.

8. A method of treating cancer in a subject in need thereof comprising administering to the subject a therapeutically effective amount of a MAT2A inhibitor and administering to the subject a therapeutically effective amount of a splicing inhibitor sulfonamide (SPLAM), wherein the MAT2A inhibitor is a compound of Formula (I) or a pharmaceutically acceptable salt thereof: 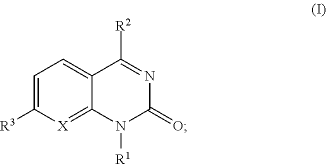
wherein
X is N or CH;
R³ is C₁₋₆ haloalkyl, halo, or C₃₋₆ cycloalkyl;
R² is —NR⁴R⁵;
R⁴ is hydrogen or C₁₋₆ alkyl;
R⁵ is hydrogen, C₁₋₆ alkyl, or C₃₋₆ cycloalkyl; and
R¹ is phenyl substituted with 0-2 halo.

9. The method according to claim 8, wherein the SPLAM is indisulam, or a pharmaceutically acceptable salt thereof.

10. The method according to claim 8, wherein the SPLAM is selected from the group consisting of E7820, chloroquinoxaline sulfonamide, and tasisulam, or a pharmaceutically acceptable salt thereof.

11. The method according to any one of claims 8 to 10, wherein the MAT2A inhibitor is administered simultaneously or sequentially with the SPLAM.

12. A method of treating cancer in a subject in need thereof comprising administering to the subject a therapeutically effective amount of a MAT2A inhibitor and administering to the subject a therapeutically effective amount of a KIF18 inhibitor, wherein the MAT2A inhibitor is a compound of Formula (I) or a pharmaceutically acceptable salt thereof: 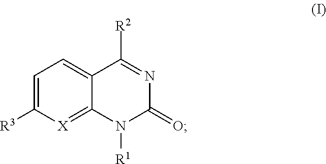
wherein
X is N or CH;
R³ is C₁₋₆ haloalkyl, halo, or C₃₋₆ cycloalkyl;
R² is —NR⁴R⁵;
R⁴ is hydrogen or C₁₋₆ alkyl;
R⁵ is hydrogen, C₁₋₆ alkyl, or C₃₋₆ cycloalkyl; and
R¹ is phenyl substituted with 0-2 halo.

13. The method according to claim 12, wherein the KIF18 inhibitor is a KIF18A inhibitor.

14. The method according to claim 13, wherein the KIF18A inhibitor is sovilnesib, or a pharmaceutically acceptable salt thereof.

15. The method of any one of claims 12 to 14, wherein the MAT2A inhibitor is administered simultaneously or sequentially with the KIF18 inhibitor.

16. The method according to any one of claims 1 to 15, wherein X is N.

17. The method according to any one of claims 1 to 15, wherein X is CH.

18. The method according to any one of claims 1 to 17, wherein R⁴ is hydrogen and R⁵ is hydrogen or C₁₋₃ alkyl.

19. The method according to any one of claims 1 to 18, wherein R⁵ is hydrogen or methyl.

20. The method according to any one of claims 1 to 19, wherein R¹ is phenyl substituted with chloro.

21. The method according to any one of claims 1 to 20, wherein R³ is C₁₋₃ haloalkyl or halo.

22. The method according to any one of claims 1 to 21, wherein R³ is trifluoromethyl or chloro.

23. The method according to any one of claims 1 to 21, wherein R³ is cyclopropyl.

24. The method according to any one of claims 1 to 23, wherein the MAT2A inhibitor is selected from the group consisting of a compound from Table 1, or a pharmaceutically acceptable salt thereof.

25. The method according to any one of claims 1 to 24, wherein the MAT2A inhibitor is Compound A: 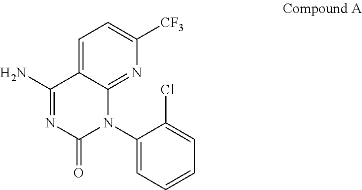 [SMILES: Nc1nc(=O)n(-c2ccccc2Cl)c2nc(C(F)(F)F)ccc12]
or a pharmaceutically acceptable salt thereof.

26. The method according to any one of claims 1 to 24, wherein the MAT2A inhibitor is Compound A1: 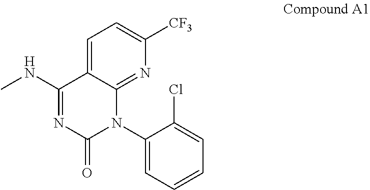 [SMILES: CNc1nc(=O)n(-c2ccccc2Cl)c2nc(C(F)(F)F)ccc12]
or a pharmaceutically acceptable salt thereof.

27. The method according to any one of claims 1 to 26, wherein the cancer is selected from the group consisting of leukemia, glioma, lung cancer, esophageal cancer, MTAP-deficient pancreatic ductal adenocarcinoma (PDAC), melanoma, pancreatic, non-small cell lung cancer, bladder cancer, astrocytoma, osteosarcoma, head and neck cancer, myxoid chondrosarcoma, ovarian cancer, endometrial cancer, breast cancer, anal cancer, stomach cancer, colon cancer, colorectal cancer, soft tissue sarcoma, non-Hodgkin lymphoma, gastric cancer, esophagogastric cancer, malignant peripheral nerve sheath tumor, mesothelioma, salivary gland tumors, urothelial cancers, gastrointestinal cancer, and sarcomas.

28. The method according to any one of claims 1 to 27, wherein the cancer is a solid tumor or a hematological cancer.

29. The method according to any one of claims 1 to 28, wherein the cancer is a solid, malignant tumor.

30. The method according to any one of claims 1 to 29, wherein the cancer is characterized by a reduction or absence of MTAP gene expression, an absence of MTAP gene, a reduced function of MTAP protein, a reduced level or absence of MTAP protein, a MTA accumulation, or a combination thereof.

31. Use of a MAT2A inhibitor in the manufacture of a medicament for treating cancer, wherein the MAT2A inhibitor is used in combination with a topoisomerase inhibitor.

32. Use of a MAT2A inhibitor in the manufacture of a medicament for treating cancer, wherein the MAT2A inhibitor is used in combination with a SPLAM.

33. Use of a MAT2A inhibitor in the manufacture of a medicament for treating cancer, wherein the MAT2A inhibitor is used in combination with a KIF18 inhibitor.

34. The use of any one of claims 31 to 33, wherein the MAT2A inhibitor is a compound of Formula (I), or a pharmaceutically acceptable salt thereof.

35. The use of claim 34, wherein the MAT2A inhibitor is Compound A or a pharmaceutically acceptable salt thereof, or Compound A1 or a pharmaceutically acceptable salt thereof.

36. A method of inhibiting tumor growth or slowing the rate of tumor growth in a subject having a MTAP-deficient cancer, the method comprising administration of a therapeutically effective amount of a MAT2A inhibitor and a therapeutically effective amount of a topoisomerase inhibitor, a SPLAM, or a KIF18 inhibitor.

37. The method according to claim 36, wherein the tumor growth is measured by a change in a tumor volume from a first time point to a second time point.

38. The method according to claim 37, wherein the tumor volume at the second time point has no increase when compared to the first time point.

39. The method according to claim 38, wherein the tumor volume decreases from the first time point to the second time point.

40. A method of treating cancer in a subject in need thereof, the method comprising administering to the subject a therapeutically effective amount of a MAT2A inhibitor and a therapeutically effective amount of a topoisomerase inhibitor, a SPLAM, or a KIF18 inhibitor,
wherein the subject is not previously treated with a MAT2A inhibitor.

41. The method according to claim 40, wherein the treatment decreases the rate of tumor growth when compared to a treatment with the MAT2A inhibitor alone for a similar time period.
